(19) United States
(12) Patent Application Publication
(10) Pub. No.: US 2023/0099759 A1
(43) Pub. Date: Mar. 30, 2023

(54) POLYCYCLIC COMPOUND, MATERIAL FOR AN ORGANIC ELECTROLUMINESCENCE DEVICE AND AN ORGANIC ELECTROLUMINESCENCE DEVICE COMPRISING THE POLYCYCLIC COMPOUND

(71) Applicant: IDEMITSU KOSAN CO.,LTD., Chiyoda-ku (JP)

(72) Inventors: Michelle GROARKE, Binningen (CH); Heinz WOLLEB, Fehren (CH); Natalia CHEBOTAREVA, Hagenthal le Bas (FR)

(73) Assignee: IDEMITSU KOSAN CO.,LTD., Chiyoda-ku (JP)

(21) Appl. No.: 17/786,789

(22) PCT Filed: Dec. 18, 2020
(86) PCT No.: PCT/JP2020/049306

§ 371 Date: Jun. 17, 2022

(30) Foreign Application Priority Data
Dec. 23, 2019 (EP) 19219231.8

(51) Int. Cl. H01L 51/00; C07D 495/04; C07D 491/04

(52) U.S. Cl. CPC H01L 51/0067; C07D 495/04; C07D 491/04; H01L 51/0071; H01L 51/5076

(57) ABSTRACT

An organic electroluminescence device includes an anode, a cathode, and one or more organic thin film layers including an emitting layer disposed between the cathode and the anode. Also, the organic thin film layer includes an electron-transporting zone provided between the emitting layer and the cathode. The organic thin film layer contains at least one compound of the polycyclic compound (Ia) or (Ib):

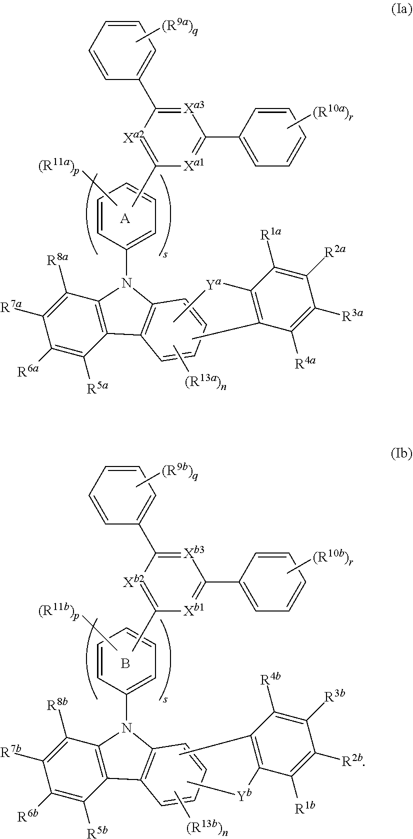

### TECHNICAL FIELD

The present invention relates to specific compounds, a material for an organic electroluminescence device comprising said specific compound, an organic electroluminescence device comprising said specific compound, an electronic equipment comprising said organic electroluminescence device and the use of said compounds in an organic electroluminescence device.

### BACKGROUND ART

When a voltage is applied to an organic electroluminescence device (hereinafter may be referred to as an organic EL device), holes are injected to an emitting layer from an anode and electrons are injected to an emitting layer from a cathode. In the emitting layer, injected holes and electrons are re-combined and excitons are formed.

An organic EL device comprises an emitting layer between the anode and the cathode. Further, there may be a case where it has a stacked layer structure comprising an organic layer such as a hole-injecting layer, a hole-transporting layer, an electron-blocking layer, an electron-injecting layer, an electron-transporting layer, a hole-blocking layer etc.

WO2016/018076 A1 relates to an electron buffering material comprising a compound represented by the following formula 1, and an organic electroluminescent device comprising a first electrode; a second electrode facing the first electrode; a light-emitting layer between the first electrode and the second electrode; and an electron transport zone and an electron buffering layer between the light-emitting layer and the second electrode; wherein the electron buffering layer comprises a compound represented by the following formula 1:

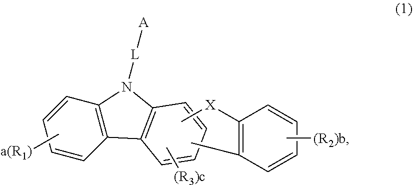

wherein L represents a single bond, a substituted or unsubstituted (C6-C30)arylene, or a substituted or unsubstituted 5- to 30-membered heteroarylene; and A represents a substituted or unsubstituted 5- to 30-membered heteroaryl.

Suitable compounds exemplified in WO2016/018076 A1 are for example the following compounds:

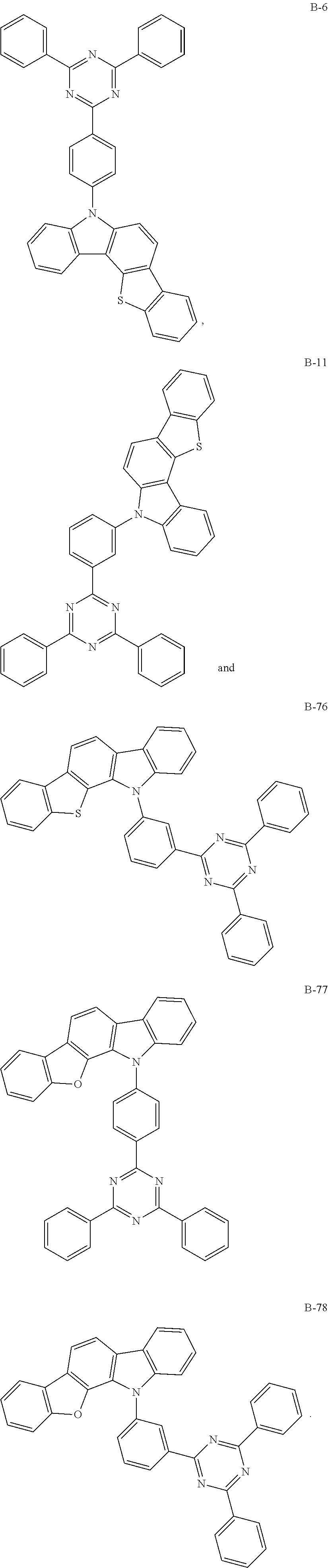

KR 10-2018-0071609 A relates—in one embodiment—to a composition for an organic layer of an organic light emitting device, comprising a heterocyclic compound represented by chemical formula 1 and a compound represented by chemical formula 2 at the same time.

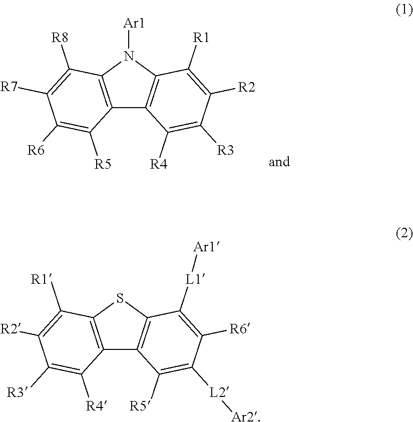

Numerous examples for compounds of formula (1) and (2) are disclosed in KR 2018-0071609 A, for example the following compounds (of formula (2)):

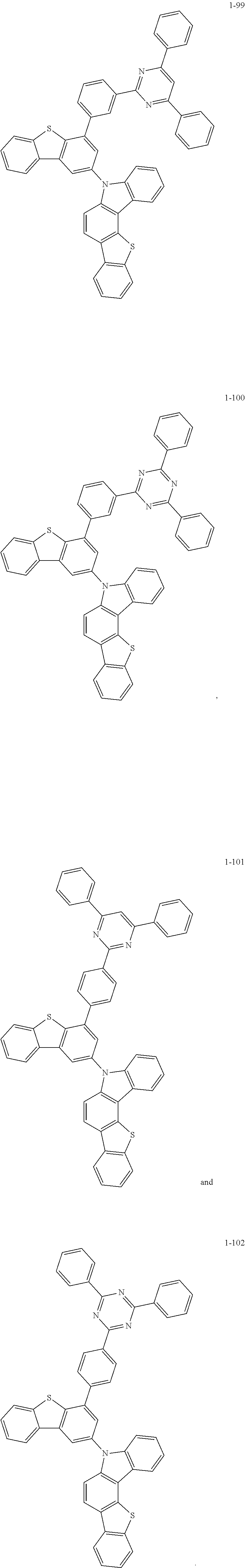 [SMILES: c1ccc(-c2cc(-c3ccccc3)nc(-c3cccc(-c4cc(-n5c6ccccc6c6c7sc8ccccc8c7ccc65)cc5c4sc4ccccc45)c3)n2)cc1 ; c1ccc(-c2nc(-c3ccccc3)nc(-c3cccc(-c4cc(-n5c6ccccc6c6c7sc8ccccc8c7ccc65)cc5c4sc4ccccc45)c3)n2)cc1 ; c1ccc(-c2cc(-c3ccccc3)nc(-c3ccc(-c4cc(-n5c6ccccc6c6c7sc8ccccc8c7ccc65)cc5c4sc4ccccc45)cc3)n2)cc1 ; c1ccc(-c2nc(-c3ccccc3)nc(-c3ccc(-c4cc(-n5c6ccccc6c6c7sc8ccccc8c7ccc65)cc5c4sc4ccccc45)cc3)n2)cc1]

WO 2019/054833 A1 relates to a heterocyclic compound of formula (1) and an organic light emitting device including the same:

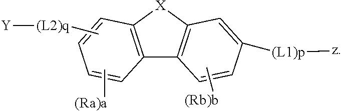

Among numerous exemplified compounds, the following compounds are disclosed:

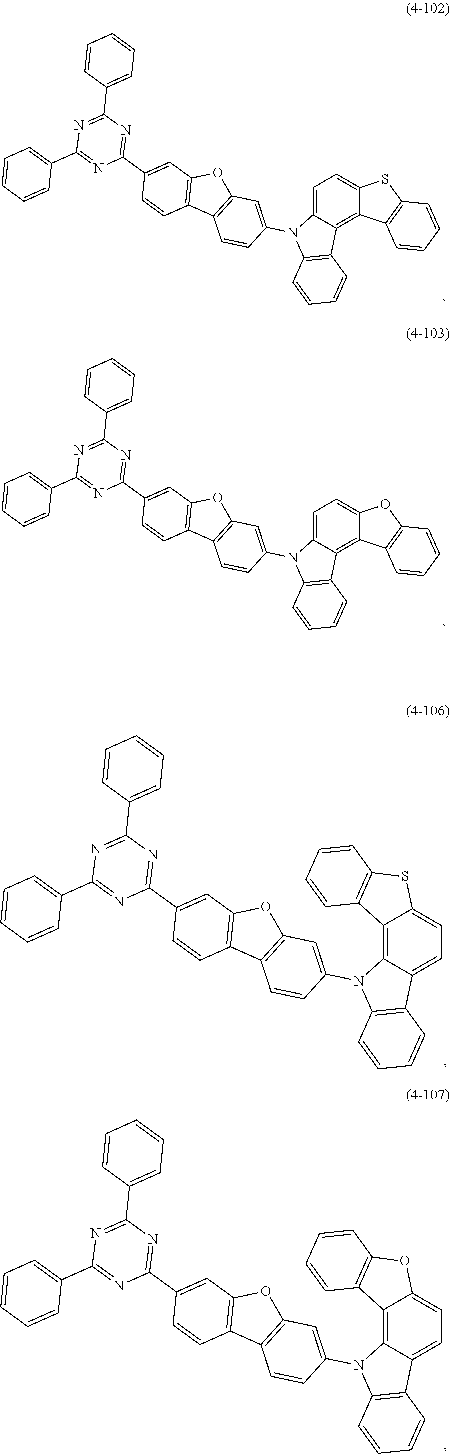 [SMILES: c1ccc(-c2nc(-c3ccccc3)nc(-c3ccc4c(c3)oc3cc(-n5c6ccccc6c6c7c(ccc65)sc5ccccc57)ccc34)n2)cc1 ; c1ccc(-c2nc(-c3ccccc3)nc(-c3ccc4c(c3)oc3cc(-n5c6ccccc6c6c7c(ccc65)oc5ccccc57)ccc34)n2)cc1 ; c1ccc(-c2nc(-c3ccccc3)nc(-c3ccc4c(c3)oc3cc(-n5c6ccccc6c6ccc7sc8ccccc8c7c65)ccc34)n2)cc1 ; c1ccc(-c2nc(-c3ccccc3)nc(-c3ccc4c(c3)oc3cc(-n5c6ccccc6c6ccc7oc8ccccc8c7c65)ccc34)n2)cc1]

KR 10-2014-0006708 A provides an organic electroluminescent compound represented by the chemical formula (1) and an organic electroluminescent device including the same. The organic electroluminescent compound is a green phosphorescent host material, can be applied to the organic electroluminescent device, and can improve the luminous efficiency of the organic electroluminescent device and the lifetime of the device.

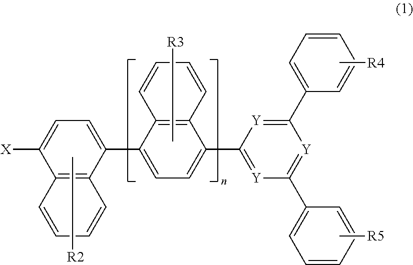

Under numerous exemplified compounds, for example the following compound is disclosed:

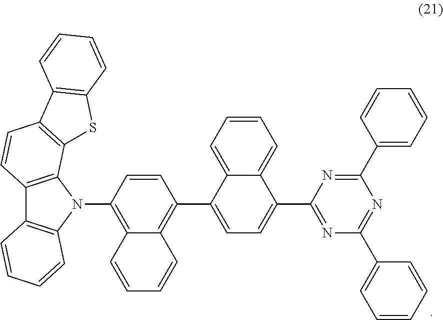 [SMILES: c1ccc(-c2nc(-c3ccccc3)nc(-c3ccc(-c4ccc(-n5c6ccccc6c6ccc7c8ccccc8sc7c65)c5ccccc45)c4ccccc34)n2)cc1]

WO 2019/027263 A1 provides a heterocyclic compound of formula (1) and an organic light emitting device including the same.

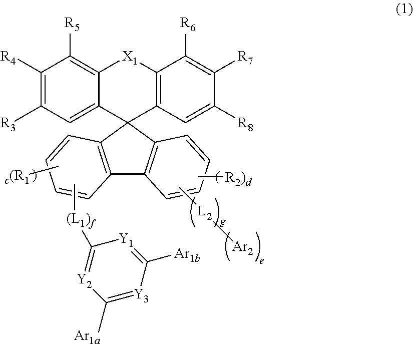

Under numerous exemplified compounds, for example the following compounds are disclosed:

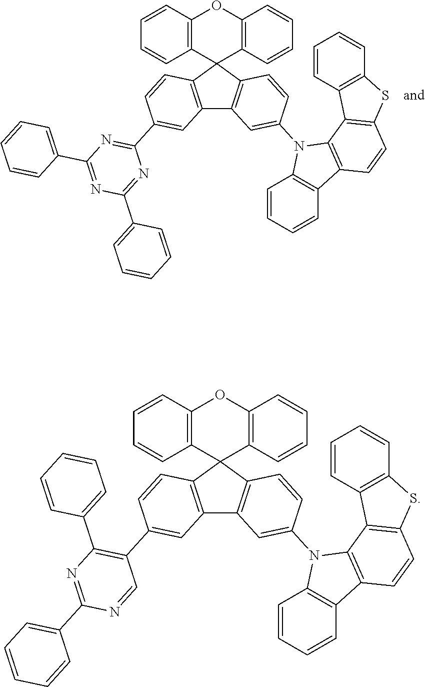 [SMILES: c1ccc(-c2nc(-c3ccccc3)nc(-c3ccc4c(c3)-c3cc(-n5c6ccccc6c6ccc7sc8ccccc8c7c65)ccc3C43c4ccccc4Oc4ccccc43)n2)cc1 ; c1ccc(-c2ncc(-c3ccc4c(c3)-c3cc(-n5c6ccccc6c6ccc7sc8ccccc8c7c65)ccc3C43c4ccccc4Oc4ccccc43)c(-c3ccccc3)n2)cc1]

### CITATION LIST

### Patent Literature

WO 2016/018076 A1
KR 10-2018-0071609 A
WO 2019/054833 A1
KR 10-2014-0006708 A
WO 2019/027263 A1

### SUMMARY OF INVENTION

### Technical Problem

The specific structure and substitution pattern of polycyclic compounds have a significant impact on the performance of the polycyclic compounds in organic electronic devices.

Therefore, notwithstanding the developments described above, there remains a need for organic electroluminescence devices comprising new materials, especially charge-transporting materials, e.g. electron-transporting materials, charge-blocking materials, e.g. hole-blocking materials and/or dopant materials, to provide improved performance of electroluminescence devices.

Accordingly, it is an object of the present invention, with respect to the aforementioned related art, to provide further materials suitable for use in organic electroluminescence devices and further applications in organic electronics. More particularly, it should be possible to provide charge-transporting materials, e.g. electron-transporting materials, and/or charge-blocking materials, e.g. hole-blocking materials, and/or dopant materials for use in organic electroluminescence devices. The materials should be suitable especially for organic electroluminescence devices which comprise at least one emitter, which is a phosphorescence emitter and/or a fluorescence emitter.

Furthermore, the materials should be suitable for providing organic electroluminescence devices which ensure good performance of the organic electroluminescence devices, especially a high external quantum efficiency (EQE), long lifetime and/or low driving voltage.

### Solution to Problem

Said object is solved by a polycyclic compound represented by formula (Ia) or (Ib):

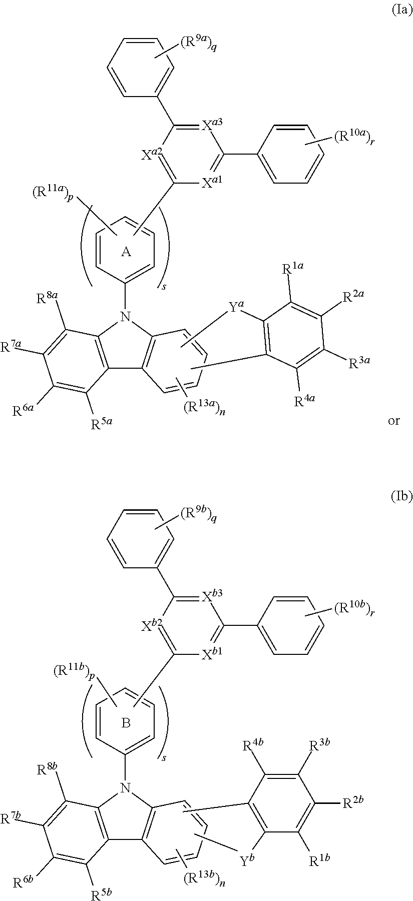

wherein

R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN;

R^{9a}, R^{10a}, R^{9b} and R^{10b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN, or

two adjacent groups R^{9a}, and/or two adjacent groups R^{10a}, or two adjacent groups R^{9b}, and/or two adjacent groups R^{10b} can form together a substituted or unsubstituted carbocyclic or heterocyclic ring;

R^{11a} and R^{11b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN, or

two groups R^{11a} on adjacent rings A or two groups R^{11b} on adjacent rings B can form together an unsubstituted carbocyclic ring or a carbocyclic ring substituted by one or more heteroatom-free substituents, and/or

two adjacent groups R^{11a} on one of the rings A or two adjacent groups R^{11b} on one of the rings B can form together a substituted or unsubstituted carbocyclic ring;

X^{a1}, X^{a2}, X^{a3}, X^{b1}, X^{b2} and X^{b3} each independently represents N or CR¹², wherein at least two of X^{a1}, X^{a2} and X^{a3} and at least two of X^{b1}, X^{b2} and X^{b3} are N;

R¹² represents in each case independently hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN;

Y^{a} and Y^{b} each independently represents S or O;

n is 1 or 2;

q and r are each independently 1, 2, 3, 4 or 5;

p is 1, 2, 3 or 4; and

s is 2, 3 or 4.

The specific polycyclic compounds of the present invention according to formulae (Ia) and (Ib) may be used as a material, especially host, charge-transporting or charge-blocking material, that is highly suitable in organic electroluminescence devices. Moreover, thermally stable compounds are provided, especially resulting in organic electroluminescence devices having high external quantum efficiencies (EQE), long lifetime and/or low driving voltages.

The compounds of the present invention may also be used in further organic electronic devices than organic electroluminescence devices such as electrophotographic photoreceptors, photoelectric converters, organic solar cells (organic photovoltaics), switching elements, such as organic transistors, for example, organic FETs and organic TFTs, organic light emitting field effect transistors (OLEFETs), image sensors and dye lasers.

Accordingly, a further subject of the present invention is directed to an organic electronic device, comprising a compound according to the present invention. The organic electronic device is preferably an organic electroluminescence device (EL device). The term organic EL device (organic electroluminescence device) is used interchangeably with the term organic light-emitting diode (OLED) in the present application.

The compounds of formulae (Ia) and (Ib) can in principal be used in any layer of an EL device, but are preferably used as charge-transporting, especially electron-transporting, charge-blocking, especially hole-blocking, material. Particularly, the compounds of formulae (Ia) and (Ib) are used as electron-transporting material and/or hole-blocking material for phosphorescence or fluorescence emitters. More preferably, the compounds of formulae (Ia) and (Ib) are used as electron-transporting material for phosphorescence or fluorescence emitters.

Hence, a further subject of the present invention is directed to a material for an organic electroluminescence device comprising at least one compound of formula (Ia) or (Ib) according to the present invention.

A further subject of the present invention is directed to an organic electroluminescence device which comprises an organic thin film layer between a cathode and an anode, wherein the organic thin film layer comprises one or more layers and comprises a light emitting layer, and at least one layer of the organic thin film layer comprises at least one compound of formula (Ia) or (Ib) according to the present invention.

A further subject of the present invention is directed to an electronic equipment comprising the organic electroluminescence device according the present invention.

A further subject of the present invention is directed to the use of a compound of formula (Ia) or (Ib) according to the present invention in an organic electroluminescence device.

A further subject of the present invention is directed to an emitting layer, comprising a compound of formula (Ia) or (Ib) according to the present invention. In said embodiment the compound of formula (Ia) or (Ib) is preferably used in the electron-transporting zone. In the meaning of the present invention, the electron-transporting zone includes at least an electron-transporting layer and preferably also an electron-injection layer and/or a hole-blocking layer.

A further subject of the present invention is directed to an electron-transporting layer comprising a compound of formula (Ia) or (Ib) according to the present invention. Preferably, the electron-transporting layer is provided between the cathode and the light emitting layer of an EL device such as an OLED.

A further subject of the present invention is directed to a hole-blocking layer comprising a compound of formula (Ia) or (Ib) according to the present invention. Preferably, the hole-blocking layer is provided between the electron-transporting layer and the light emitting layer of an EL device such as an OLED.

### Advantageous Effects of Invention

The compounds of the invention are suitable for providing organic electroluminescence devices which ensure good performance of the organic electroluminescence devices, especially a high external quantum efficiency (EQE), long lifetime and/or low driving voltage.

### BRIEF DESCRIPTION OF DRAWINGS

FIG. 1 shows a schematic configuration of one example of the organic EL device of the invention.

### DESCRIPTION OF EMBODIMENTS

The terms carbocyclic group having 5 to 18 ring carbon atoms, heterocyclic group having 5 to 24 ring atoms, a heterocyclic group having 5 to 18 ring atoms, heteroaryl group having 5 to 18 ring atoms, an aryl group having 6 to 18 ring carbon atoms, alkyl group having 1 to 25 carbon atoms, preferably 1 to 8 carbon atoms, a cycloalkyl group having 3 to 6 ring carbon atoms, alkoxy group having 1 to 25, preferably 1 to 8 carbon atoms, are known in the art and generally have the following meaning, if said groups are not further specified in specific embodiments mentioned below:

The carbocyclic group having 5 to 18 ring carbon atoms is preferably an aromatic hydrocarbon group having a ring structure formed of 6 to 18 carbon atoms, a cycloalkenyl group having 5 to 18 ring carbon atoms or a cycloalkyl group having 5 to 18 ring carbon atoms.

The aryl group having 6 to 18 ring carbon atoms may be a non-condensed aryl group or a condensed aryl group.

Specific examples thereof include phenyl group, naphthyl group, phenanthryl group, biphenyl group, terphenyl group, quaterphenyl group, fluoranthenyl group, triphenylenyl group, phenanthrenyl group, fluorenyl group, anthracenyl, chrysenyl, spirofluorenyl group, 9,9-diphenylfluorenyl group, 9,9′-spirobi[9H-fluorene]-2-yl group, 9,9-dimethylfluorenyl group, benzo[c]phenanthrenyl group, benzo[a]triphenylenyl group, naphtho[1,2-c]phenanthrenyl group, naphtho[1,2-a]triphenylenyl group, dibenzo[a,c]triphenylenyl group, benzo[a]fluoranthenyl group, benzo[j]fluoranthenyl group, benzo[k]fluoranthenyl group and benzo[b]fluoranthenyl group, with phenyl group, naphthyl group, biphenyl group, terphenyl group, phenanthryl group, triphenylenyl group, fluorenyl group, spirobifluorenyl group, and fluoranthenyl group being preferred, and phenyl group, 1-naphthyl group, 2-naphthyl group, biphenyl-2-yl group, biphenyl-3-yl group, biphenyl-4-yl group, phenanthrene-9-yl group, phenanthrene-3-yl group, phenanthrene-2-yl group, triphenylene-2-yl group, 9,9-dimethylfluorene-2-yl group, fluoranthene-3-yl group, fluoranthene-2-yl group, fluoranthene-8-yl group being more preferred.

Examples of the cycloalkyl group having 5 to 18 ring carbon atoms, preferably 5 to 12 ring carbon atoms include cyclopentyl group, cyclohexyl group, cyclooctyl group, and adamantyl group, with cyclopentyl group, and cyclohexyl group being preferred. Preferred are cycloalkyl groups having 5 or 6 ring carbon atoms. Suitable examples for cycloalkyl groups having 5 or 6 ring carbon atoms are mentioned before.

Examples of the cycloalkyl group having 3 to 6 ring carbon atoms include cyclopropyl group, cyclobutyl group, cyclopentyl group, and cyclohexyl group.

The heterocyclic group having a ring structure formed of 5 to 18 ring atoms (heterocyclic group having 5 to 18 ring atoms) may be a non-condensed heterocyclic group or a condensed heterocyclic group. Preferably, the heterocyclic group having 5 to 18 ring atoms is a heteroaryl group having 5 to 18 ring atoms.

Specific examples thereof include the residues of pyrrole ring, isoindole ring, benzofuran ring, isobenzofuran ring, benzothiophene, dibenzothiophene ring, isoquinoline ring, quinoxaline ring, quinazoline, phenanthridine ring, phenanthroline ring, pyridine ring, pyrazine ring, pyrimidine ring, pyridazine ring, indole ring, quinoline ring, acridine ring, carbazole ring, furan ring, thiophene ring, benzoxazole ring, benzothiazole ring, benzimidazole ring, dibenzofuran ring, triazine ring, oxazole ring, oxadiazole ring, thiazole ring, thiadiazole ring, triazole ring, and imidazole ring, with the residues of dibenzofuran ring, carbazole ring, and dibenzothiophene ring being preferred, and the residues of dibenzofuran-1-yl group, dibenzofuran-3-yl group, dibenzofuran-2-yl group, dibenzofuran-4-yl group, 9-phenylcarbazole-3-yl group, 9-phenylcarbazole-2-yl group, 9-phenylcarbazole-4-yl group, dibenzothiophene-2-yl group, and dibenzothiophene-4-yl, dibenzothiophene-1-yl group, and dibenzothiophene-3-yl group being more preferred.

Examples of the alkyl group having 1 to 25 carbon atoms, preferably 1 to 8 carbon atoms, are methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, and 1-methylpentyl group, with methyl group, ethyl group, n-propyl group, isopropyl group, n-butyl group, s-butyl group, isobutyl group, and t-butyl group being preferred.

Examples of an alkoxy group having 1 to 25, preferably 1 to 8 carbon atoms, are alkoxy groups —OR^{a}, wherein R^{a} is an alkyl group having 1 to 25, preferably 1 to 8 carbon atoms, i.e. methyl group, ethyl group, n-propyl group, isopropyl group, n-butyl group, s-butyl group, isobutyl group, t-butyl group, n-pentyl group, n-hexyl group, n-heptyl group, and n-octyl group.

Examples of the optional substituent(s) indicated by “substituted or unsubstituted” and “may be substituted” referred to above or hereinafter include a halogen atom (fluorine, chlorine, bromine, iodine), a cyano group, an alkyl group having 1 to 25, preferably 1 to 8 carbon atoms, a cycloalkyl group having 5 to 18, preferably 5 to 12 ring carbon atoms, an alkoxy group having 1 to 25, preferably 1 to 8 carbon atoms, a haloalkyl group having 1 to 25, preferably 1 to 5 carbon atoms, a haloalkoxyl group having 1 to 25, preferably 1 to 5 carbon atoms, an alkylamino group having 1 to 25 carbon atoms, preferably 1 to 5 carbon atoms, a carboxyalkyl group having 1 to 25 carbon atoms, preferably 1 to 5 carbon atoms, a carboxamidalkyl group having 1 to 25 carbon atoms, preferably 1 to 5 carbon atoms, a silyl group, an aromatic hydrocarbon group having 6 to 24 ring carbon atoms, preferably 6 to 18 ring carbon atoms, an aryloxy group having 6 to 24, preferably 6 to 18 ring carbon atoms, an aralkyl group having 7 to 24, preferably 7 to 20 carbon atoms, an alkylthio group having 1 to 25, preferably 1 to 5 carbon atoms, an arylthio group having 6 to 24, preferably 6 to 18 ring carbon atoms, an arylamino group having 6 to 30 ring carbon atoms, preferably 6 to 18 ring carbon atoms, a carboxyaryl group having 6 to 24 carbon atoms, preferably 6 to 18 carbon atoms, a carboxamidaryl group having 6 to 24 carbon atoms, preferably 6 to 18 carbon atoms, and an heterocyclic group having 5 to 24 ring atoms, preferably 5 to 18 ring atoms.

The optional substituent is preferably a fluorine atom, a cyano group, an alkyl group having 1 to 25 carbon atoms, an aromatic hydrocarbon group having 6 to 24 ring carbon atoms, preferably 6 to 18 ring carbon atoms, and an heterocyclic group having 5 to 24 ring atoms, preferably 5 to 18 ring atoms; more preferably a cyano group, a phenyl group, a naphthyl group, a biphenyl group, a terphenyl group, a phenanthryl group, a triphenylenyl group, a fluorenyl group, a spirobifluorenyl group, a fluoranthenyl group, a residue based on a dibenzofuran ring, a residue based on a carbazole ring, and a residue based on a dibenzothiophene ring, a methyl group, an ethyl group, an n-propyl group, an isopropyl group, an n-butyl group, an s-butyl group, an isobutyl group, a t-butyl group, a cyclopentyl group, and a cyclohexyl group.

The optional substituent mentioned above may be further substituted by one or more of the optional substituents mentioned above.

The number of the optional substituents depends on the group which is substituted by said substituent(s). Preferred are 1, 2, 3 or 4 optional substituents, more preferred are 1, 2 or 3 optional substituents, most preferred are 1 or 2 optional substituents. In a further preferred embodiment, the groups mentioned above are unsubstituted.

The “carbon number of a to b” in the expression of “substituted or unsubstituted X group having a to b carbon atoms” is the carbon number of the unsubstituted X group and does not include the carbon atom(s) of an optional substituent.

The hydrogen atom referred to herein includes isotopes different from neutron numbers, i.e., light hydrogen (protium), heavy hydrogen (deuterium) and tritium.

The term “unsubstituted” referred to by “unsubstituted or substituted” means that a hydrogen atom is not substituted by one the groups mentioned above.

R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b}

R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN.

Preferably, R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted carbocyclic group having 5 to 18 ring carbon atoms, a substituted or unsubstituted heterocyclic group having 5 to 18 ring atoms, or CN.

More preferably, R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted phenyl group, CN, a substituted or unsubstituted biphenyl group, a substituted or unsubstituted terphenyl group, a substituted or unsubstituted triphenylenyl group, a substituted or unsubstituted naphthyl group, a substituted or unsubstituted phenanthrenyl group, a substituted or unsubstituted dibenzofuranyl group, or a substituted or unsubstituted dibenzothiophenyl group.

Most preferably, R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each represents hydrogen, a substituted or unsubstituted phenyl group, a substituted or unsubstituted biphenyl group, or CN, further most preferably hydrogen or a substituted or unsubstituted phenyl group.

In a preferred embodiment of the present invention, 0, 1, 2 or 3 of the residues R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} are not hydrogen, more preferably 0, 1 or 2 of the residues R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} are not hydrogen, further more preferably 0 or 1 of the residues R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} are not hydrogen.

Most preferably, R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} are hydrogen.

R^{9a}, R^{10a}, R^{9b} and R^{10b}

R^{9a}, R^{10a}, R^{9b} and R^{10b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN, or two adjacent groups R^{9a}, and/or two adjacent groups R^{10a}, or two adjacent groups R^{9b}, and/or two adjacent groups R^{10b} can form together a substituted or unsubstituted carbocyclic or heterocyclic ring.

Preferably, R^{9a}, R^{10a}, R^{9b} and R^{10b} each independently represents hydrogen, a substituted or unsubstituted carbocyclic group having 5 to 18 ring carbon atoms, a substituted or unsubstituted heterocyclic group having 5 to 18 ring atoms, or CN, or

two adjacent groups R^{9a}, and/or two adjacent groups R^{10a}, or two adjacent groups R^{9b}, and/or two adjacent groups R^{10b} can form together a substituted or unsubstituted carbocyclic or heterocyclic ring.

More preferably, R^{9a}, R^{10a}, R^{9b} and R^{10b} each independently represents hydrogen, a substituted or unsubstituted phenyl group, a substituted or unsubstituted biphenyl group, a substituted or unsubstituted terphenyl group, a substituted or unsubstituted triphenylenyl group, a substituted or unsubstituted naphthyl group, a substituted or unsubstituted phenanthrenyl group, a substituted or unsubstituted dibenzofuranyl group, or a substituted or unsubstituted dibenzothiophenyl group, or

two adjacent groups R^{9a}, and/or two adjacent groups R^{10a}, or two adjacent groups R^{9b}, and/or two adjacent groups R^{10b} can form, together with the phenyl group to which R^{9a}, R^{10a}, R^{9b} and R^{10b} are attached, a substituted or unsubstituted dibenzofuranyl group, a substituted or unsubstituted dibenzothiophenyl group, or a substituted or unsubstituted fluorenyl group.

Most preferably, R^{9a}, R^{10a}, R^{9b} and R^{10b} each independently represents hydrogen, an unsubstituted phenyl group, an unsubstituted biphenyl group, an unsubstituted dibenzofuranyl group, or an unsubstituted dibenzothiophenyl group, or

two adjacent groups R^{9a}, and/or two adjacent groups R^{10a}, or two adjacent groups R^{9b}, and/or two adjacent groups R^{10b} can form, together with the phenyl group to which R^{9a}, R^{10a}, R^{9b} and R^{10b} are attached, a substituted or unsubstituted dibenzofuranyl group, a substituted or unsubstituted dibenzothiophenyl group, or a substituted or unsubstituted fluorenyl group.

Further most preferably, R^{9a}, R^{10a}, R^{9b} and R^{10b} each independently represents hydrogen, an unsubstituted phenyl group, or an unsubstituted biphenyl group, or

two adjacent groups R^{9a}, and/or two adjacent groups R^{10a}, or two adjacent groups R^{9b}, and/or two adjacent groups R^{10b} can form, together with the phenyl group to which R^{9a}, R^{10a}, R^{9b} and R^{10b} are attached, an unsubstituted dibenzofuranyl group, an unsubstituted dibenzothiophenyl group, or an unsubstituted 9,9-diphenylfluorenyl group.

Still further most preferably, R^{9a}, R^{10a}, R^{9b} and R^{10b} are hydrogen.

R^{11a} and R^{11b}

R^{11a} and R^{11b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN, preferably hydrogen, a substituted or unsubstituted alkyl group having 1 to 25 carbon atoms, a substituted or unsubstituted carbocyclic group having 5 to 18 ring carbon atoms, or CN,

### Or

two groups R^{11a} on adjacent rings A or two groups R^{11b} on adjacent rings B can form together an unsubstituted carbocyclic ring or a carbocyclic ring substituted by one or more heteroatom-free substituents, and/or

two adjacent groups R^{11a} on one of the rings A or two adjacent groups R^{11b} on one of the rings B can form together a substituted or unsubstituted carbocyclic ring.

The carbocyclic ring formed by two groups R^{11a} on adjacent rings A or two groups R^{11b} on adjacent rings B is preferably a five membered carbocyclic ring or a six membered carbocyclic ring.

Examples for heteroatom-free substituents are carbocyclic substituents, such as phenyl group, naphthyl group or biphenyl group; C₁-C₈ alkyl groups, such as methyl group, ethyl group, n-propyl group, isopropyl group, n-butyl group, s-butyl group, isobutyl group, or t-butyl group; and/or

two heteroatom-free substituents together form a carbocycle, which is saturated or unsaturated and unsubstituted or substituted by one or more heteroatom-free substituents. Preferably, the carbocycle is a saturated or unsaturated five membered ring or a saturated or unsaturated six membered ring.

Examples for suitable groups

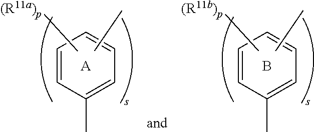

are biphenylene groups (s=2), which are unsubstituted or substituted by one or more residues R^{11a} or R^{11b}, or the following groups, wherein s is 2:

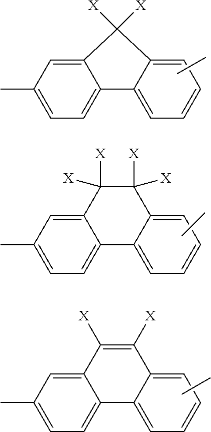

X═H or a heteroatom free substituent

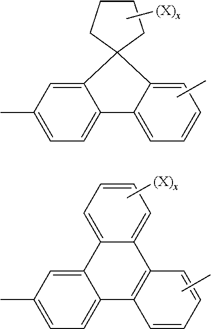

X═H or a heteroatom free substituent

wherein x is 0 to 8, preferably 0, 1, 2, 3 or 4, more preferably 0, 1 or 2; and/or two adjacent groups X form together a saturated or unsaturated and substituted or unsubstituted carbocyclic ring, preferably do not form together a ring.

Preferably, R^{11a} and R^{11b} each independently represents hydrogen, a substituted or unsubstituted carbocyclic group having 5 to 18 ring carbon atoms, a substituted or unsubstituted heterocyclic group having 5 to 18 ring atoms, or CN, preferably hydrogen, a substituted or unsubstituted carbocyclic group having 5 to 18 ring carbon atoms, or CN, or two groups R^{11a} on adjacent rings A or two groups R^{11b} on adjacent rings B can form together an unsubstituted carbocyclic ring or a carbocyclic ring substituted by one or more heteroatom-free substituents, and/or

two adjacent groups R^{11a} on one of the rings A or two adjacent groups R^{11b} on one of the rings B can form together a substituted or unsubstituted carbocyclic ring, preferably a substituted or unsubstituted phenyl ring.

More preferably, R^{11a} and R^{11b} each independently represents hydrogen, an unsubstituted phenyl group, an unsubstituted biphenyl group, an unsubstituted pyridinyl group, an unsubstituted dibenzofuranyl group, an unsubstituted quinolinyl group, or an unsubstituted dibenzothiophenyl group, preferably hydrogen, an unsubstituted phenyl group, or an unsubstituted biphenyl group, or two groups R^{11a} on adjacent rings A or two groups R^{11b} on adjacent rings B can form together an unsubstituted carbocyclic ring or a carbocyclic ring substituted by one or more heteroatom-free substituents, and/or

two adjacent groups R^{11a} on one of the rings A or two adjacent groups R^{11b} on one of the rings B can form together a substituted or unsubstituted carbocyclic ring, preferably a substituted or unsubstituted phenyl ring.

Most preferably, R^{11a} and R^{11b} each independently represents hydrogen, an unsubstituted phenyl group, or an unsubstituted pyridinyl group, preferably hydrogen or an unsubstituted phenyl group,

or

two groups R^{11a} on adjacent rings A or two groups R^{11b} on adjacent rings B can form together an unsubstituted carbocyclic ring or a carbocyclic ring substituted by one or more heteroatom-free substituents, and/or

two adjacent groups R^{11a} on one of the rings A or two adjacent groups R^{11b} on one of the rings B can form together a substituted or unsubstituted phenyl ring, preferably an unsubstituted phenyl ring.

Further most preferably, R^{11a} and R^{11b} are hydrogen.

X^{a1}, X^{a2}, X^{a3}, X^{b1}, X^{b2} and X^{b3}

X^{a1}, X^{a2}, X^{a3}, X^{b1}, X^{b2} and X^{b3} each independently represents N or CR¹², wherein at least two of X^{a1}, X^{a2} and X^{a3} and at least two of X^{b1}, X^{b2} and X^{b3} are N;

R¹² represents in each case independently hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN; preferably hydrogen, an unsubstituted aryl group having 6 to 18 ring carbon atoms or an unsubstituted heteroaryl group having 5 to 18 ring atoms; more preferably hydrogen or unsubstituted phenyl.

Preferably, X^{a1}, X^{a2}, X^{a3}, X^{b1}, X^{b2} and X^{b3} each represents N.

Y^{a} and Y^{b}

Y^{a} and r each independently represents S or O.

n, p, q, r and s

n is 1 or 2, preferably 1;

q and r are each independently 1, 2, 3, 4 or 5, preferably 1 or 2, more preferably 1;

p is 1, 2, 3 or 4, preferably 1 or 2, more preferably 1; and

s is 2, 3 or 4, preferably 2 or 3, more preferably 2.

### Groups

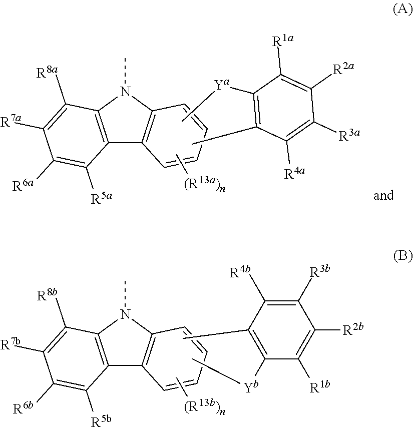

The group (A) is preferably represented by the following formula:

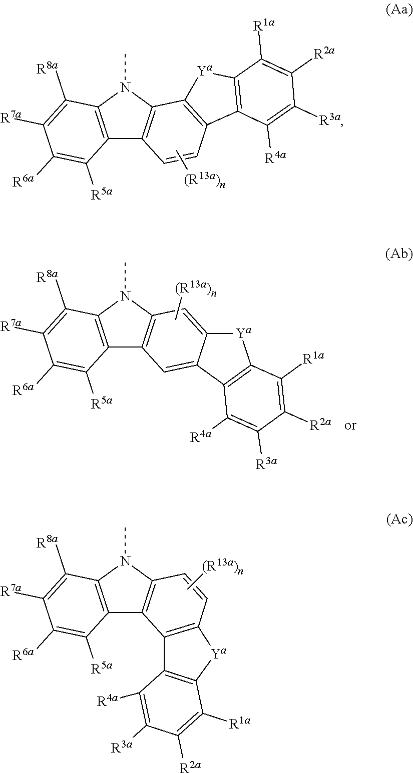

and the group (B) is preferably represented by the following formula:

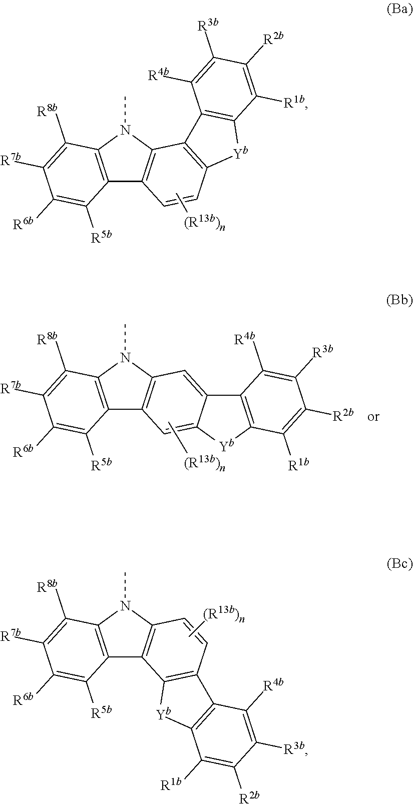

wherein Y^{a}, Y^{b}, R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} and n are defined above, and the dotted line is a bonding site to the remaining part of the polycyclic compound of formula (Ia) or (Ib). Preferably, R^{1a}, R^{2a}, R^{3a}, R^{4a}, R^{5a}, R^{8a}, R^{7a}, R^{8a}, R^{1b}, R^{2b}, R^{3b}, R^{4b}, R^{5b}, R^{6b}, R^{7b}, R^{8b}, R^{13a} and R^{13b} are hydrogen or phenyl, more preferably hydrogen.

It has been found by the inventors that the inventive polycyclic compounds are particularly suitable for organic electroluminescence devices having high external quantum efficiencies (EQE), long lifetime and/or low driving voltages.

### Compounds of Formula (Ia) or (Ib)

Preferably, the compounds of formula (Ia) or (Ib) are represented by the following formula (Iaa), (Iba), (Ica) or (Ida):

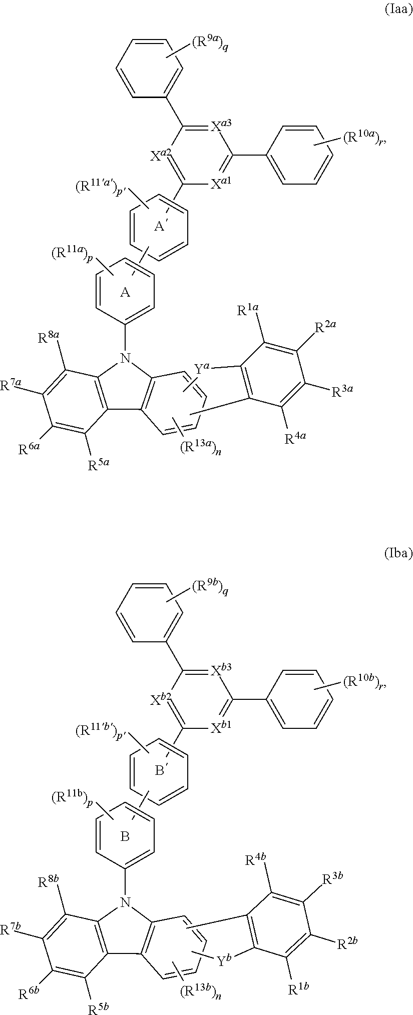

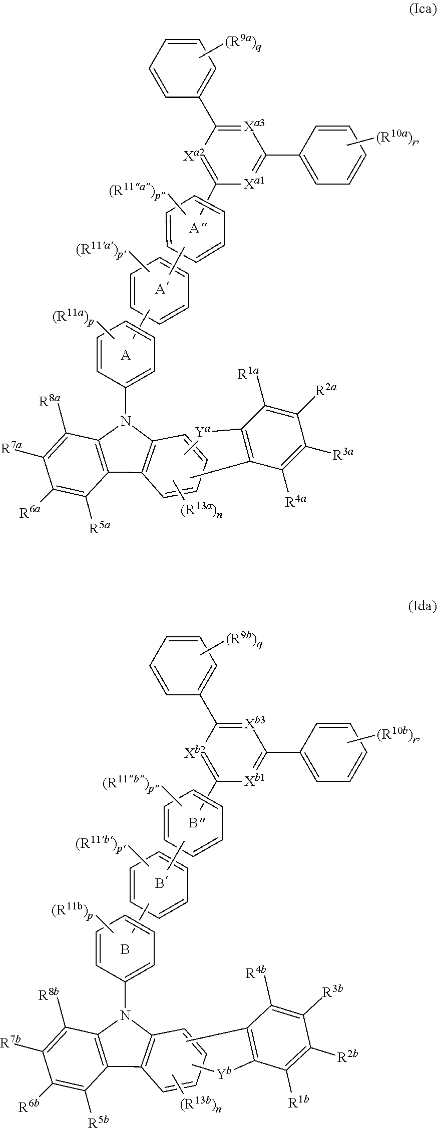

wherein the groups, residues and indices R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a}, R^{13b}, R^{9a}, R^{10a}, R^{11a}, X^{a1}, X^{a2}, X^{a3}, Y^{a}, R^{9b}, R^{10b}, R^{11b}, X^{b1}, X^{b2}, X^{b3}, Y^{b}, n, q, r and p are defined above and below, and R^{11′a′}, p′ and R^{11′b′} and R^{11″a″}, p″ and R^{11″b″} are each independently defined as R^{11a}, p and R^{11b}.

Preferably, R^{11a}, R^{11′a′}, R^{11″a″}, R^{11″b″} and R^{11b} each independently represents a hydrogen, an unsubstituted phenyl group, or an unsubstituted pyridinyl group, preferably hydrogen or an unsubstituted phenyl group, more preferably hydrogen.

or

two groups R^{11a} and R^{11′a′} on adjacent rings A and A′, two groups R^{11b} and R^{11′b′} on adjacent rings B and B′, two groups R^{11′a′} and R^{11″a″} on adjacent rings A′ and A″, or two groups R^{11′b′} and R^{11″b″} on adjacent rings B′ and B″ can form together an unsubstituted carbocyclic ring or a carbocyclic ring substituted by one or more heteroatom-free substituents, and/or

two adjacent groups R^{11a}, R^{11′a′} or R^{11″a″} on one of the rings A, A′ or A″ or two adjacent groups R^{11′b′}, R^{11″b″} or R^{11b} on one of the rings B, B′ or B″ can form together a substituted or unsubstituted phenyl ring, preferably an unsubstituted phenyl ring.

More preferably, the compounds of formula (Ia) or (Ib) are represented by the formula (Iaa) or (Iba).

Further preferably, the compounds of formula (Iaa), (Iba), (Ica) or (Ida) are represented by the following formula (Iaa1), (Iba1), (Ica1) or (Ida1):

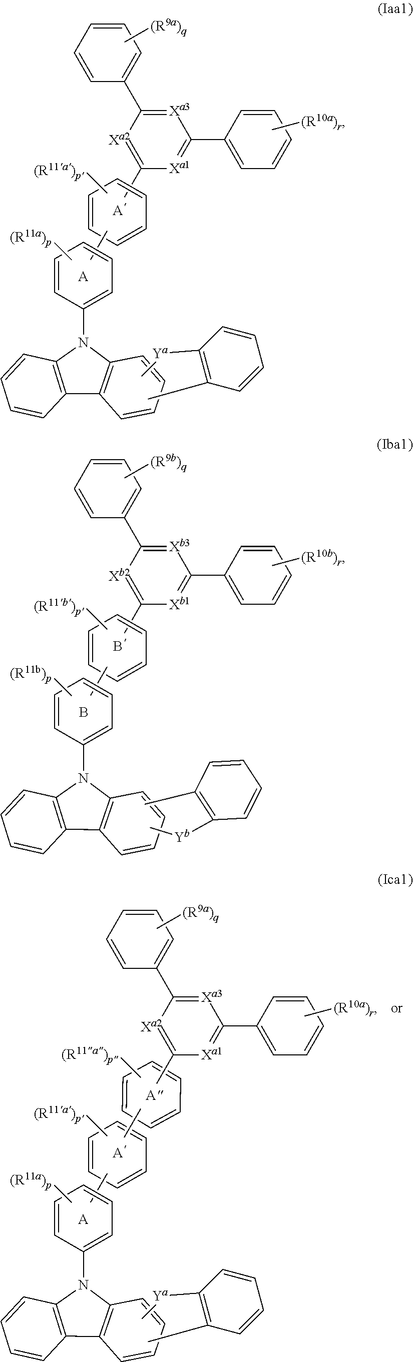

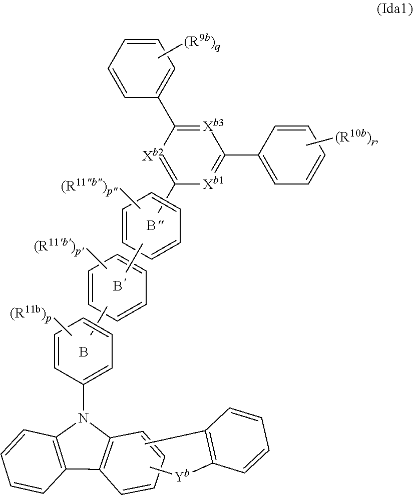

wherein the groups, residues and indices R^{9a}, R^{10a}, R^{11a}, X^{a1}, X^{a2}, X^{a3}, Y^{a}, R^{9b}, R^{10b}, R^{11b}, X^{b1}, X^{b2}, X^{b3}, r, q, r and p are defined above and below, and R^{11′a′}, p′ and R^{11′b′} and R^{11″a″}, p″ and R^{11″b″} are each independently defined as R^{11a}, p and R^{11b}.

Preferably, R^{11a}, R^{1l′a′}, R^{11″a″}, R^{11′b}′, R^{11″b″} and R^{11b} each independently represents hydrogen, an unsubstituted phenyl group or an unsubstituted pyridinyl group, more preferably hydrogen or an unsubstituted phenyl group, and still more preferably hydrogen,

or

two groups R^{11a} and R^{11′a′} on adjacent rings A and A′, two groups R^{11b} and R^{11′b′} on adjacent rings B and B′, two groups R^{11′a′} and R^{11″a″} on adjacent rings A′ and A″, or two groups R^{11′b′} and R^{11″b″} on adjacent rings B′ and B″ can form together an unsubstituted carbocyclic ring or a carbocyclic ring substituted by one or more heteroatom-free substituents, and/or

two adjacent groups R^{11a}, R^{11′a′} or R^{11″a″} on one of the rings A, A′ or A″ or two adjacent groups R^{11b′}, R^{11″b″} or R^{11b} on one of the rings B, B′ or B″ can form together a substituted or unsubstituted phenyl ring, preferably an unsubstituted phenyl ring.

Further more preferred are the compounds represented by the formula (Iaa1) or (Iba1).

Further more preferably, the compounds of formula (Iaa1), (Iba1), (Ica1) or (Ida1) are represented by the following formula (Iaa1a), (Iba1a), (Ica1a) or (Ida1a):

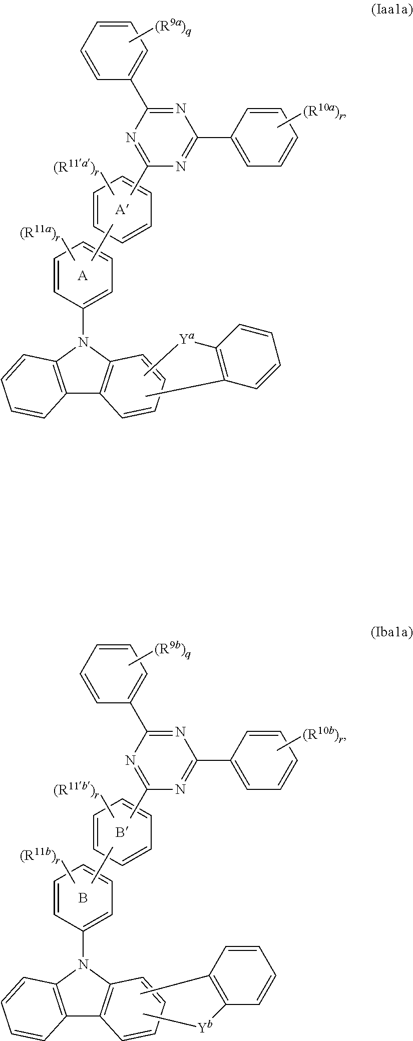

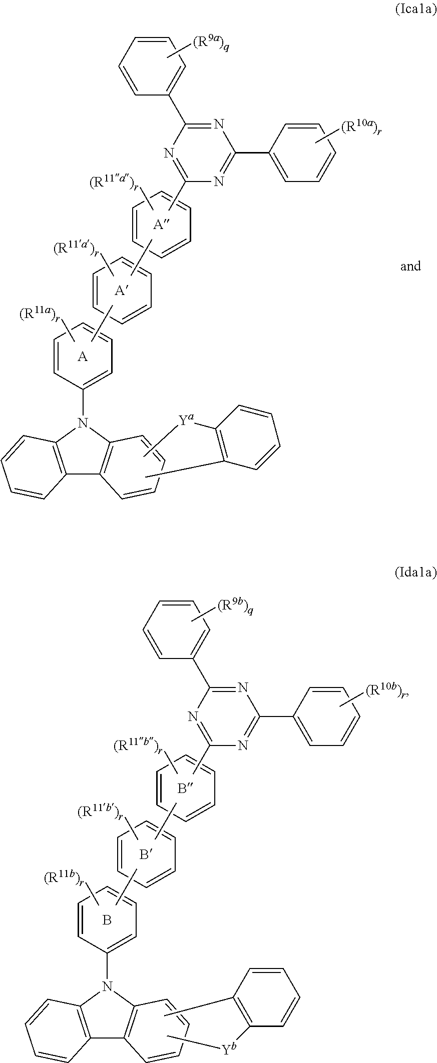

wherein the groups, residues and indices R^{9a}, R^{10a}, R^{11a}, Y^{a}, R^{9b}, R^{10b}, R^{11b}, Y^{b}, q, r and p are defined above and below, and R^{11′a′}, p′ and R^{11b′} and R^{11″a″}, p″ and R^{11″b″} are each independently defined as R^{11a}, p and R^{11b}.

Preferably, R^{11a}, R^{11′a′}, R^{11″a″}, R^{11′b′}, R^{11″b″} and R^{11b}, each independently represents a hydrogen, an unsubstituted phenyl group or an unsubstituted pyridinyl group, more preferably hydrogen or an unsubstituted phenyl group, and still more preferably hydrogen,

### Or

two groups R^{11a} and R^{11′a′} on adjacent rings A and A′, two groups R^{11b} and R^{11′b′} on adjacent rings B and B′, two groups R^{11′a′} and R^{11″a″} on adjacent rings A′ and A″, or two groups R^{11′b′} and R^{11″b″} on adjacent rings B′ and B″ can form together an unsubstituted carbocyclic ring or a carbocyclic ring substituted by one or more heteroatom-free substituents, and/or

two adjacent groups R^{11a}, R^{11′a′ or R}^{11″a″} on one of the rings A, A′ or A″ or two adjacent groups R^{11′b′}, R^{11″b″} or R^{11b} on one of the rings B, B′ or B″ can form together a substituted or unsubstituted phenyl ring, preferably an unsubstituted phenyl ring.

Below, examples for compounds of formulae (Ia) and (Ib) are given:

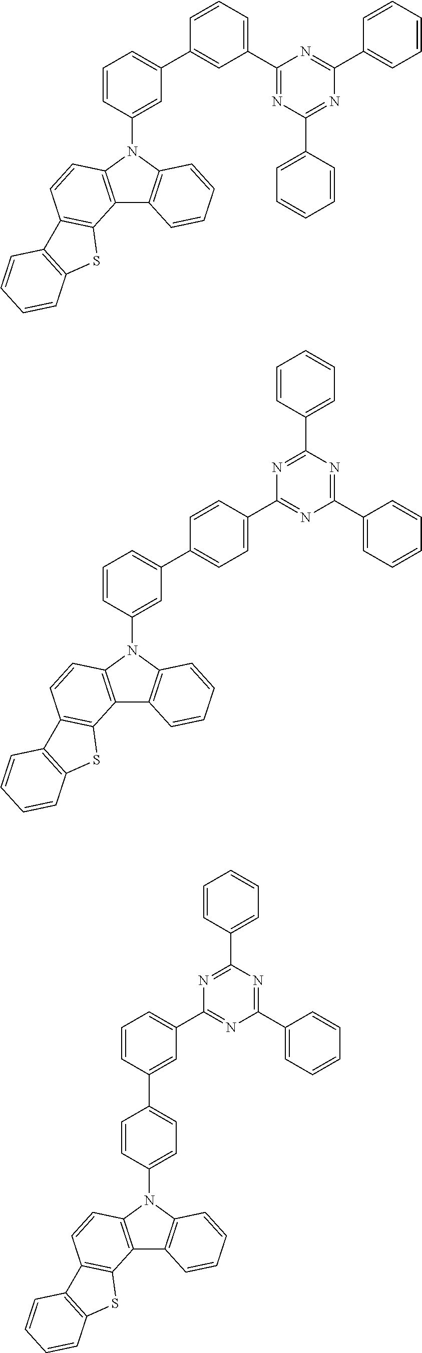 [SMILES: c1ccc(-c2nc(-c3ccccc3)nc(-c3cccc(-c4cccc(-n5c6ccccc6c6c7sc8ccccc8c7ccc65)c4)c3)n2)cc1 ; c1ccc(-c2nc(-c3ccccc3)nc(-c3ccc(-c4cccc(-n5c6ccccc6c6c7sc8ccccc8c7ccc65)c4)cc3)n2)cc1 ; c1ccc(-c2nc(-c3ccccc3)nc(-c3cccc(-c4ccc(-n5c6ccccc6c6c7sc8ccccc8c7ccc65)cc4)c3)n2)cc1]

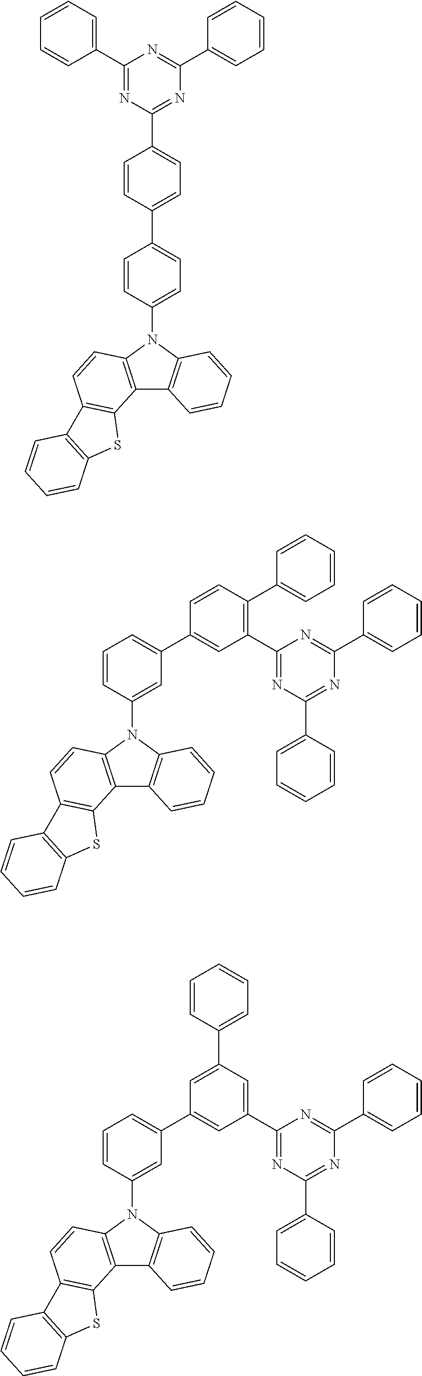 [SMILES: c1ccc(-c2nc(-c3ccccc3)nc(-c3ccc(-c4ccc(-n5c6ccccc6c6c7sc8ccccc8c7ccc65)cc4)cc3)n2)cc1 ; c1ccc(-c2nc(-c3ccccc3)nc(-c3cc(-c4cccc(-n5c6ccccc6c6c7sc8ccccc8c7ccc65)c4)ccc3-c3ccccc3)n2)cc1 ; c1ccc(-c2cc(-c3cccc(-n4c5ccccc5c5c6sc7ccccc7c6ccc54)c3)cc(-c3nc(-c4ccccc4)nc(-c4ccccc4)n3)c2)cc1]

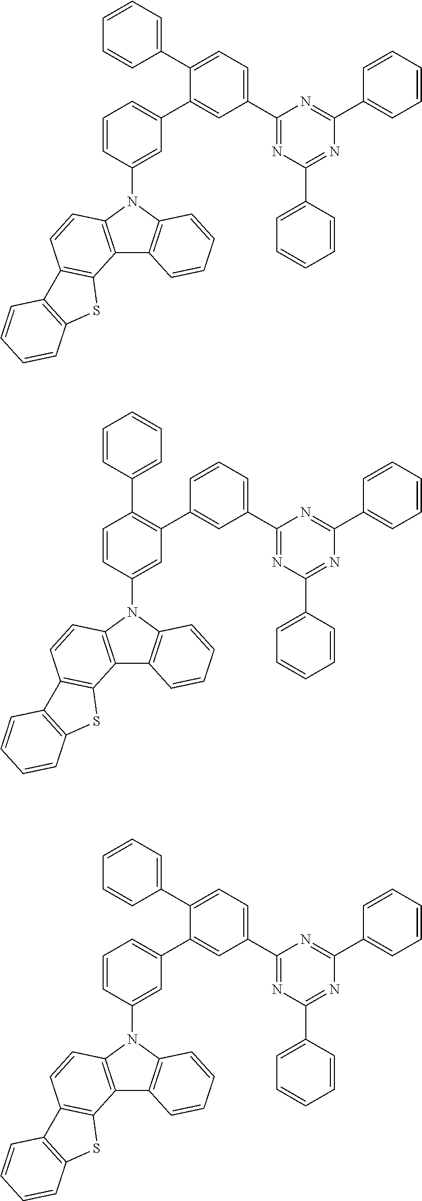 [SMILES: c1ccc(-c2nc(-c3ccccc3)nc(-c3ccc(-c4ccccc4)c(-c4cccc(-n5c6ccccc6c6c7sc8ccccc8c7ccc65)c4)c3)n2)cc1 ; c1ccc(-c2nc(-c3ccccc3)nc(-c3cccc(-c4cc(-n5c6ccccc6c6c7sc8ccccc8c7ccc65)ccc4-c4ccccc4)c3)n2)cc1 ; c1ccc(-c2nc(-c3ccccc3)nc(-c3ccc(-c4ccccc4)c(-c4cccc(-n5c6ccccc6c6c7sc8ccccc8c7ccc65)c4)c3)n2)cc1]

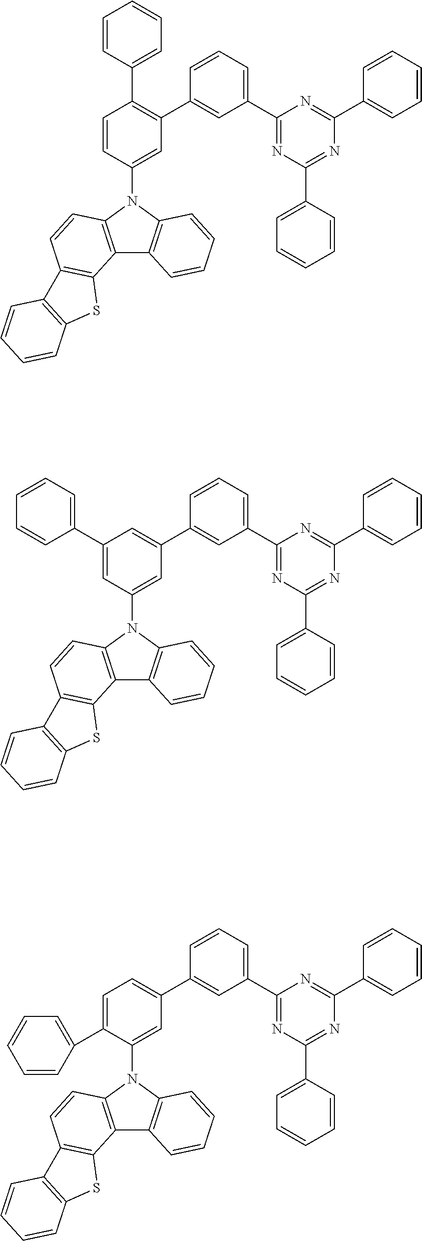 [SMILES: c1ccc(-c2nc(-c3ccccc3)nc(-c3cccc(-c4cc(-n5c6ccccc6c6c7sc8ccccc8c7ccc65)ccc4-c4ccccc4)c3)n2)cc1 ; c1ccc(-c2cc(-c3cccc(-c4nc(-c5ccccc5)nc(-c5ccccc5)n4)c3)cc(-n3c4ccccc4c4c5sc6ccccc6c5ccc43)c2)cc1 ; c1ccc(-c2nc(-c3ccccc3)nc(-c3cccc(-c4ccc(-c5ccccc5)c(-n5c6ccccc6c6c7sc8ccccc8c7ccc65)c4)c3)n2)cc1]

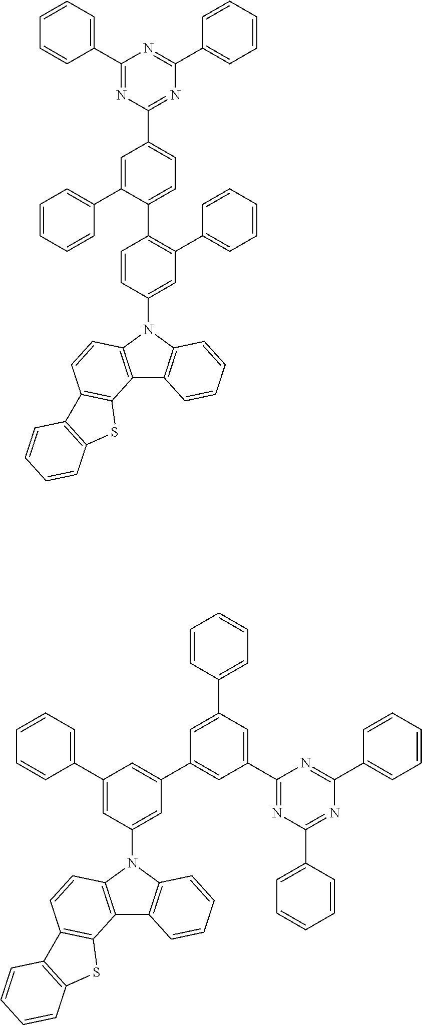 [SMILES: c1ccc(-c2nc(-c3ccccc3)nc(-c3ccc(-c4ccc(-n5c6ccccc6c6c7sc8ccccc8c7ccc65)cc4-c4ccccc4)c(-c4ccccc4)c3)n2)cc1 ; c1ccc(-c2cc(-c3cc(-c4ccccc4)cc(-n4c5ccccc5c5c6sc7ccccc7c6ccc54)c3)cc(-c3nc(-c4ccccc4)nc(-c4ccccc4)n3)c2)cc1]

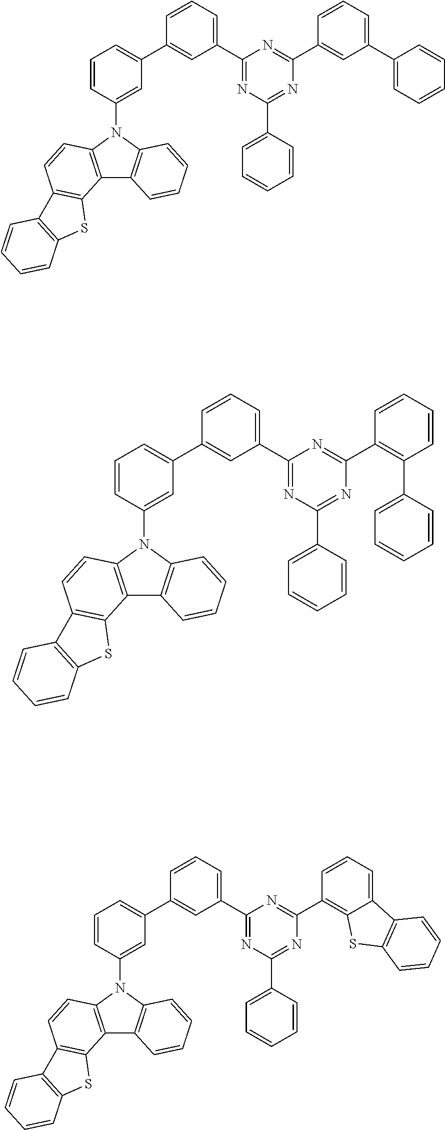 [SMILES: c1ccc(-c2cccc(-c3nc(-c4ccccc4)nc(-c4cccc(-c5cccc(-n6c7ccccc7c7c8sc9ccccc9c8ccc76)c5)c4)n3)c2)cc1 ; c1ccc(-c2nc(-c3cccc(-c4cccc(-n5c6ccccc6c6c7sc8ccccc8c7ccc65)c4)c3)nc(-c3ccccc3-c3ccccc3)n2)cc1 ; c1ccc(-c2nc(-c3cccc(-c4cccc(-n5c6ccccc6c6c7sc8ccccc8c7ccc65)c4)c3)nc(-c3cccc4c3sc3ccccc34)n2)cc1]

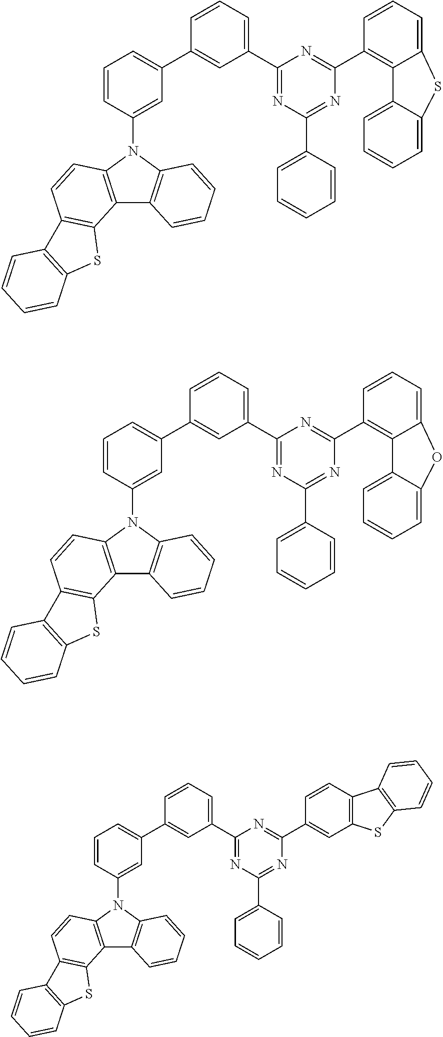 [SMILES: c1ccc(-c2nc(-c3cccc(-c4cccc(-n5c6ccccc6c6c7sc8ccccc8c7ccc65)c4)c3)nc(-c3cccc4sc5ccccc5c34)n2)cc1 ; c1ccc(-c2nc(-c3cccc(-c4cccc(-n5c6ccccc6c6c7sc8ccccc8c7ccc65)c4)c3)nc(-c3cccc4oc5ccccc5c34)n2)cc1 ; c1ccc(-c2nc(-c3cccc(-c4cccc(-n5c6ccccc6c6c7sc8ccccc8c7ccc65)c4)c3)nc(-c3ccc4c(c3)sc3ccccc34)n2)cc1]

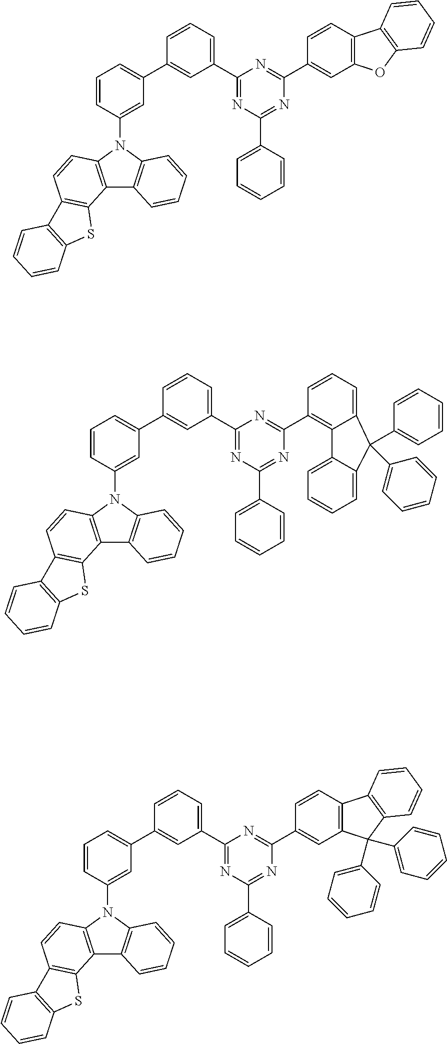 [SMILES: c1ccc(-c2nc(-c3cccc(-c4cccc(-n5c6ccccc6c6c7sc8ccccc8c7ccc65)c4)c3)nc(-c3ccc4c(c3)oc3ccccc34)n2)cc1 ; c1ccc(-c2nc(-c3cccc(-c4cccc(-n5c6ccccc6c6c7sc8ccccc8c7ccc65)c4)c3)nc(-c3cccc4c3-c3ccccc3C4(c3ccccc3)c3ccccc3)n2)cc1 ; c1ccc(-c2nc(-c3cccc(-c4cccc(-n5c6ccccc6c6c7sc8ccccc8c7ccc65)c4)c3)nc(-c3ccc4c(c3)C(c3ccccc3)(c3ccccc3)c3ccccc3-4)n2)cc1]

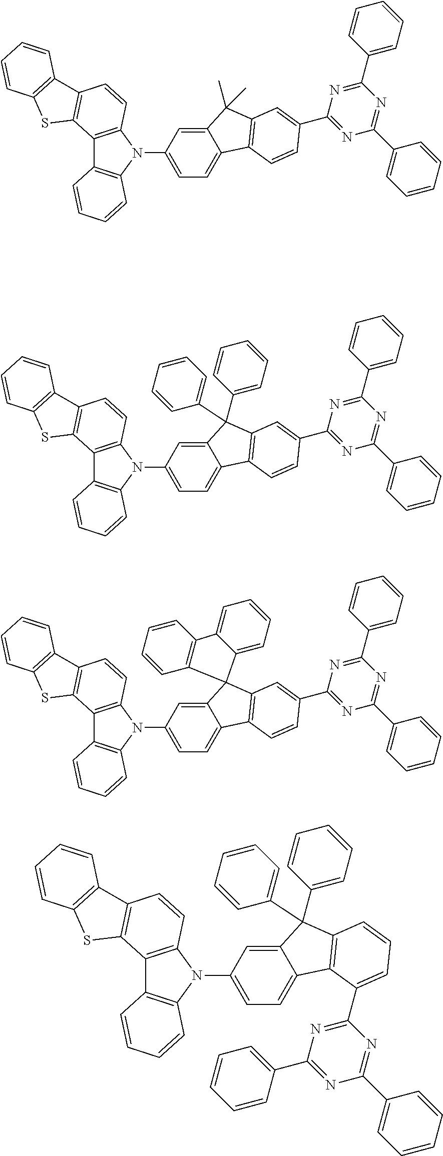 [SMILES: CC1(C)c2cc(-c3nc(-c4ccccc4)nc(-c4ccccc4)n3)ccc2-c2ccc(-n3c4ccccc4c4c5sc6ccccc6c5ccc43)cc21 ; c1ccc(-c2nc(-c3ccccc3)nc(-c3ccc4c(c3)C(c3ccccc3)(c3ccccc3)c3cc(-n5c6ccccc6c6c7sc8ccccc8c7ccc65)ccc3-4)n2)cc1 ; c1ccc(-c2nc(-c3ccccc3)nc(-c3ccc4c(c3)C3(c5ccccc5-c5ccccc53)c3cc(-n5c6ccccc6c6c7sc8ccccc8c7ccc65)ccc3-4)n2)cc1 ; c1ccc(-c2nc(-c3ccccc3)nc(-c3cccc4c3-c3ccc(-n5c6ccccc6c6c7sc8ccccc8c7ccc65)cc3C4(c3ccccc3)c3ccccc3)n2)cc1]

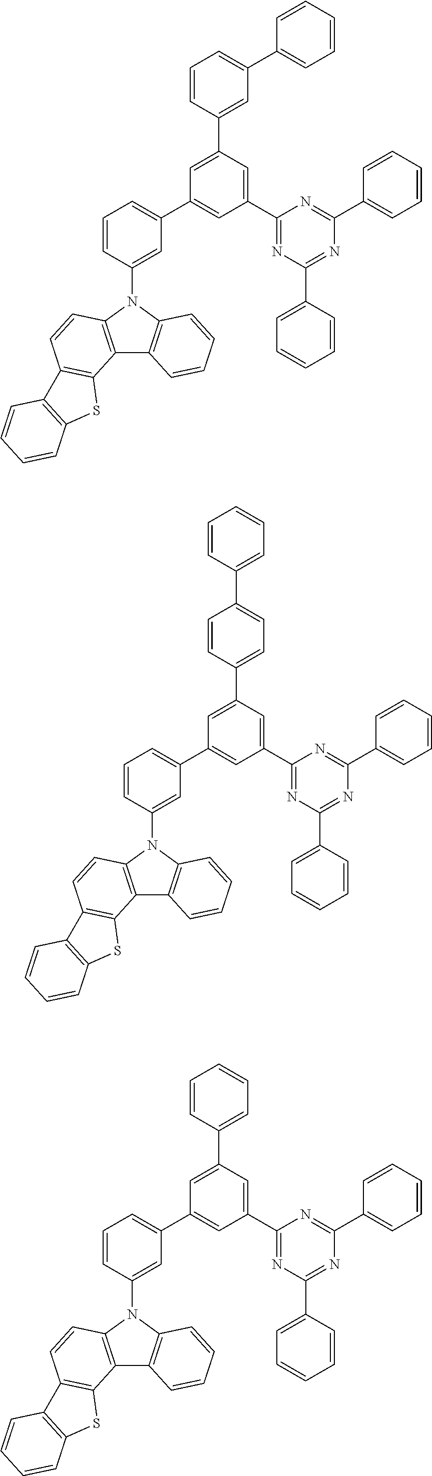 [SMILES: c1ccc(-c2cccc(-c3cc(-c4cccc(-n5c6ccccc6c6c7sc8ccccc8c7ccc65)c4)cc(-c4nc(-c5ccccc5)nc(-c5ccccc5)n4)c3)c2)cc1 ; c1ccc(-c2ccc(-c3cc(-c4cccc(-n5c6ccccc6c6c7sc8ccccc8c7ccc65)c4)cc(-c4nc(-c5ccccc5)nc(-c5ccccc5)n4)c3)cc2)cc1 ; c1ccc(-c2cc(-c3cccc(-n4c5ccccc5c5c6sc7ccccc7c6ccc54)c3)cc(-c3nc(-c4ccccc4)nc(-c4ccccc4)n3)c2)cc1]

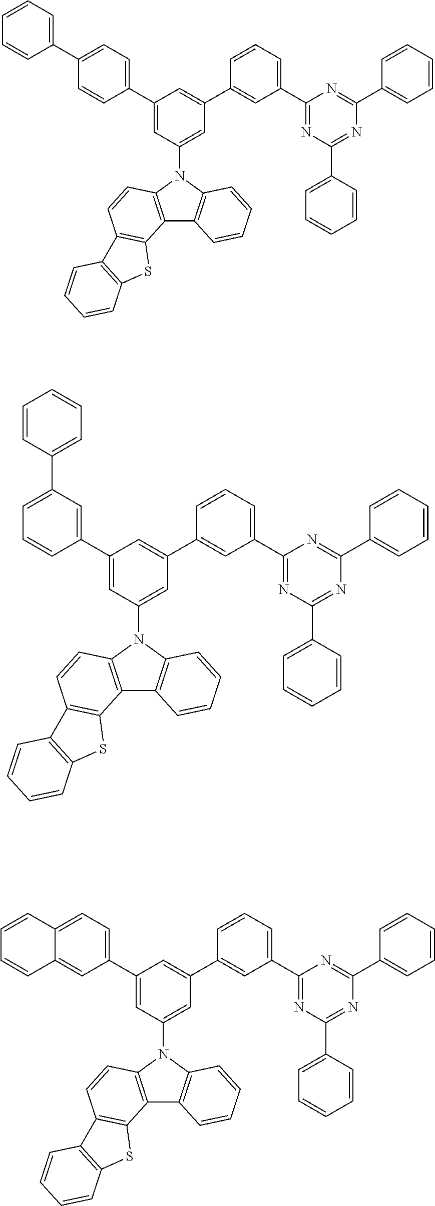 [SMILES: c1ccc(-c2ccc(-c3cc(-c4cccc(-c5nc(-c6ccccc6)nc(-c6ccccc6)n5)c4)cc(-n4c5ccccc5c5c6sc7ccccc7c6ccc54)c3)cc2)cc1 ; c1ccc(-c2cccc(-c3cc(-c4cccc(-c5nc(-c6ccccc6)nc(-c6ccccc6)n5)c4)cc(-n4c5ccccc5c5c6sc7ccccc7c6ccc54)c3)c2)cc1 ; c1ccc(-c2nc(-c3ccccc3)nc(-c3cccc(-c4cc(-c5ccc6ccccc6c5)cc(-n5c6ccccc6c6c7sc8ccccc8c7ccc65)c4)c3)n2)cc1]

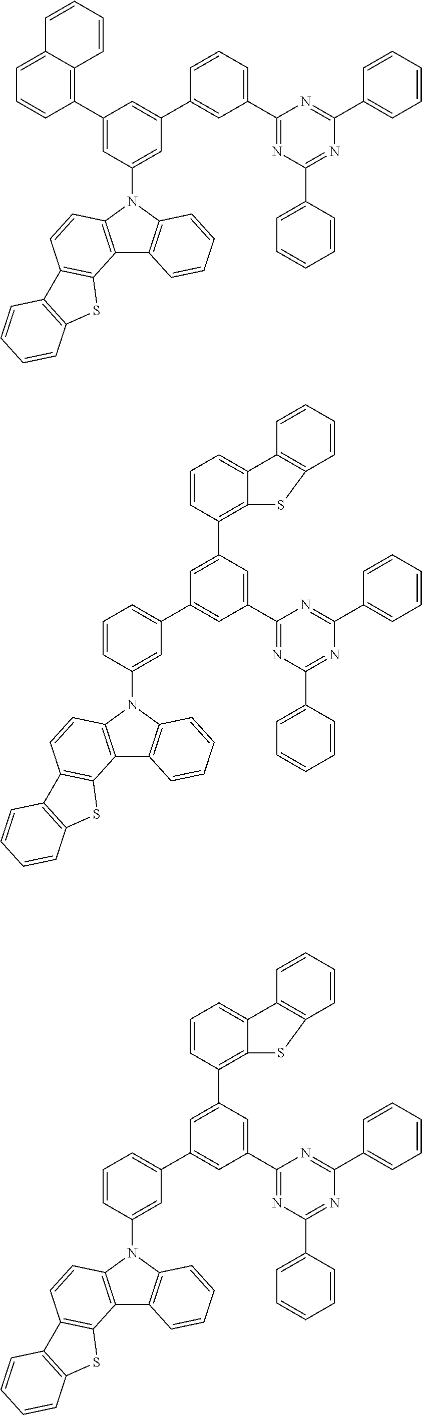 [SMILES: c1ccc(-c2nc(-c3ccccc3)nc(-c3cccc(-c4cc(-c5cccc6ccccc56)cc(-n5c6ccccc6c6c7sc8ccccc8c7ccc65)c4)c3)n2)cc1 ; c1ccc(-c2nc(-c3ccccc3)nc(-c3cc(-c4cccc(-n5c6ccccc6c6c7sc8ccccc8c7ccc65)c4)cc(-c4cccc5c4sc4ccccc45)c3)n2)cc1 ; c1ccc(-c2nc(-c3ccccc3)nc(-c3cc(-c4cccc(-n5c6ccccc6c6c7sc8ccccc8c7ccc65)c4)cc(-c4cccc5c4sc4ccccc45)c3)n2)cc1]

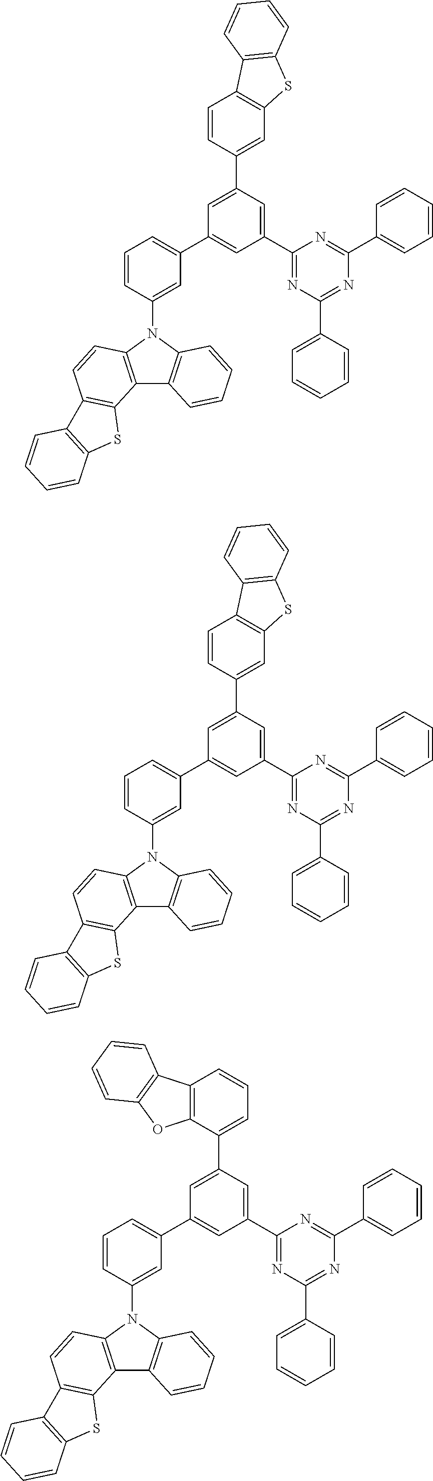 [SMILES: c1ccc(-c2nc(-c3ccccc3)nc(-c3cc(-c4cccc(-n5c6ccccc6c6c7sc8ccccc8c7ccc65)c4)cc(-c4ccc5c(c4)sc4ccccc45)c3)n2)cc1 ; c1ccc(-c2nc(-c3ccccc3)nc(-c3cc(-c4cccc(-n5c6ccccc6c6c7sc8ccccc8c7ccc65)c4)cc(-c4ccc5c(c4)sc4ccccc45)c3)n2)cc1 ; c1ccc(-c2nc(-c3ccccc3)nc(-c3cc(-c4cccc(-n5c6ccccc6c6c7sc8ccccc8c7ccc65)c4)cc(-c4cccc5c4oc4ccccc45)c3)n2)cc1]

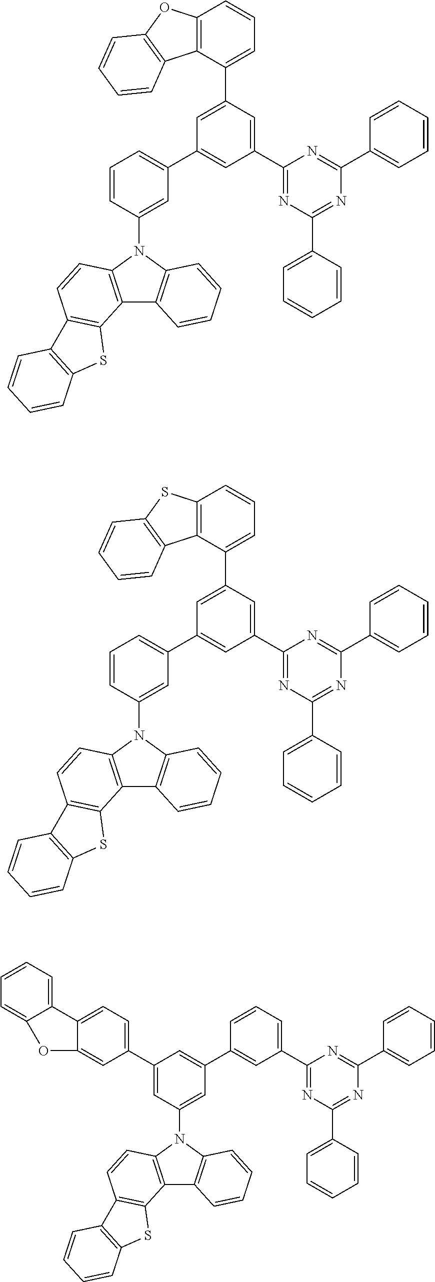 [SMILES: c1ccc(-c2nc(-c3ccccc3)nc(-c3cc(-c4cccc(-n5c6ccccc6c6c7sc8ccccc8c7ccc65)c4)cc(-c4cccc5oc6ccccc6c45)c3)n2)cc1 ; c1ccc(-c2nc(-c3ccccc3)nc(-c3cc(-c4cccc(-n5c6ccccc6c6c7sc8ccccc8c7ccc65)c4)cc(-c4cccc5sc6ccccc6c45)c3)n2)cc1 ; c1ccc(-c2nc(-c3ccccc3)nc(-c3cccc(-c4cc(-c5ccc6c(c5)oc5ccccc56)cc(-n5c6ccccc6c6c7sc8ccccc8c7ccc65)c4)c3)n2)cc1]

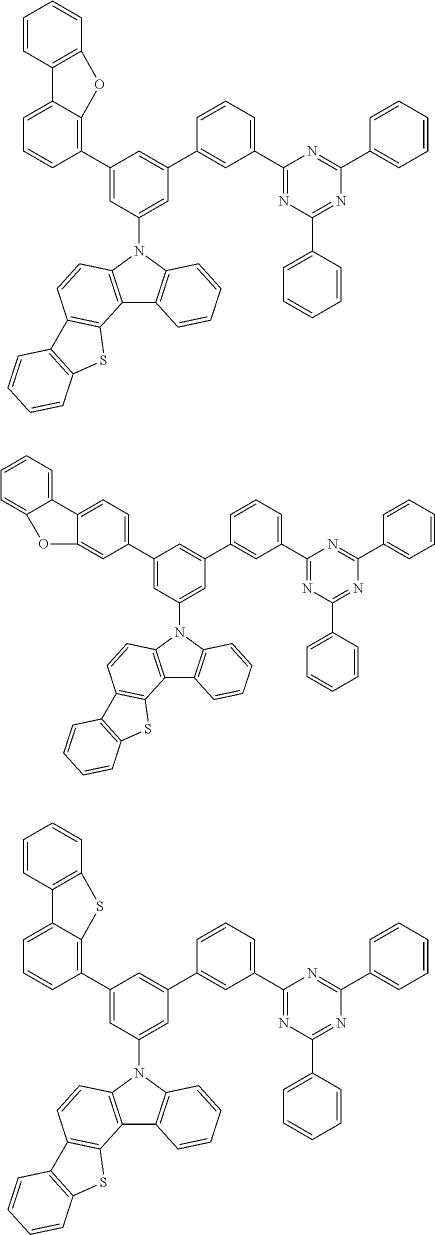

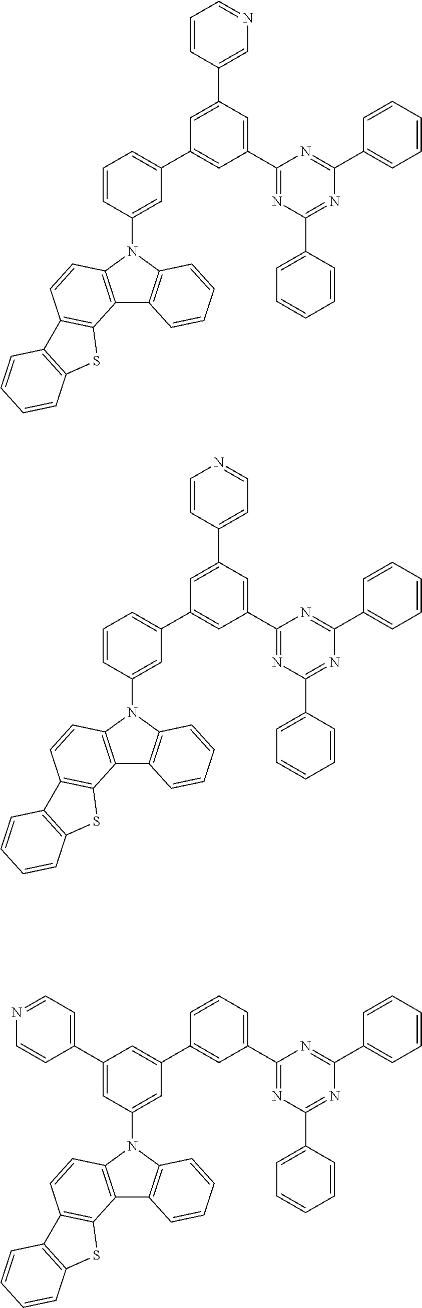 [SMILES: c1ccc(-c2nc(-c3ccccc3)nc(-c3cc(-c4cccnc4)cc(-c4cccc(-n5c6ccccc6c6c7sc8ccccc8c7ccc65)c4)c3)n2)cc1 ; c1ccc(-c2nc(-c3ccccc3)nc(-c3cc(-c4ccncc4)cc(-c4cccc(-n5c6ccccc6c6c7sc8ccccc8c7ccc65)c4)c3)n2)cc1 ; c1ccc(-c2nc(-c3ccccc3)nc(-c3cccc(-c4cc(-c5ccncc5)cc(-n5c6ccccc6c6c7sc8ccccc8c7ccc65)c4)c3)n2)cc1]

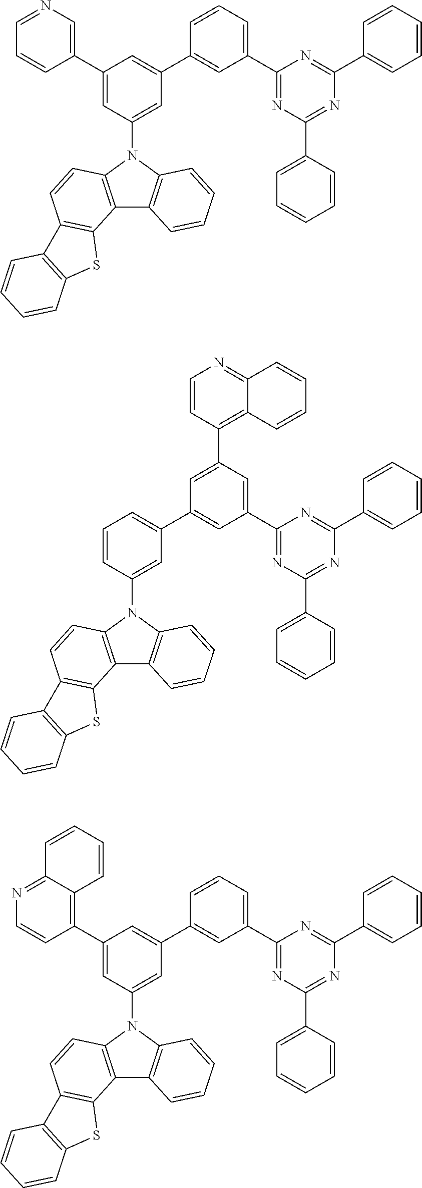 [SMILES: c1ccc(-c2nc(-c3ccccc3)nc(-c3cccc(-c4cc(-c5cccnc5)cc(-n5c6ccccc6c6c7sc8ccccc8c7ccc65)c4)c3)n2)cc1 ; c1ccc(-c2nc(-c3ccccc3)nc(-c3cc(-c4cccc(-n5c6ccccc6c6c7sc8ccccc8c7ccc65)c4)cc(-c4ccnc5ccccc45)c3)n2)cc1 ; c1ccc(-c2nc(-c3ccccc3)nc(-c3cccc(-c4cc(-c5ccnc6ccccc56)cc(-n5c6ccccc6c6c7sc8ccccc8c7ccc65)c4)c3)n2)cc1]

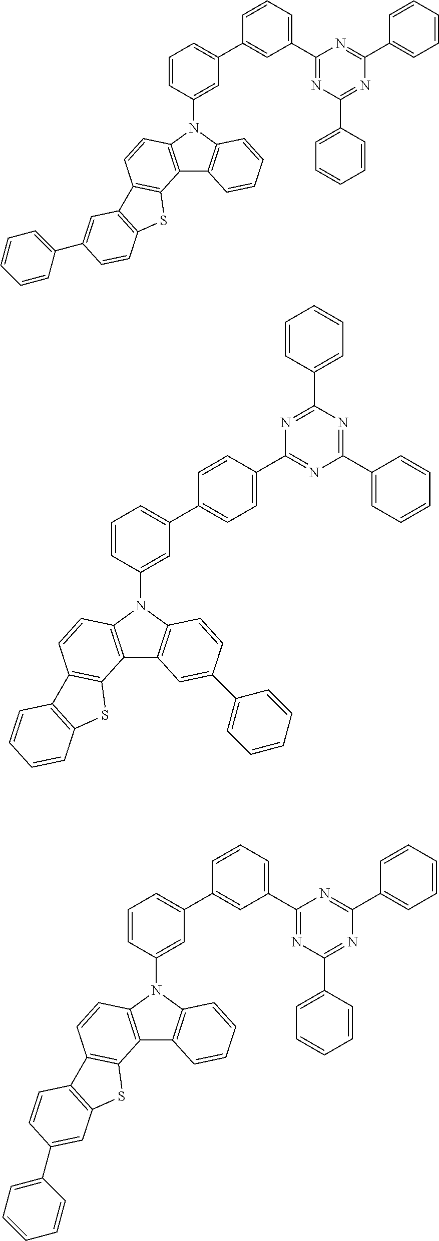 [SMILES: c1ccc(-c2ccc3sc4c(ccc5c4c4ccccc4n5-c4cccc(-c5cccc(-c6nc(-c7ccccc7)nc(-c7ccccc7)n6)c5)c4)c3c2)cc1 ; c1ccc(-c2ccc3c(c2)c2c4sc5ccccc5c4ccc2n3-c2cccc(-c3ccc(-c4nc(-c5ccccc5)nc(-c5ccccc5)n4)cc3)c2)cc1 ; c1ccc(-c2ccc3c(c2)sc2c3ccc3c2c2ccccc2n3-c2cccc(-c3cccc(-c4nc(-c5ccccc5)nc(-c5ccccc5)n4)c3)c2)cc1]

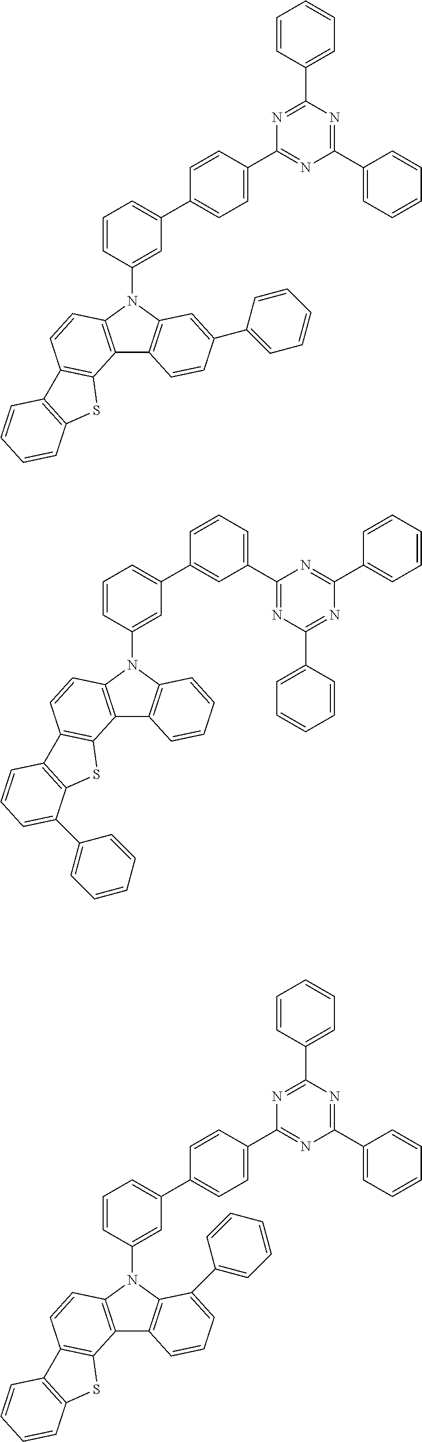 [SMILES: c1ccc(-c2ccc3c4c5sc6ccccc6c5ccc4n(-c4cccc(-c5ccc(-c6nc(-c7ccccc7)nc(-c7ccccc7)n6)cc5)c4)c3c2)cc1 ; c1ccc(-c2nc(-c3ccccc3)nc(-c3cccc(-c4cccc(-n5c6ccccc6c6c7sc8c(-c9ccccc9)cccc8c7ccc65)c4)c3)n2)cc1 ; c1ccc(-c2nc(-c3ccccc3)nc(-c3ccc(-c4cccc(-n5c6ccc7c8ccccc8sc7c6c6cccc(-c7ccccc7)c65)c4)cc3)n2)cc1]

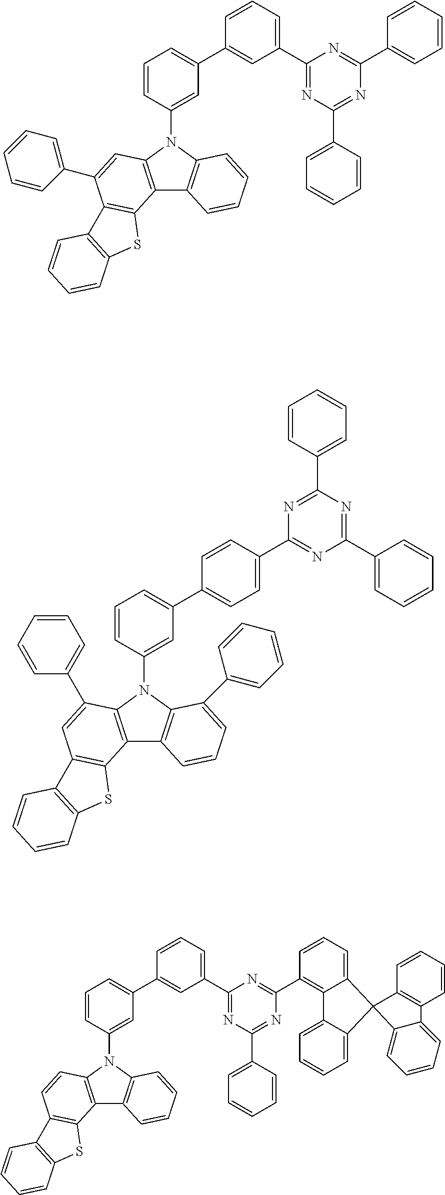 [SMILES: c1ccc(-c2nc(-c3ccccc3)nc(-c3cccc(-c4cccc(-n5c6ccccc6c6c7sc8ccccc8c7c(-c7ccccc7)cc65)c4)c3)n2)cc1 ; c1ccc(-c2nc(-c3ccccc3)nc(-c3ccc(-c4cccc(-n5c6c(-c7ccccc7)cccc6c6c7sc8ccccc8c7cc(-c7ccccc7)c65)c4)cc3)n2)cc1 ; c1ccc(-c2nc(-c3cccc(-c4cccc(-n5c6ccccc6c6c7sc8ccccc8c7ccc65)c4)c3)nc(-c3cccc4c3-c3ccccc3C43c4ccccc4-c4ccccc43)n2)cc1]

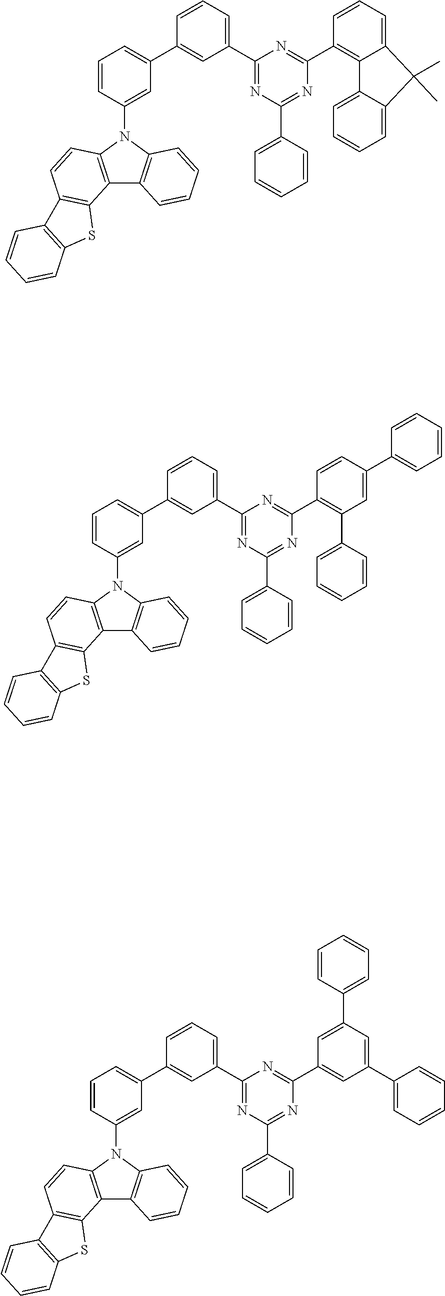 [SMILES: CC1(C)c2ccccc2-c2c(-c3nc(-c4ccccc4)nc(-c4cccc(-c5cccc(-n6c7ccccc7c7c8sc9ccccc9c8ccc76)c5)c4)n3)cccc21 ; c1ccc(-c2ccc(-c3nc(-c4ccccc4)nc(-c4cccc(-c5cccc(-n6c7ccccc7c7c8sc9ccccc9c8ccc76)c5)c4)n3)c(-c3ccccc3)c2)cc1 ; c1ccc(-c2cc(-c3ccccc3)cc(-c3nc(-c4ccccc4)nc(-c4cccc(-c5cccc(-n6c7ccccc7c7c8sc9ccccc9c8ccc76)c5)c4)n3)c2)cc1]

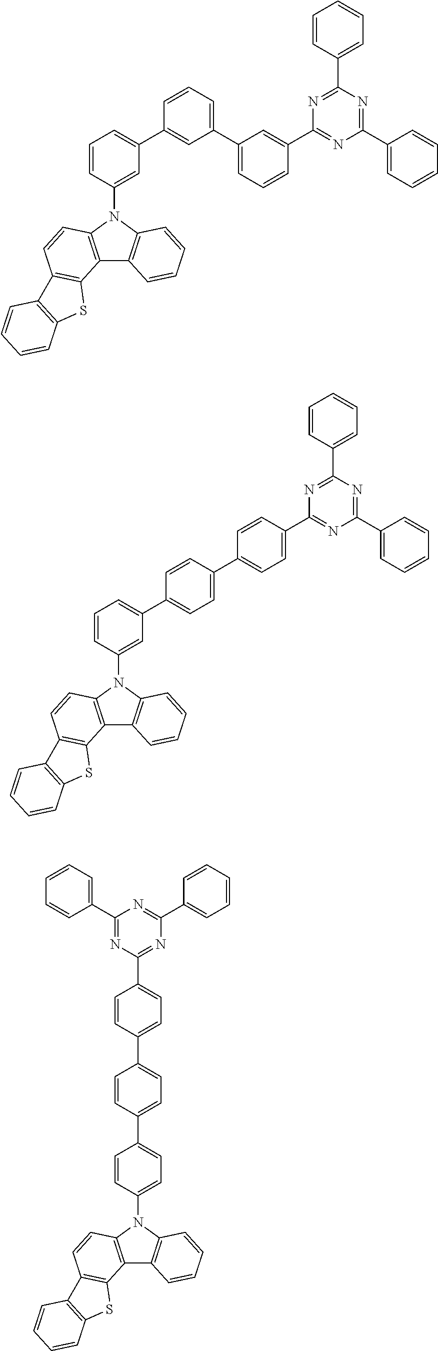 [SMILES: c1ccc(-c2nc(-c3ccccc3)nc(-c3cccc(-c4cccc(-c5cccc(-n6c7ccccc7c7c8sc9ccccc9c8ccc76)c5)c4)c3)n2)cc1 ; c1ccc(-c2nc(-c3ccccc3)nc(-c3ccc(-c4ccc(-c5cccc(-n6c7ccccc7c7c8sc9ccccc9c8ccc76)c5)cc4)cc3)n2)cc1 ; c1ccc(-c2nc(-c3ccccc3)nc(-c3ccc(-c4ccc(-c5ccc(-n6c7ccccc7c7c8sc9ccccc9c8ccc76)cc5)cc4)cc3)n2)cc1]

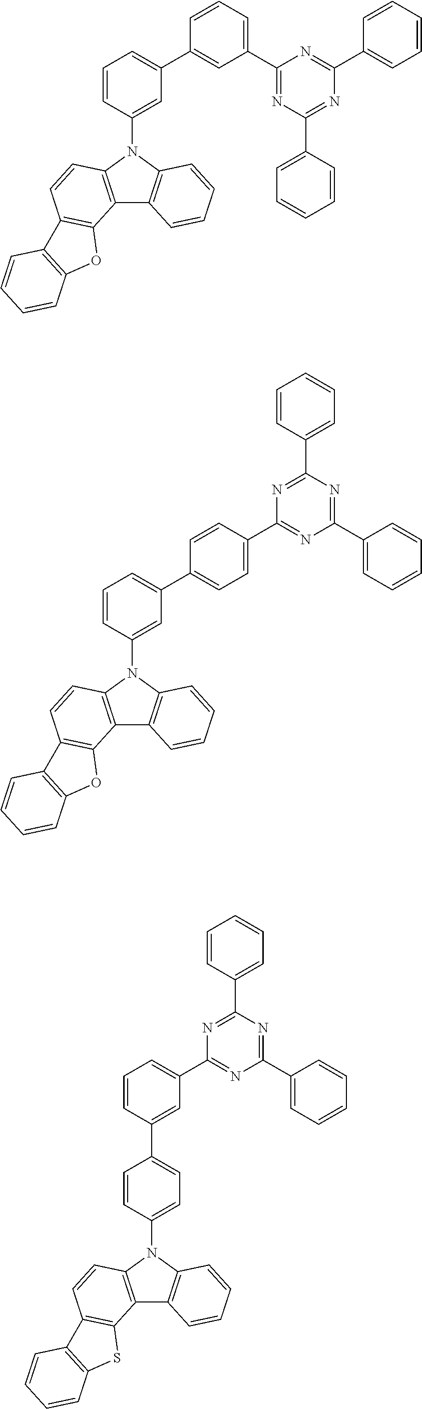 [SMILES: c1ccc(-c2nc(-c3ccccc3)nc(-c3cccc(-c4cccc(-n5c6ccccc6c6c7oc8ccccc8c7ccc65)c4)c3)n2)cc1 ; c1ccc(-c2nc(-c3ccccc3)nc(-c3ccc(-c4cccc(-n5c6ccccc6c6c7oc8ccccc8c7ccc65)c4)cc3)n2)cc1 ; c1ccc(-c2nc(-c3ccccc3)nc(-c3cccc(-c4ccc(-n5c6ccccc6c6c7sc8ccccc8c7ccc65)cc4)c3)n2)cc1]

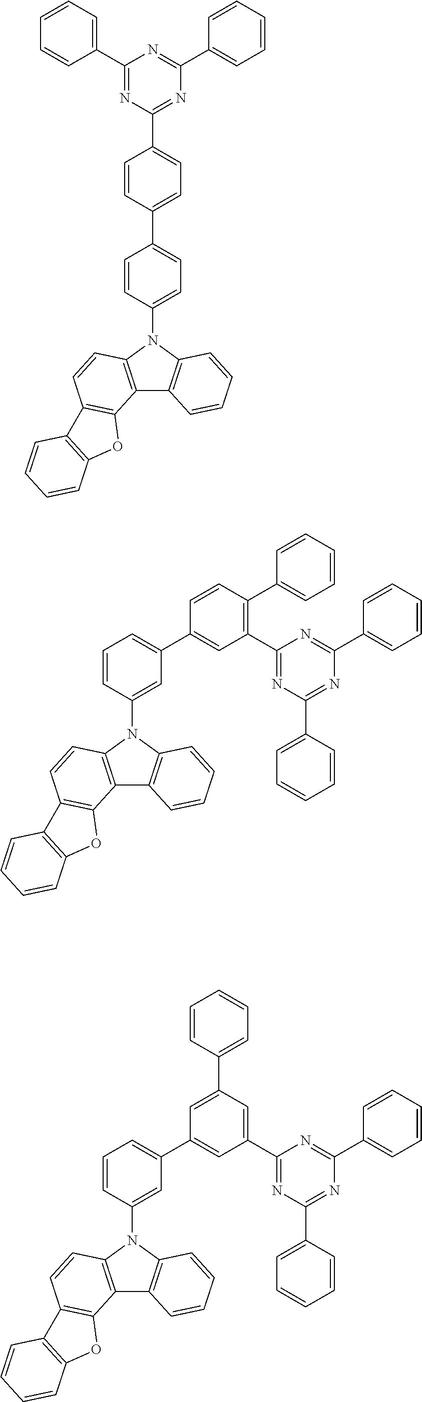 [SMILES: c1ccc(-c2nc(-c3ccccc3)nc(-c3ccc(-c4ccc(-n5c6ccccc6c6c7oc8ccccc8c7ccc65)cc4)cc3)n2)cc1 ; c1ccc(-c2nc(-c3ccccc3)nc(-c3cc(-c4cccc(-n5c6ccccc6c6c7oc8ccccc8c7ccc65)c4)ccc3-c3ccccc3)n2)cc1 ; c1ccc(-c2cc(-c3cccc(-n4c5ccccc5c5c6oc7ccccc7c6ccc54)c3)cc(-c3nc(-c4ccccc4)nc(-c4ccccc4)n3)c2)cc1]

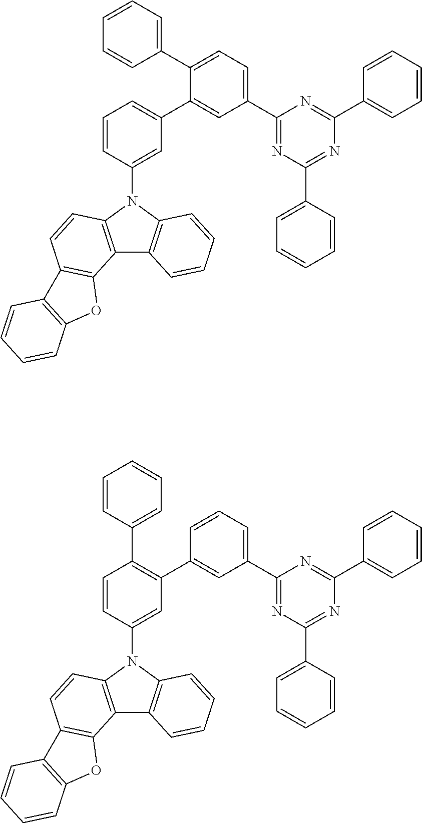 [SMILES: c1ccc(-c2nc(-c3ccccc3)nc(-c3ccc(-c4ccccc4)c(-c4cccc(-n5c6ccccc6c6c7oc8ccccc8c7ccc65)c4)c3)n2)cc1 ; c1ccc(-c2nc(-c3ccccc3)nc(-c3cccc(-c4cc(-n5c6ccccc6c6c7oc8ccccc8c7ccc65)ccc4-c4ccccc4)c3)n2)cc1]

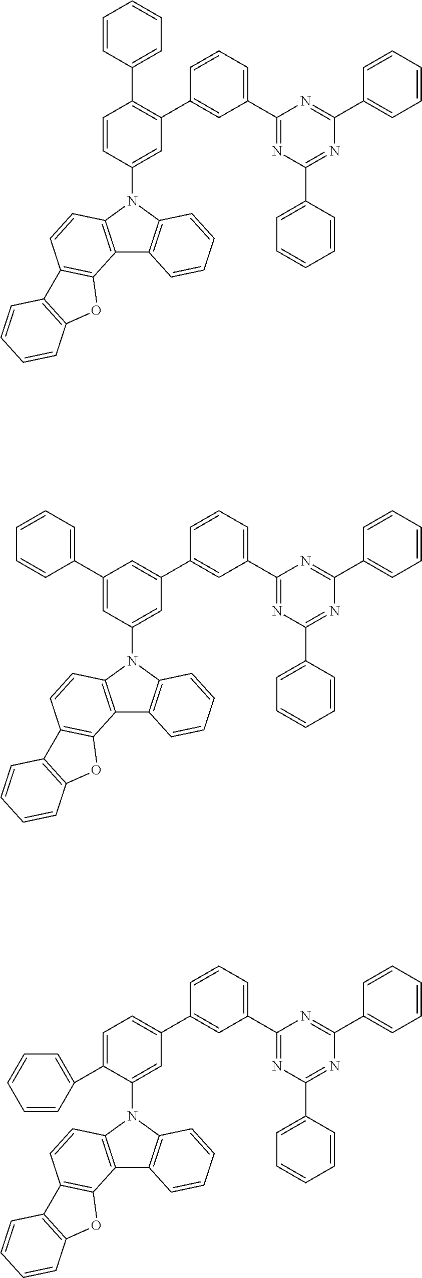 [SMILES: c1ccc(-c2nc(-c3ccccc3)nc(-c3cccc(-c4cc(-n5c6ccccc6c6c7oc8ccccc8c7ccc65)ccc4-c4ccccc4)c3)n2)cc1 ; c1ccc(-c2cc(-c3cccc(-c4nc(-c5ccccc5)nc(-c5ccccc5)n4)c3)cc(-n3c4ccccc4c4c5oc6ccccc6c5ccc43)c2)cc1 ; c1ccc(-c2nc(-c3ccccc3)nc(-c3cccc(-c4ccc(-c5ccccc5)c(-n5c6ccccc6c6c7oc8ccccc8c7ccc65)c4)c3)n2)cc1]

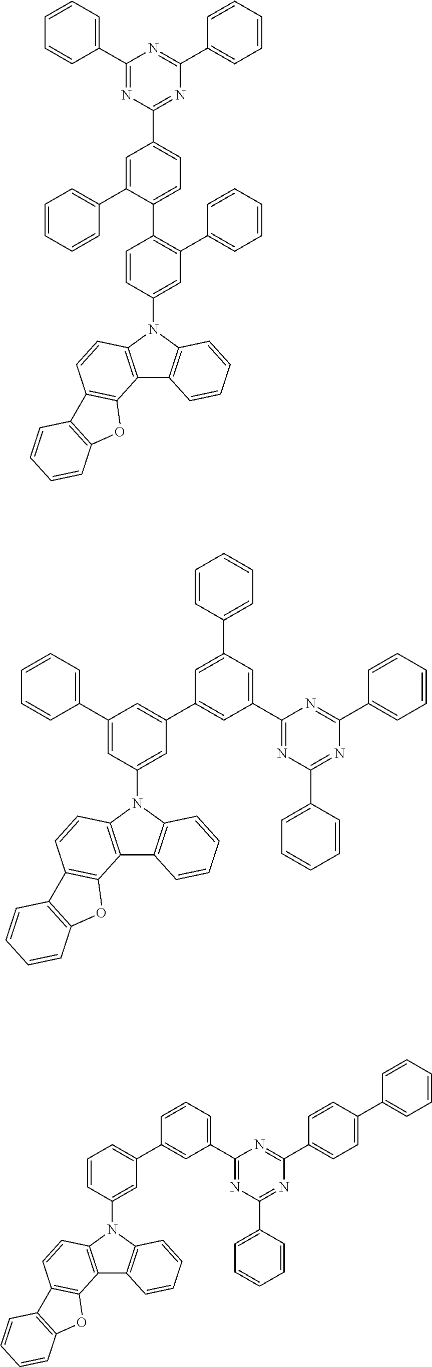 [SMILES: c1ccc(-c2nc(-c3ccccc3)nc(-c3ccc(-c4ccc(-n5c6ccccc6c6c7oc8ccccc8c7ccc65)cc4-c4ccccc4)c(-c4ccccc4)c3)n2)cc1 ; c1ccc(-c2cc(-c3cc(-c4ccccc4)cc(-n4c5ccccc5c5c6oc7ccccc7c6ccc54)c3)cc(-c3nc(-c4ccccc4)nc(-c4ccccc4)n3)c2)cc1 ; c1ccc(-c2ccc(-c3nc(-c4ccccc4)nc(-c4cccc(-c5cccc(-n6c7ccccc7c7c8oc9ccccc9c8ccc76)c5)c4)n3)cc2)cc1]

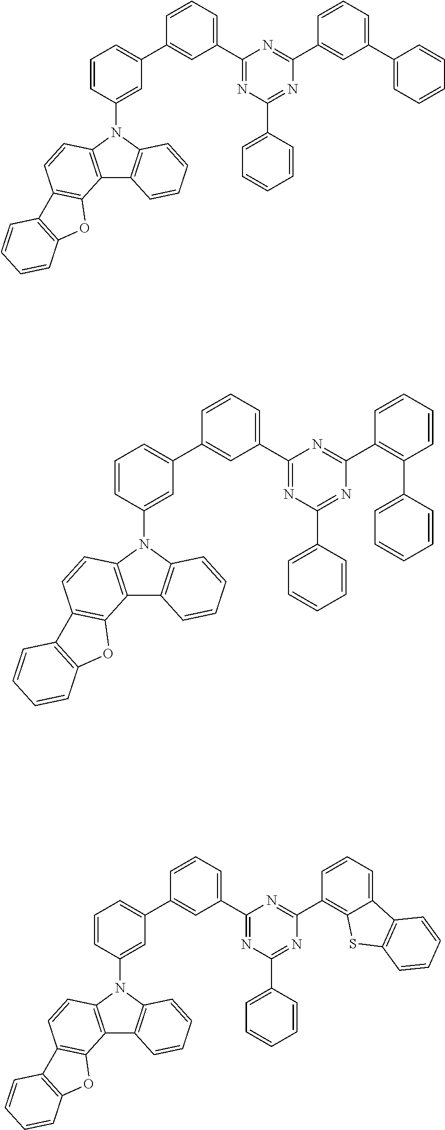 [SMILES: c1ccc(-c2cccc(-c3nc(-c4ccccc4)nc(-c4cccc(-c5cccc(-n6c7ccccc7c7c8oc9ccccc9c8ccc76)c5)c4)n3)c2)cc1 ; c1ccc(-c2nc(-c3cccc(-c4cccc(-n5c6ccccc6c6c7oc8ccccc8c7ccc65)c4)c3)nc(-c3ccccc3-c3ccccc3)n2)cc1 ; c1ccc(-c2nc(-c3cccc(-c4cccc(-n5c6ccccc6c6c7oc8ccccc8c7ccc65)c4)c3)nc(-c3cccc4c3sc3ccccc34)n2)cc1]

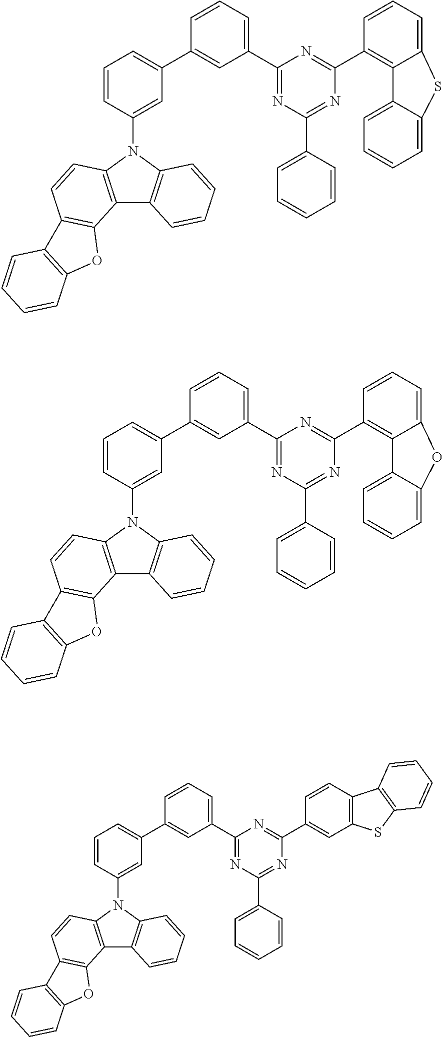 [SMILES: c1ccc(-c2nc(-c3cccc(-c4cccc(-n5c6ccccc6c6c7oc8ccccc8c7ccc65)c4)c3)nc(-c3cccc4sc5ccccc5c34)n2)cc1 ; c1ccc(-c2nc(-c3cccc(-c4cccc(-n5c6ccccc6c6c7oc8ccccc8c7ccc65)c4)c3)nc(-c3cccc4oc5ccccc5c34)n2)cc1 ; c1ccc(-c2nc(-c3cccc(-c4cccc(-n5c6ccccc6c6c7oc8ccccc8c7ccc65)c4)c3)nc(-c3ccc4c(c3)sc3ccccc34)n2)cc1]

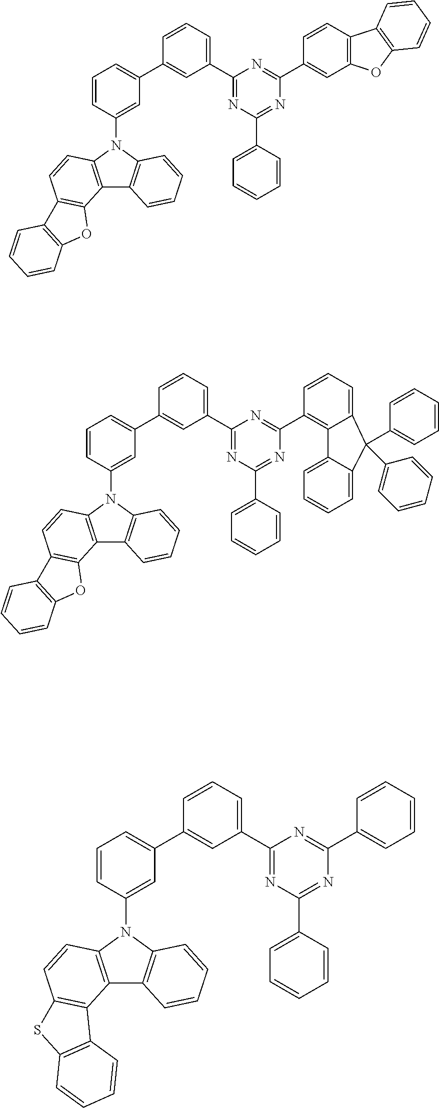 [SMILES: c1ccc(-c2nc(-c3cccc(-c4cccc(-n5c6ccccc6c6c7oc8ccccc8c7ccc65)c4)c3)nc(-c3ccc4c(c3)oc3ccccc34)n2)cc1 ; c1ccc(-c2nc(-c3cccc(-c4cccc(-n5c6ccccc6c6c7oc8ccccc8c7ccc65)c4)c3)nc(-c3cccc4c3-c3ccccc3C4(c3ccccc3)c3ccccc3)n2)cc1 ; c1ccc(-c2nc(-c3ccccc3)nc(-c3cccc(-c4cccc(-n5c6ccccc6c6c7c(ccc65)sc5ccccc57)c4)c3)n2)cc1]

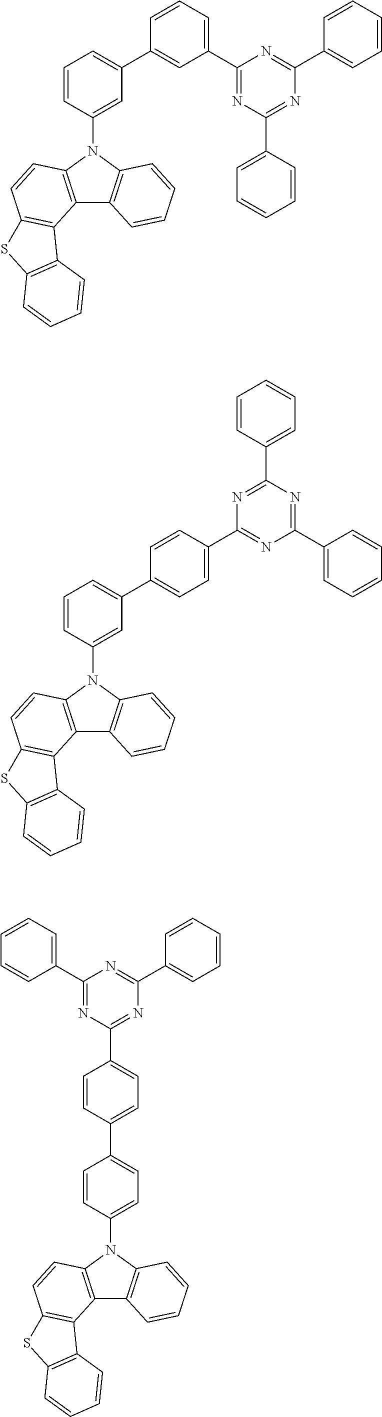 [SMILES: c1ccc(-c2nc(-c3ccccc3)nc(-c3cccc(-c4cccc(-n5c6ccccc6c6c7c(ccc65)sc5ccccc57)c4)c3)n2)cc1 ; c1ccc(-c2nc(-c3ccccc3)nc(-c3ccc(-c4cccc(-n5c6ccccc6c6c7c(ccc65)sc5ccccc57)c4)cc3)n2)cc1 ; c1ccc(-c2nc(-c3ccccc3)nc(-c3ccc(-c4ccc(-n5c6ccccc6c6c7c(ccc65)sc5ccccc57)cc4)cc3)n2)cc1]

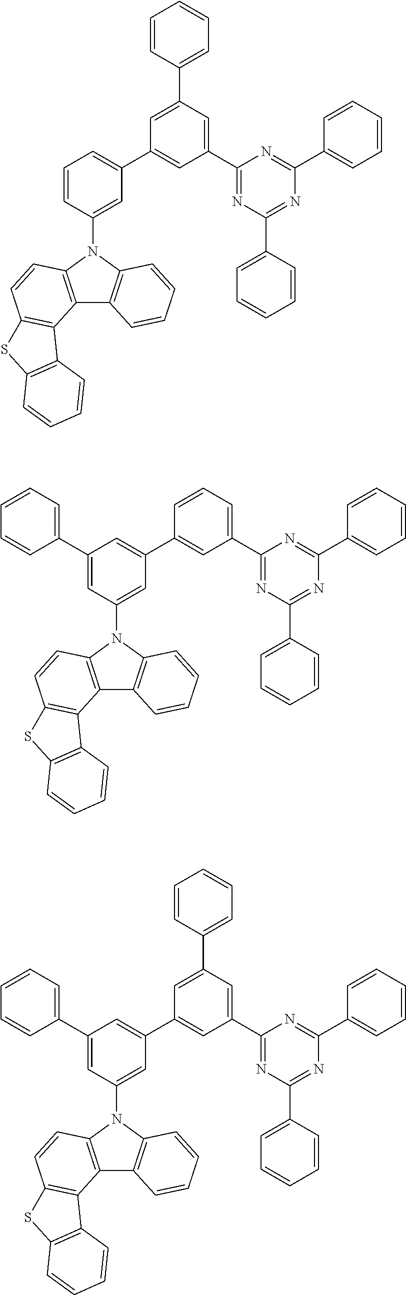 [SMILES: c1ccc(-c2cc(-c3cccc(-n4c5ccccc5c5c6c(ccc54)sc4ccccc46)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4c5c(ccc43)sc3ccccc35)c2)cc1 ; c1ccc(-c2cc(-c3cc(-c4ccccc4)cc(-n4c5ccccc5c5c6c(ccc54)sc4ccccc46)c3)cc(-c3nc(-c4ccccc4)nc(-c4ccccc4)n3)c2)cc1]

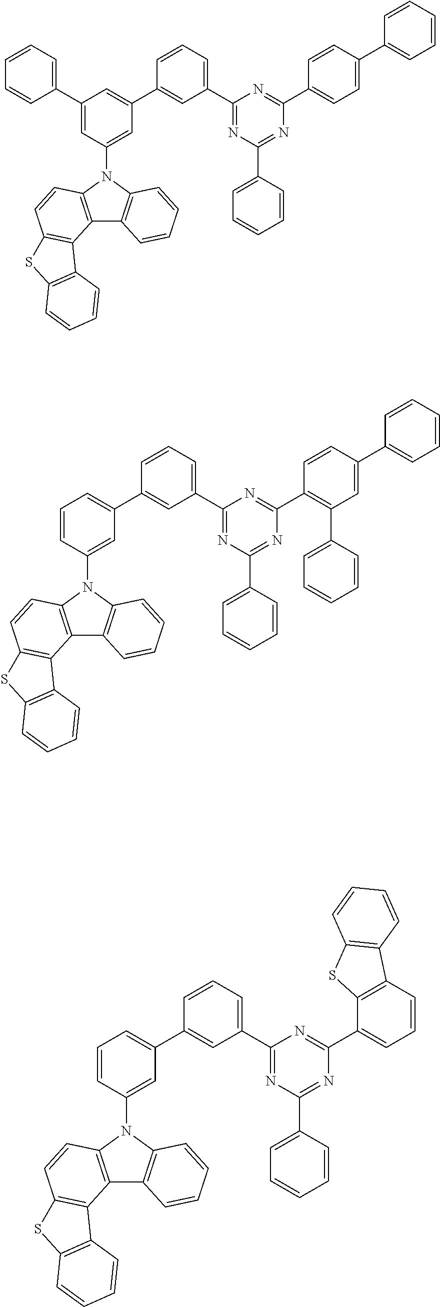 [SMILES: c1ccc(-c2ccc(-c3nc(-c4ccccc4)nc(-c4cccc(-c5cc(-c6ccccc6)cc(-n6c7ccccc7c7c8c(ccc76)sc6ccccc68)c5)c4)n3)cc2)cc1 ; c1ccc(-c2ccc(-c3nc(-c4ccccc4)nc(-c4cccc(-c5cccc(-n6c7ccccc7c7c8c(ccc76)sc6ccccc68)c5)c4)n3)c(-c3ccccc3)c2)cc1 ; c1ccc(-c2nc(-c3cccc(-c4cccc(-n5c6ccccc6c6c7c(ccc65)sc5ccccc57)c4)c3)nc(-c3cccc4c3sc3ccccc34)n2)cc1]

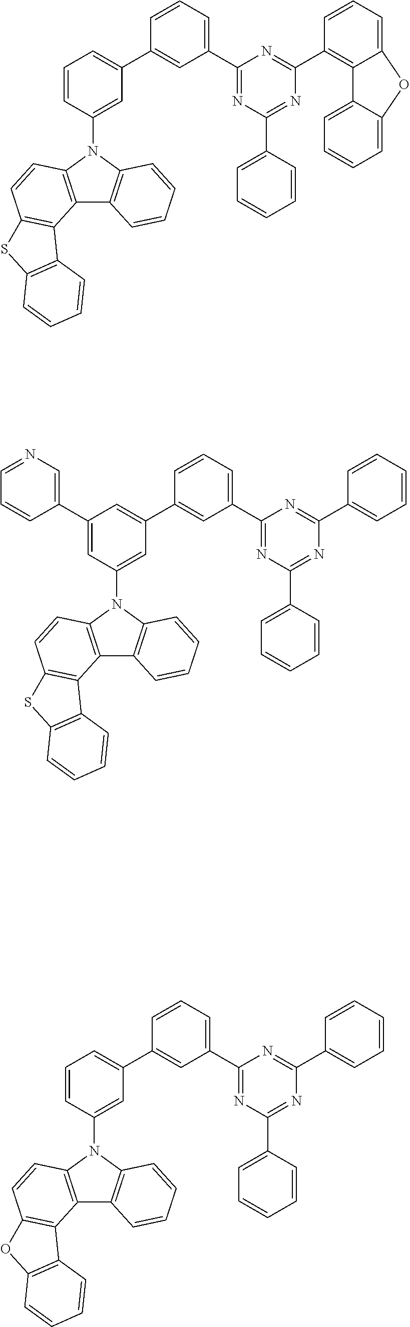 [SMILES: c1ccc(-c2nc(-c3cccc(-c4cccc(-n5c6ccccc6c6c7c(ccc65)sc5ccccc57)c4)c3)nc(-c3cccc4oc5ccccc5c34)n2)cc1 ; c1ccc(-c2nc(-c3ccccc3)nc(-c3cccc(-c4cc(-c5cccnc5)cc(-n5c6ccccc6c6c7c(ccc65)sc5ccccc57)c4)c3)n2)cc1 ; c1ccc(-c2nc(-c3ccccc3)nc(-c3cccc(-c4cccc(-n5c6ccccc6c6c7c(ccc65)oc5ccccc57)c4)c3)n2)cc1]

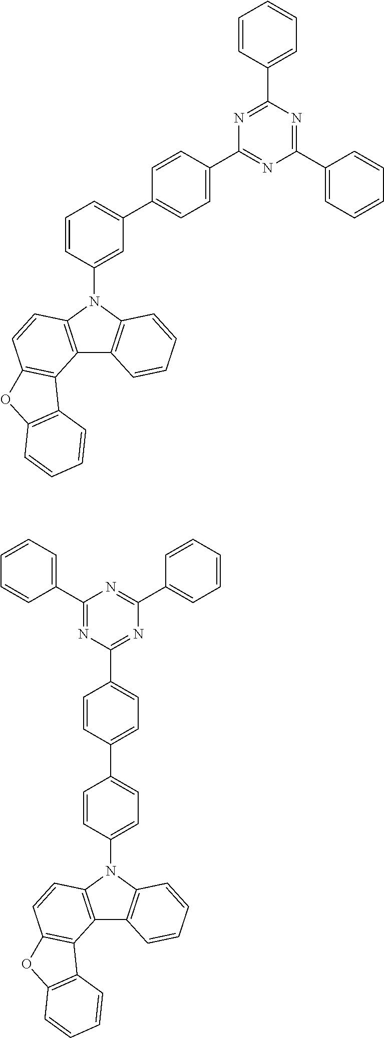 [SMILES: c1ccc(-c2nc(-c3ccccc3)nc(-c3ccc(-c4cccc(-n5c6ccccc6c6c7c(ccc65)oc5ccccc57)c4)cc3)n2)cc1 ; c1ccc(-c2nc(-c3ccccc3)nc(-c3ccc(-c4ccc(-n5c6ccccc6c6c7c(ccc65)oc5ccccc57)cc4)cc3)n2)cc1]

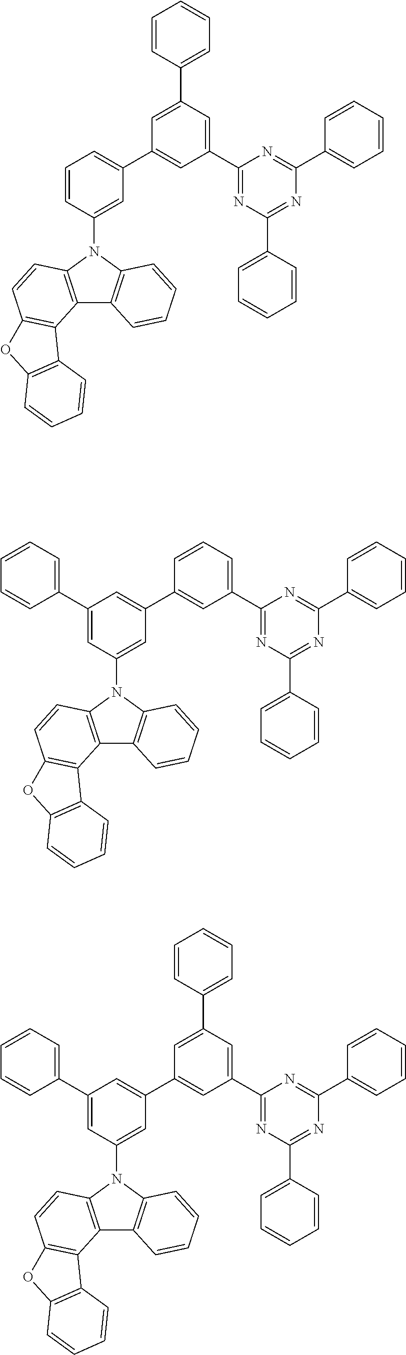 [SMILES: c1ccc(-c2cc(-c3cccc(-n4c5ccccc5c5c6c(ccc54)oc4ccccc46)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4c5c(ccc43)oc3ccccc35)c2)cc1 ; c1ccc(-c2cc(-c3cc(-c4ccccc4)cc(-n4c5ccccc5c5c6c(ccc54)oc4ccccc46)c3)cc(-c3nc(-c4ccccc4)nc(-c4ccccc4)n3)c2)cc1]

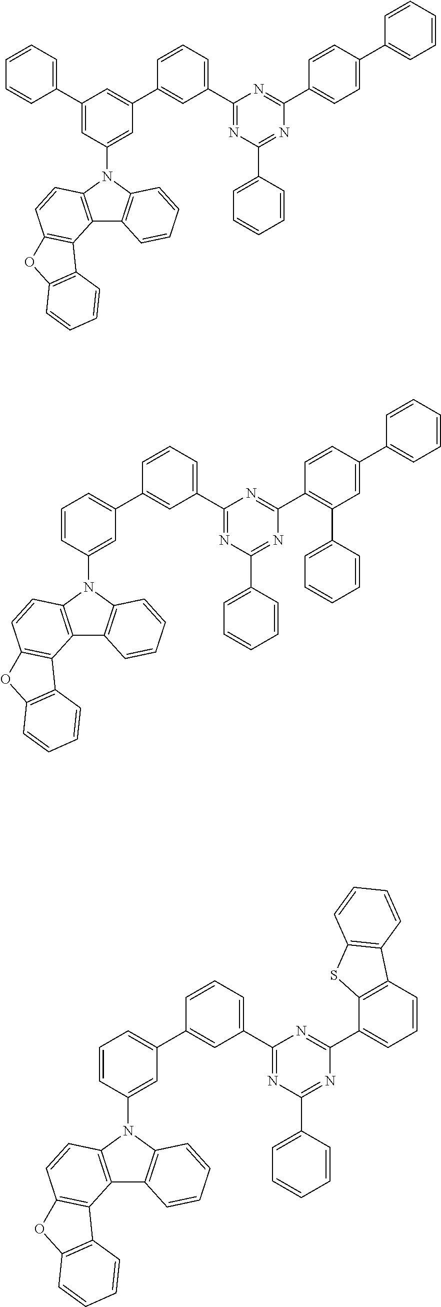 [SMILES: c1ccc(-c2ccc(-c3nc(-c4ccccc4)nc(-c4cccc(-c5cc(-c6ccccc6)cc(-n6c7ccccc7c7c8c(ccc76)oc6ccccc68)c5)c4)n3)cc2)cc1 ; c1ccc(-c2ccc(-c3nc(-c4ccccc4)nc(-c4cccc(-c5cccc(-n6c7ccccc7c7c8c(ccc76)oc6ccccc68)c5)c4)n3)c(-c3ccccc3)c2)cc1 ; c1ccc(-c2nc(-c3cccc(-c4cccc(-n5c6ccccc6c6c7c(ccc65)oc5ccccc57)c4)c3)nc(-c3cccc4c3sc3ccccc34)n2)cc1]

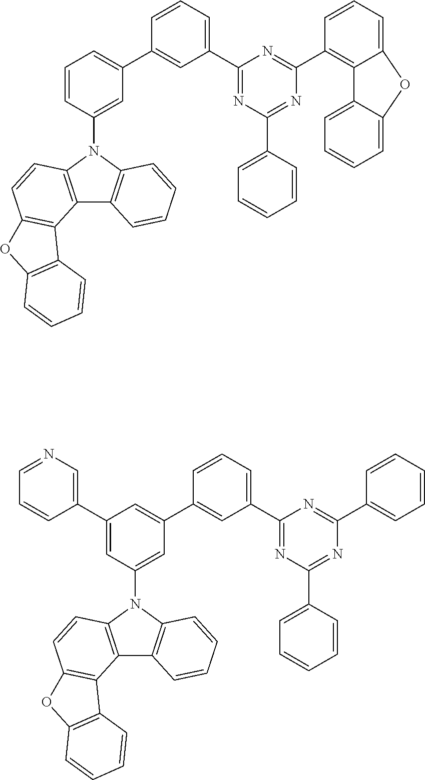 [SMILES: c1ccc(-c2nc(-c3cccc(-c4cccc(-n5c6ccccc6c6c7c(ccc65)oc5ccccc57)c4)c3)nc(-c3cccc4oc5ccccc5c34)n2)cc1 ; c1ccc(-c2nc(-c3ccccc3)nc(-c3cccc(-c4cc(-c5cccnc5)cc(-n5c6ccccc6c6c7c(ccc65)oc5ccccc57)c4)c3)n2)cc1]

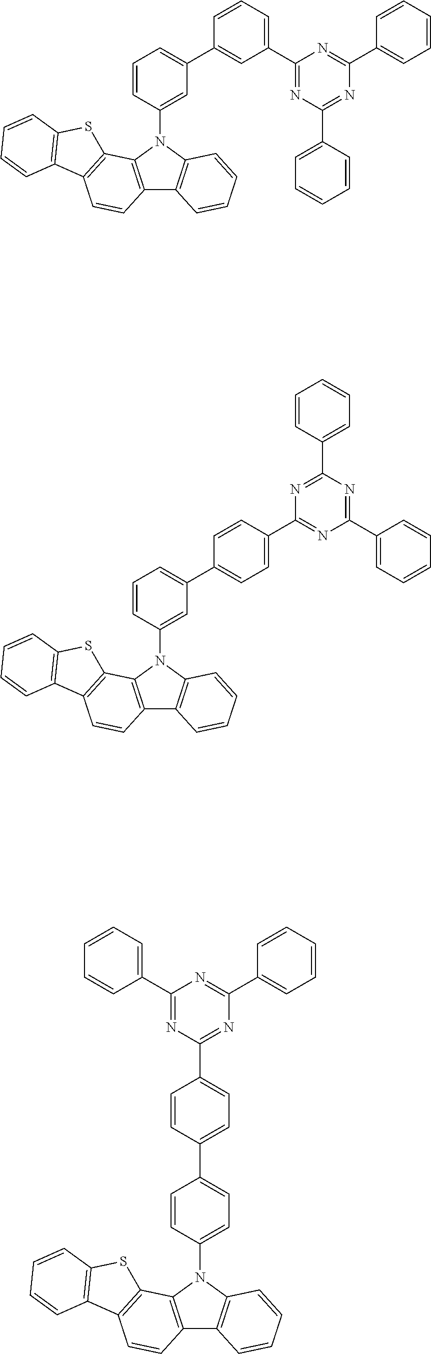 [SMILES: c1ccc(-c2nc(-c3ccccc3)nc(-c3cccc(-c4cccc(-n5c6ccccc6c6ccc7c8ccccc8sc7c65)c4)c3)n2)cc1 ; c1ccc(-c2nc(-c3ccccc3)nc(-c3ccc(-c4cccc(-n5c6ccccc6c6ccc7c8ccccc8sc7c65)c4)cc3)n2)cc1 ; c1ccc(-c2nc(-c3ccccc3)nc(-c3ccc(-c4ccc(-n5c6ccccc6c6ccc7c8ccccc8sc7c65)cc4)cc3)n2)cc1]

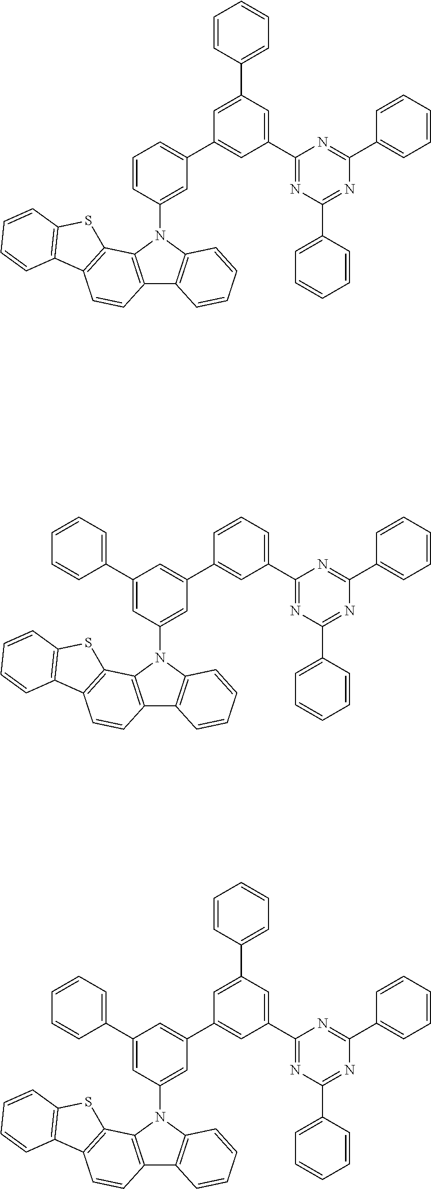 [SMILES: c1ccc(-c2cc(-c3cccc(-n4c5ccccc5c5ccc6c7ccccc7sc6c54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4ccc5c6ccccc6sc5c43)c2)cc1 ; c1ccc(-c2cc(-c3cc(-c4ccccc4)cc(-n4c5ccccc5c5ccc6c7ccccc7sc6c54)c3)cc(-c3nc(-c4ccccc4)nc(-c4ccccc4)n3)c2)cc1]

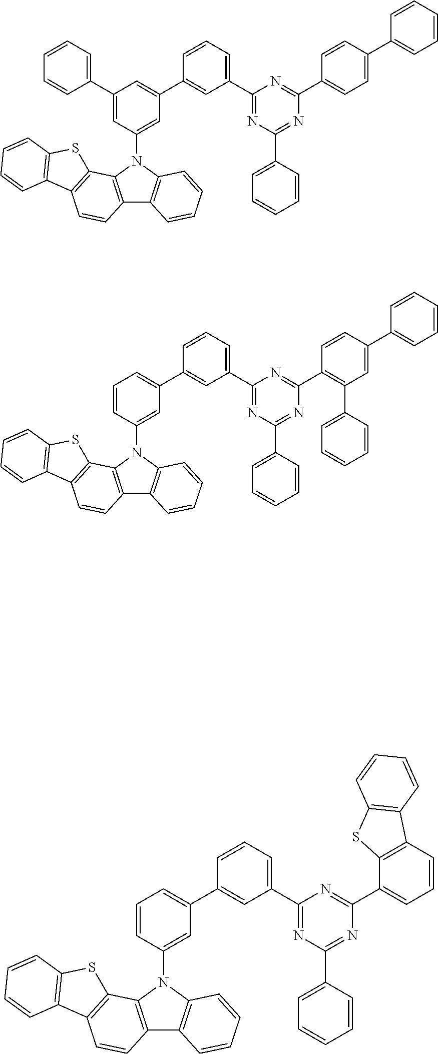 [SMILES: c1ccc(-c2ccc(-c3nc(-c4ccccc4)nc(-c4cccc(-c5cc(-c6ccccc6)cc(-n6c7ccccc7c7ccc8c9ccccc9sc8c76)c5)c4)n3)cc2)cc1 ; c1ccc(-c2ccc(-c3nc(-c4ccccc4)nc(-c4cccc(-c5cccc(-n6c7ccccc7c7ccc8c9ccccc9sc8c76)c5)c4)n3)c(-c3ccccc3)c2)cc1 ; c1ccc(-c2nc(-c3cccc(-c4cccc(-n5c6ccccc6c6ccc7c8ccccc8sc7c65)c4)c3)nc(-c3cccc4c3sc3ccccc34)n2)cc1]

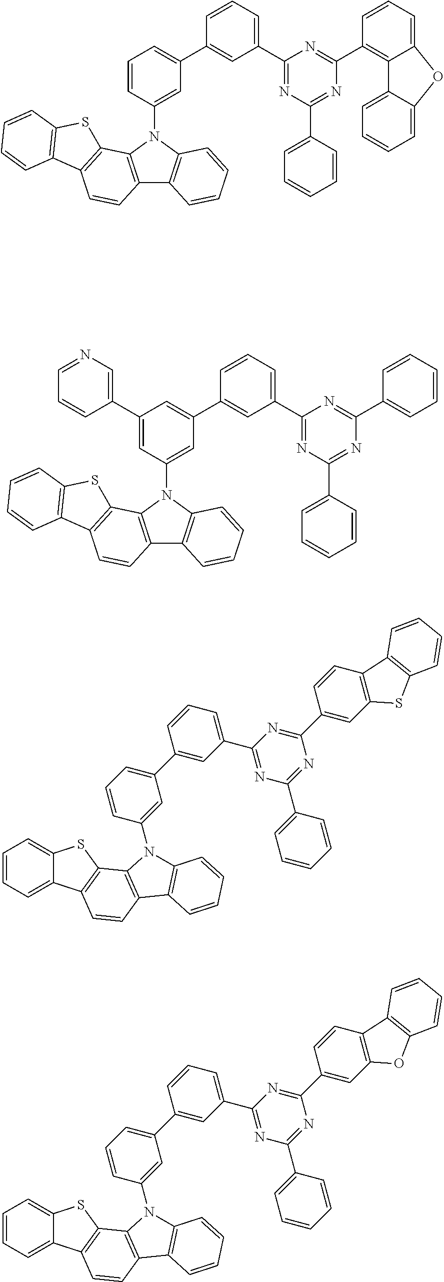 [SMILES: c1ccc(-c2nc(-c3cccc(-c4cccc(-n5c6ccccc6c6ccc7c8ccccc8sc7c65)c4)c3)nc(-c3cccc4oc5ccccc5c34)n2)cc1 ; c1ccc(-c2nc(-c3ccccc3)nc(-c3cccc(-c4cc(-c5cccnc5)cc(-n5c6ccccc6c6ccc7c8ccccc8sc7c65)c4)c3)n2)cc1 ; c1ccc(-c2nc(-c3cccc(-c4cccc(-n5c6ccccc6c6ccc7c8ccccc8sc7c65)c4)c3)nc(-c3ccc4c(c3)sc3ccccc34)n2)cc1 ; c1ccc(-c2nc(-c3cccc(-c4cccc(-n5c6ccccc6c6ccc7c8ccccc8sc7c65)c4)c3)nc(-c3ccc4c(c3)oc3ccccc34)n2)cc1]

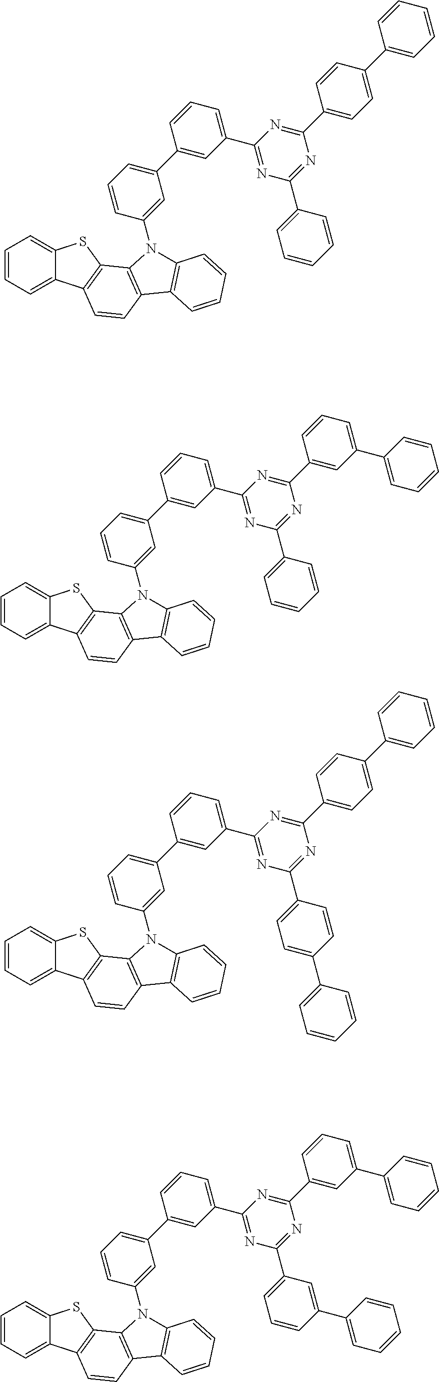 [SMILES: c1ccc(-c2ccc(-c3nc(-c4ccccc4)nc(-c4cccc(-c5cccc(-n6c7ccccc7c7ccc8c9ccccc9sc8c76)c5)c4)n3)cc2)cc1 ; c1ccc(-c2cccc(-c3nc(-c4ccccc4)nc(-c4cccc(-c5cccc(-n6c7ccccc7c7ccc8c9ccccc9sc8c76)c5)c4)n3)c2)cc1 ; c1ccc(-c2ccc(-c3nc(-c4ccc(-c5ccccc5)cc4)nc(-c4cccc(-c5cccc(-n6c7ccccc7c7ccc8c9ccccc9sc8c76)c5)c4)n3)cc2)cc1 ; c1ccc(-c2cccc(-c3nc(-c4cccc(-c5ccccc5)c4)nc(-c4cccc(-c5cccc(-n6c7ccccc7c7ccc8c9ccccc9sc8c76)c5)c4)n3)c2)cc1]

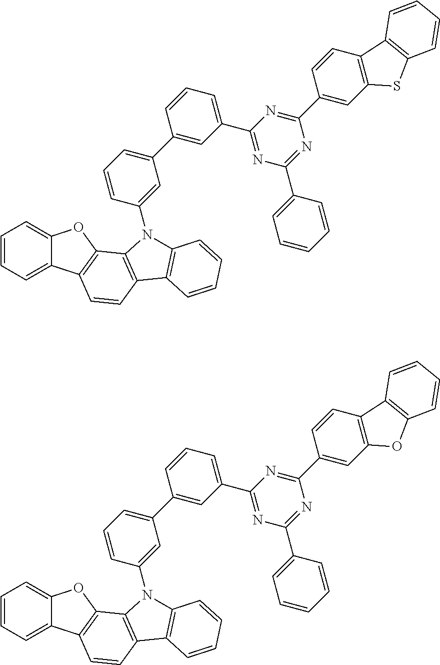 [SMILES: c1ccc(-c2nc(-c3cccc(-c4cccc(-n5c6ccccc6c6ccc7c8ccccc8oc7c65)c4)c3)nc(-c3ccc4c(c3)sc3ccccc34)n2)cc1 ; c1ccc(-c2nc(-c3cccc(-c4cccc(-n5c6ccccc6c6ccc7c8ccccc8oc7c65)c4)c3)nc(-c3ccc4c(c3)oc3ccccc34)n2)cc1]

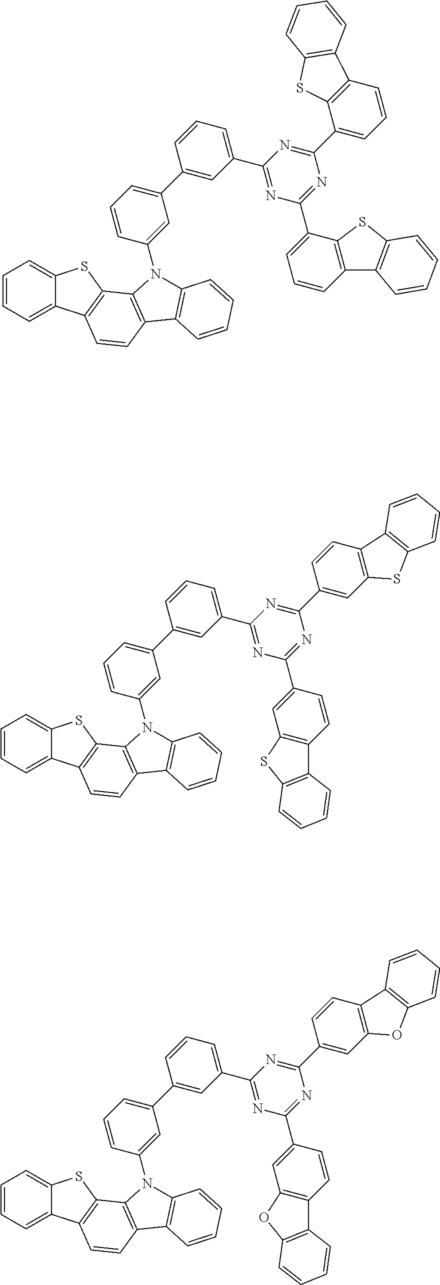 [SMILES: c1cc(-c2cccc(-n3c4ccccc4c4ccc5c6ccccc6sc5c43)c2)cc(-c2nc(-c3cccc4c3sc3ccccc34)nc(-c3cccc4c3sc3ccccc34)n2)c1 ; c1cc(-c2cccc(-n3c4ccccc4c4ccc5c6ccccc6sc5c43)c2)cc(-c2nc(-c3ccc4c(c3)sc3ccccc34)nc(-c3ccc4c(c3)sc3ccccc34)n2)c1 ; c1cc(-c2cccc(-n3c4ccccc4c4ccc5c6ccccc6sc5c43)c2)cc(-c2nc(-c3ccc4c(c3)oc3ccccc34)nc(-c3ccc4c(c3)oc3ccccc34)n2)c1]

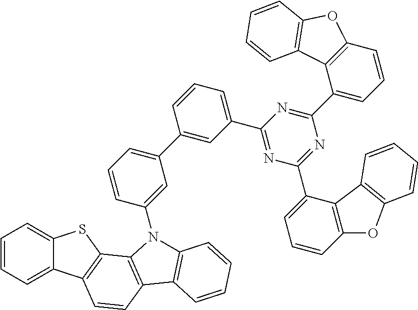 [SMILES: c1cc(-c2cccc(-n3c4ccccc4c4ccc5c6ccccc6sc5c43)c2)cc(-c2nc(-c3cccc4oc5ccccc5c34)nc(-c3cccc4oc5ccccc5c34)n2)c1]

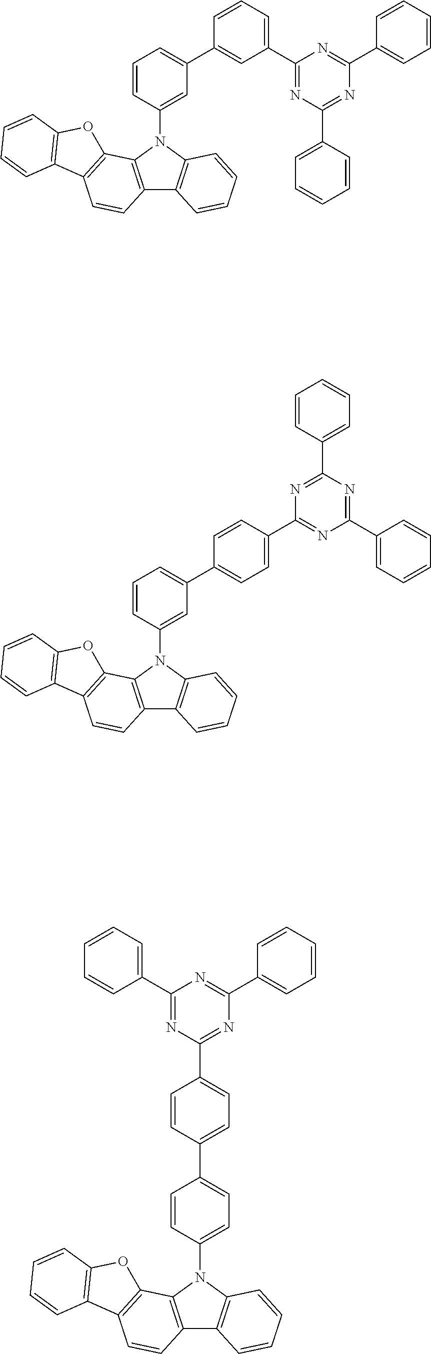 [SMILES: c1ccc(-c2nc(-c3ccccc3)nc(-c3cccc(-c4cccc(-n5c6ccccc6c6ccc7c8ccccc8oc7c65)c4)c3)n2)cc1 ; c1ccc(-c2nc(-c3ccccc3)nc(-c3ccc(-c4cccc(-n5c6ccccc6c6ccc7c8ccccc8oc7c65)c4)cc3)n2)cc1 ; c1ccc(-c2nc(-c3ccccc3)nc(-c3ccc(-c4ccc(-n5c6ccccc6c6ccc7c8ccccc8oc7c65)cc4)cc3)n2)cc1]

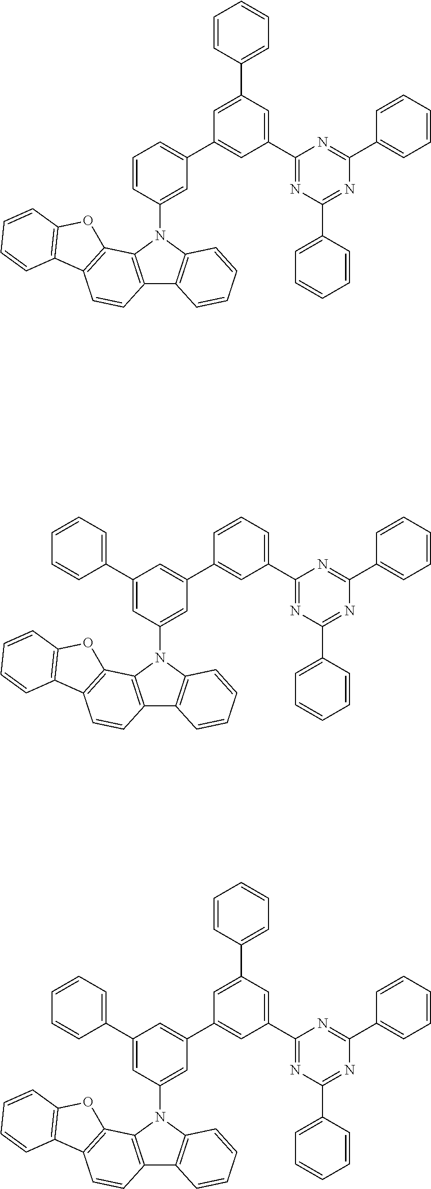 [SMILES: c1ccc(-c2cc(-c3cccc(-n4c5ccccc5c5ccc6c7ccccc7oc6c54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4ccc5c6ccccc6oc5c43)c2)cc1 ; c1ccc(-c2cc(-c3cc(-c4ccccc4)cc(-n4c5ccccc5c5ccc6c7ccccc7oc6c54)c3)cc(-c3nc(-c4ccccc4)nc(-c4ccccc4)n3)c2)cc1]

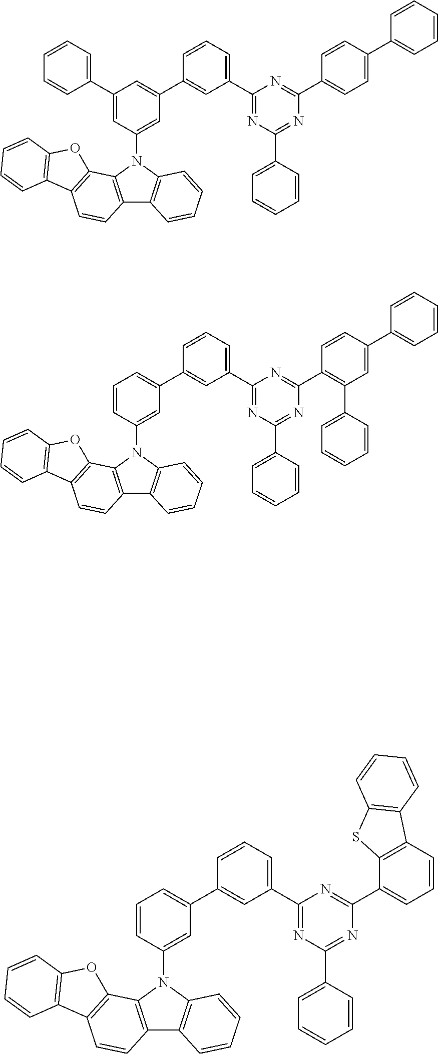 [SMILES: c1ccc(-c2ccc(-c3nc(-c4ccccc4)nc(-c4cccc(-c5cc(-c6ccccc6)cc(-n6c7ccccc7c7ccc8c9ccccc9oc8c76)c5)c4)n3)cc2)cc1 ; c1ccc(-c2ccc(-c3nc(-c4ccccc4)nc(-c4cccc(-c5cccc(-n6c7ccccc7c7ccc8c9ccccc9oc8c76)c5)c4)n3)c(-c3ccccc3)c2)cc1 ; c1ccc(-c2nc(-c3cccc(-c4cccc(-n5c6ccccc6c6ccc7c8ccccc8oc7c65)c4)c3)nc(-c3cccc4c3sc3ccccc34)n2)cc1]

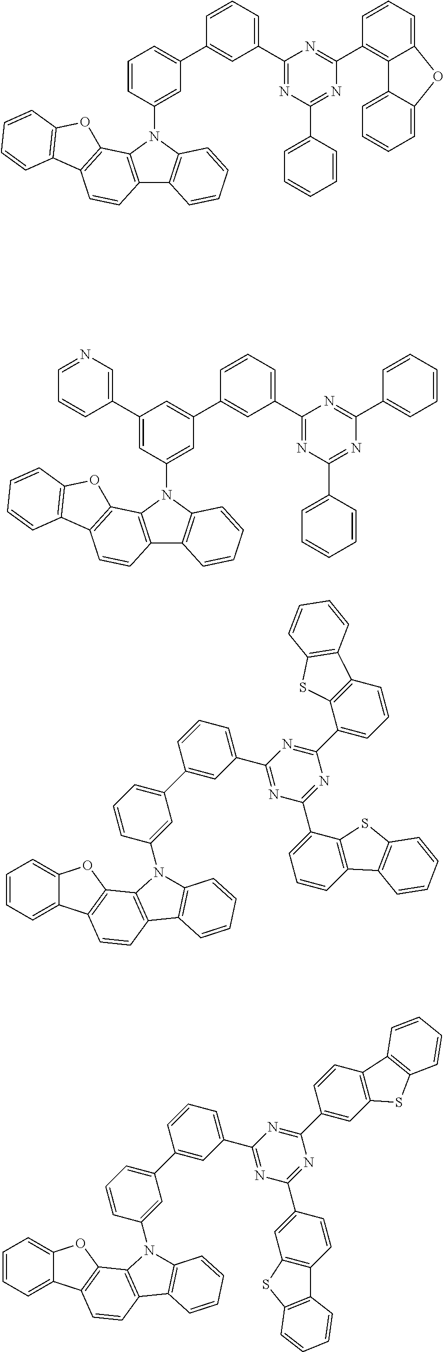 [SMILES: c1ccc(-c2nc(-c3cccc(-c4cccc(-n5c6ccccc6c6ccc7c8ccccc8oc7c65)c4)c3)nc(-c3cccc4oc5ccccc5c34)n2)cc1 ; c1ccc(-c2nc(-c3ccccc3)nc(-c3cccc(-c4cc(-c5cccnc5)cc(-n5c6ccccc6c6ccc7c8ccccc8oc7c65)c4)c3)n2)cc1 ; c1cc(-c2cccc(-n3c4ccccc4c4ccc5c6ccccc6oc5c43)c2)cc(-c2nc(-c3cccc4c3sc3ccccc34)nc(-c3cccc4c3sc3ccccc34)n2)c1 ; c1cc(-c2cccc(-n3c4ccccc4c4ccc5c6ccccc6oc5c43)c2)cc(-c2nc(-c3ccc4c(c3)sc3ccccc34)nc(-c3ccc4c(c3)sc3ccccc34)n2)c1]

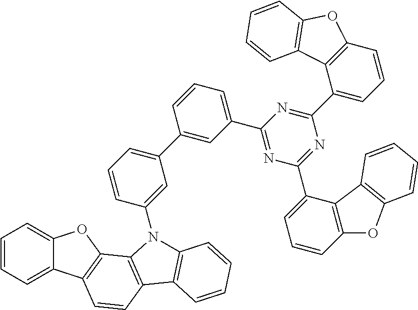 [SMILES: c1cc(-c2cccc(-n3c4ccccc4c4ccc5c6ccccc6oc5c43)c2)cc(-c2nc(-c3cccc4oc5ccccc5c34)nc(-c3cccc4oc5ccccc5c34)n2)c1]

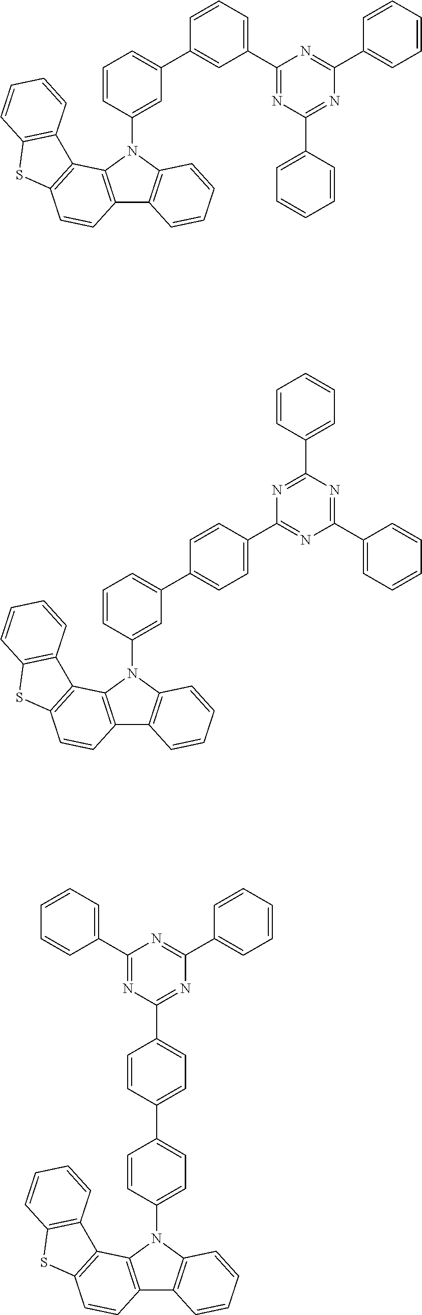 [SMILES: c1ccc(-c2nc(-c3ccccc3)nc(-c3cccc(-c4cccc(-n5c6ccccc6c6ccc7sc8ccccc8c7c65)c4)c3)n2)cc1 ; c1ccc(-c2nc(-c3ccccc3)nc(-c3ccc(-c4cccc(-n5c6ccccc6c6ccc7sc8ccccc8c7c65)c4)cc3)n2)cc1 ; c1ccc(-c2nc(-c3ccccc3)nc(-c3ccc(-c4ccc(-n5c6ccccc6c6ccc7sc8ccccc8c7c65)cc4)cc3)n2)cc1]

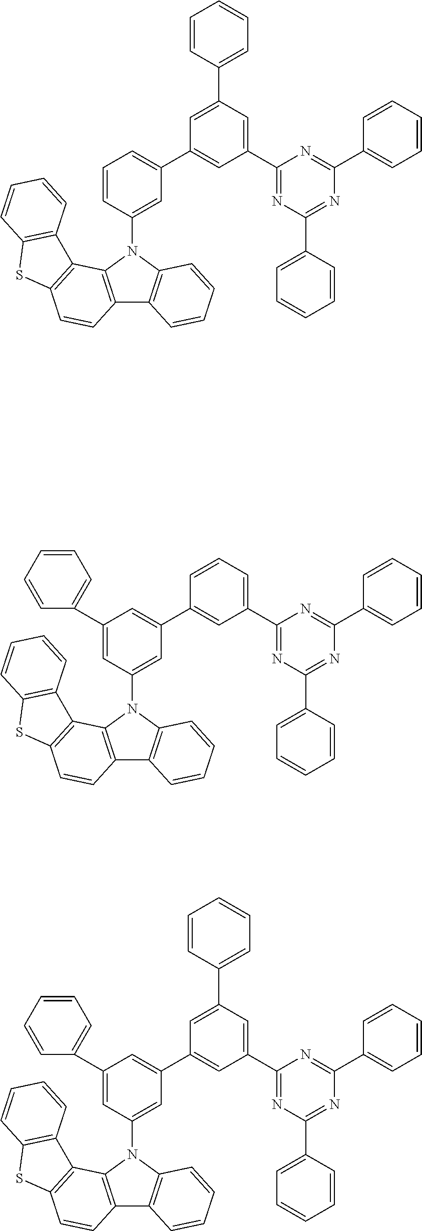 [SMILES: c1ccc(-c2cc(-c3cccc(-n4c5ccccc5c5ccc6sc7ccccc7c6c54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4ccc5sc6ccccc6c5c43)c2)cc1 ; c1ccc(-c2cc(-c3cc(-c4ccccc4)cc(-n4c5ccccc5c5ccc6sc7ccccc7c6c54)c3)cc(-c3nc(-c4ccccc4)nc(-c4ccccc4)n3)c2)cc1]

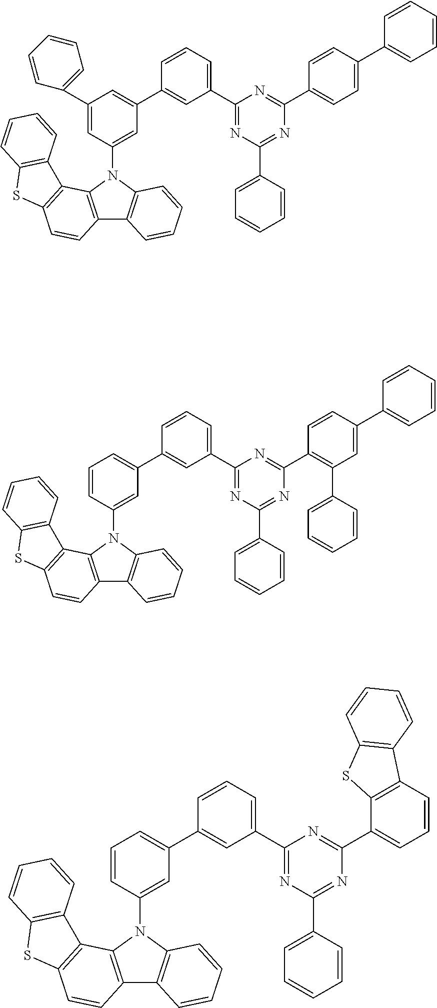 [SMILES: c1ccc(-c2ccc(-c3nc(-c4ccccc4)nc(-c4cccc(-c5cc(-c6ccccc6)cc(-n6c7ccccc7c7ccc8sc9ccccc9c8c76)c5)c4)n3)cc2)cc1 ; c1ccc(-c2ccc(-c3nc(-c4ccccc4)nc(-c4cccc(-c5cccc(-n6c7ccccc7c7ccc8sc9ccccc9c8c76)c5)c4)n3)c(-c3ccccc3)c2)cc1 ; c1ccc(-c2nc(-c3cccc(-c4cccc(-n5c6ccccc6c6ccc7sc8ccccc8c7c65)c4)c3)nc(-c3cccc4c3sc3ccccc34)n2)cc1]

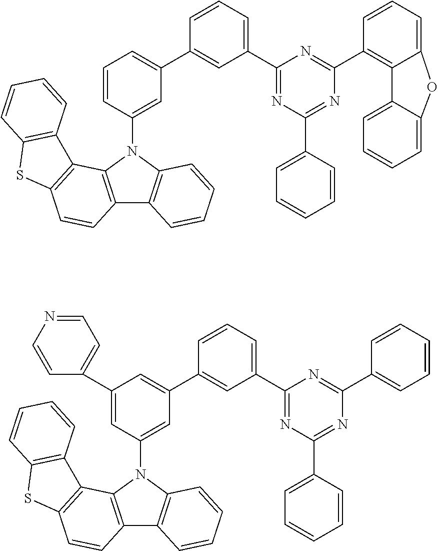 [SMILES: c1ccc(-c2nc(-c3cccc(-c4cccc(-n5c6ccccc6c6ccc7sc8ccccc8c7c65)c4)c3)nc(-c3cccc4oc5ccccc5c34)n2)cc1 ; c1ccc(-c2nc(-c3ccccc3)nc(-c3cccc(-c4cc(-c5ccncc5)cc(-n5c6ccccc6c6ccc7sc8ccccc8c7c65)c4)c3)n2)cc1]

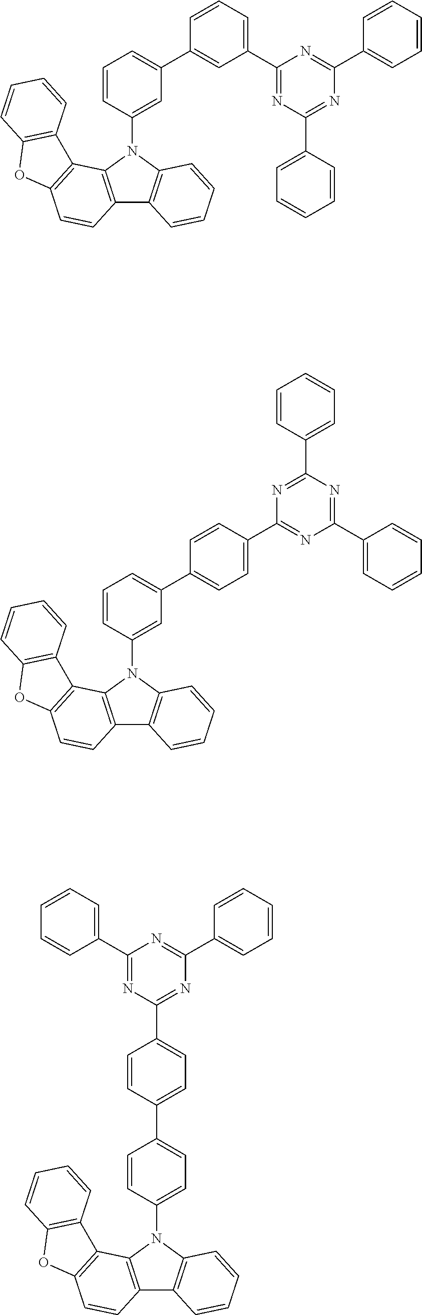 [SMILES: c1ccc(-c2nc(-c3ccccc3)nc(-c3cccc(-c4cccc(-n5c6ccccc6c6ccc7oc8ccccc8c7c65)c4)c3)n2)cc1 ; c1ccc(-c2nc(-c3ccccc3)nc(-c3ccc(-c4cccc(-n5c6ccccc6c6ccc7oc8ccccc8c7c65)c4)cc3)n2)cc1 ; c1ccc(-c2nc(-c3ccccc3)nc(-c3ccc(-c4ccc(-n5c6ccccc6c6ccc7oc8ccccc8c7c65)cc4)cc3)n2)cc1]

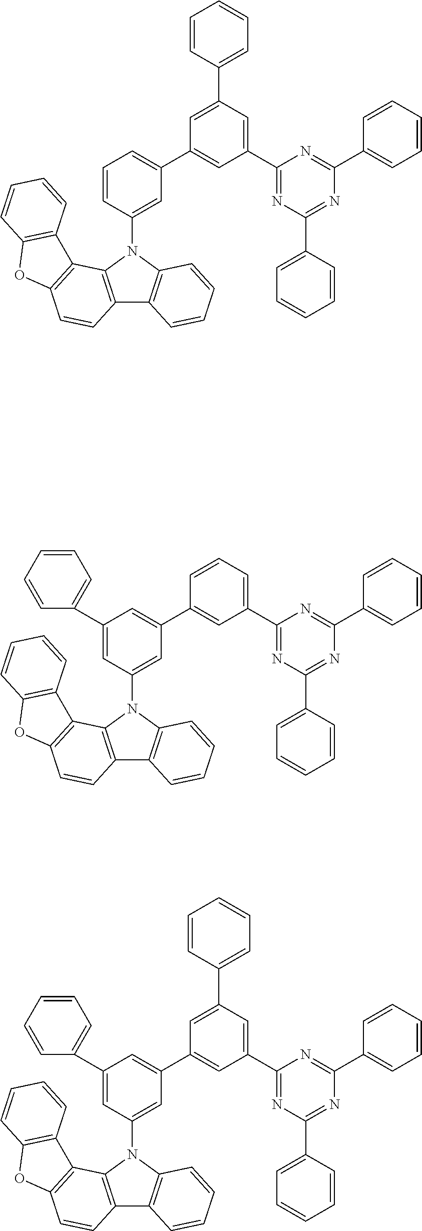 [SMILES: c1ccc(-c2cc(-c3cccc(-n4c5ccccc5c5ccc6oc7ccccc7c6c54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4ccc5oc6ccccc6c5c43)c2)cc1 ; c1ccc(-c2cc(-c3cc(-c4ccccc4)cc(-n4c5ccccc5c5ccc6oc7ccccc7c6c54)c3)cc(-c3nc(-c4ccccc4)nc(-c4ccccc4)n3)c2)cc1]

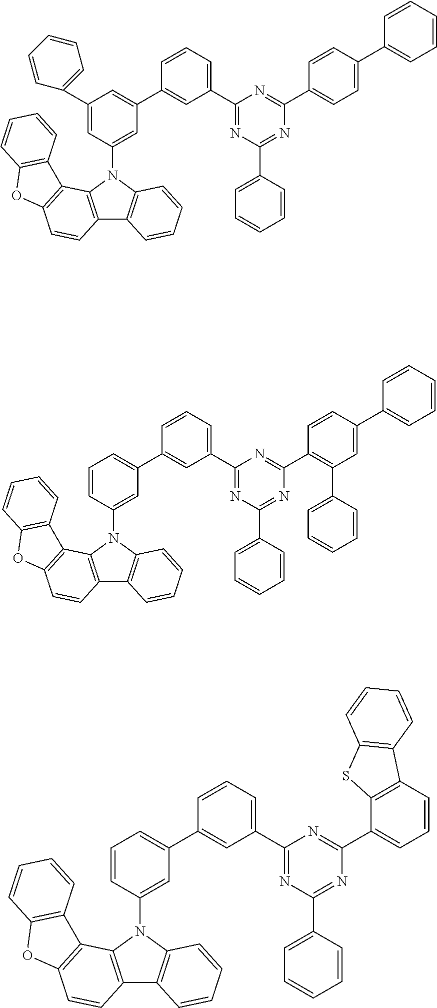 [SMILES: c1ccc(-c2ccc(-c3nc(-c4ccccc4)nc(-c4cccc(-c5cc(-c6ccccc6)cc(-n6c7ccccc7c7ccc8oc9ccccc9c8c76)c5)c4)n3)cc2)cc1 ; c1ccc(-c2ccc(-c3nc(-c4ccccc4)nc(-c4cccc(-c5cccc(-n6c7ccccc7c7ccc8oc9ccccc9c8c76)c5)c4)n3)c(-c3ccccc3)c2)cc1 ; c1ccc(-c2nc(-c3cccc(-c4cccc(-n5c6ccccc6c6ccc7oc8ccccc8c7c65)c4)c3)nc(-c3cccc4c3sc3ccccc34)n2)cc1]

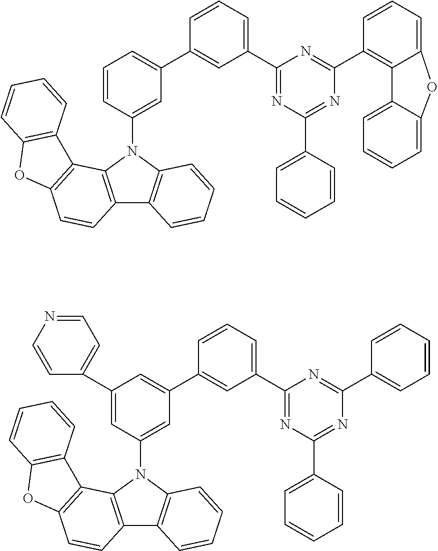 [SMILES: c1ccc(-c2nc(-c3cccc(-c4cccc(-n5c6ccccc6c6ccc7oc8ccccc8c7c65)c4)c3)nc(-c3cccc4oc5ccccc5c34)n2)cc1 ; c1ccc(-c2nc(-c3ccccc3)nc(-c3cccc(-c4cc(-c5ccncc5)cc(-n5c6ccccc6c6ccc7oc8ccccc8c7c65)c4)c3)n2)cc1]

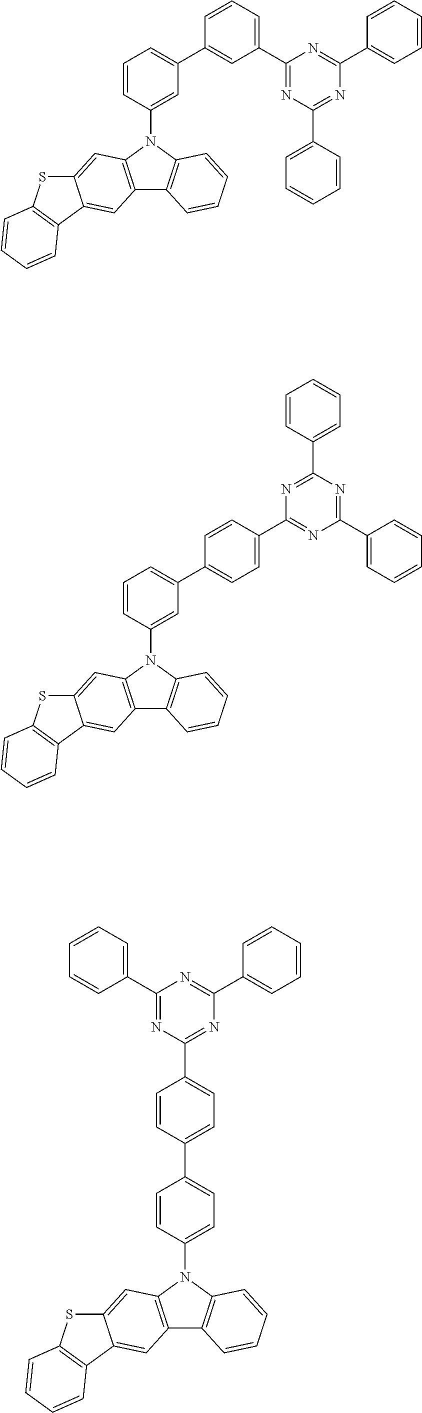 [SMILES: c1ccc(-c2nc(-c3ccccc3)nc(-c3cccc(-c4cccc(-n5c6ccccc6c6cc7c(cc65)sc5ccccc57)c4)c3)n2)cc1 ; c1ccc(-c2nc(-c3ccccc3)nc(-c3ccc(-c4cccc(-n5c6ccccc6c6cc7c(cc65)sc5ccccc57)c4)cc3)n2)cc1 ; c1ccc(-c2nc(-c3ccccc3)nc(-c3ccc(-c4ccc(-n5c6ccccc6c6cc7c(cc65)sc5ccccc57)cc4)cc3)n2)cc1]

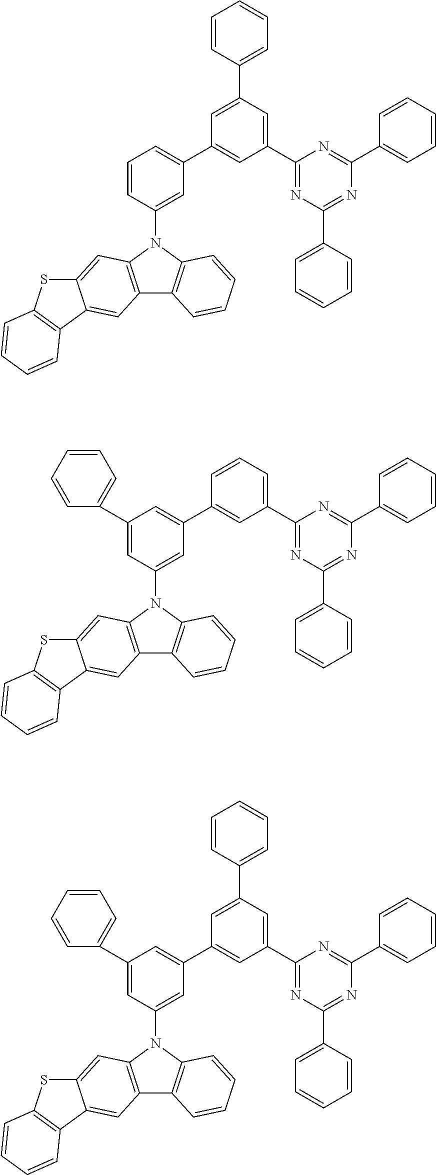 [SMILES: c1ccc(-c2cc(-c3cccc(-n4c5ccccc5c5cc6c(cc54)sc4ccccc46)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4cc5c(cc43)sc3ccccc35)c2)cc1 ; c1ccc(-c2cc(-c3cc(-c4ccccc4)cc(-n4c5ccccc5c5cc6c(cc54)sc4ccccc46)c3)cc(-c3nc(-c4ccccc4)nc(-c4ccccc4)n3)c2)cc1]

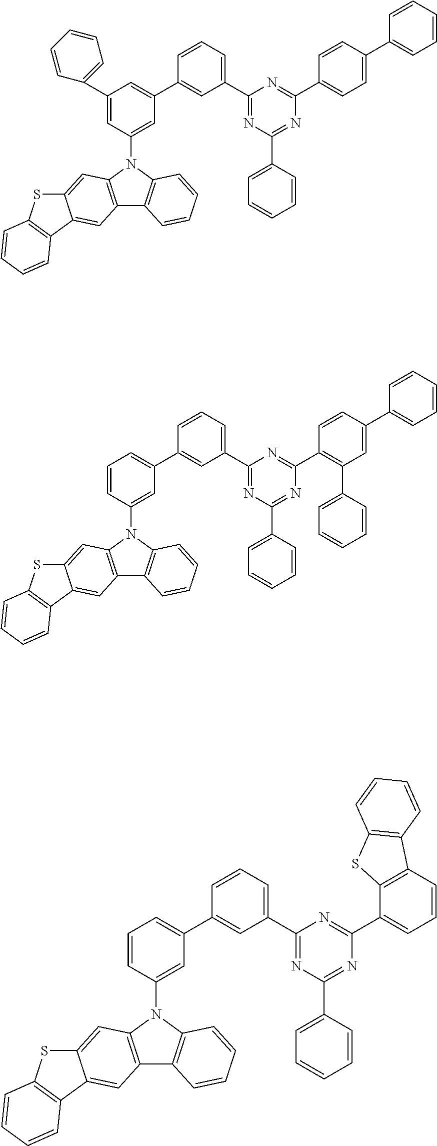 [SMILES: c1ccc(-c2ccc(-c3nc(-c4ccccc4)nc(-c4cccc(-c5cc(-c6ccccc6)cc(-n6c7ccccc7c7cc8c(cc76)sc6ccccc68)c5)c4)n3)cc2)cc1 ; c1ccc(-c2ccc(-c3nc(-c4ccccc4)nc(-c4cccc(-c5cccc(-n6c7ccccc7c7cc8c(cc76)sc6ccccc68)c5)c4)n3)c(-c3ccccc3)c2)cc1 ; c1ccc(-c2nc(-c3cccc(-c4cccc(-n5c6ccccc6c6cc7c(cc65)sc5ccccc57)c4)c3)nc(-c3cccc4c3sc3ccccc34)n2)cc1]

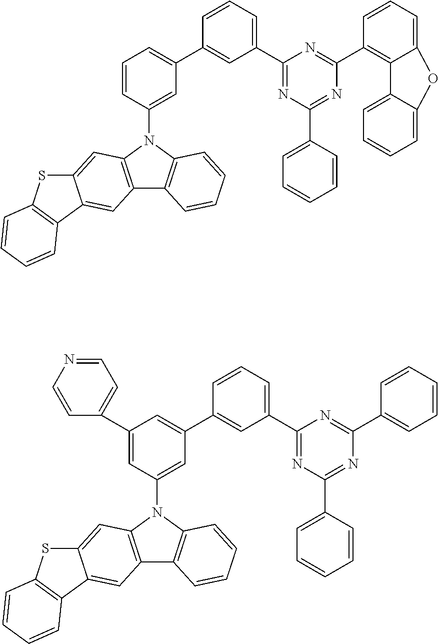 [SMILES: c1ccc(-c2nc(-c3cccc(-c4cccc(-n5c6ccccc6c6cc7c(cc65)sc5ccccc57)c4)c3)nc(-c3cccc4oc5ccccc5c34)n2)cc1 ; c1ccc(-c2nc(-c3ccccc3)nc(-c3cccc(-c4cc(-c5ccncc5)cc(-n5c6ccccc6c6cc7c(cc65)sc5ccccc57)c4)c3)n2)cc1]

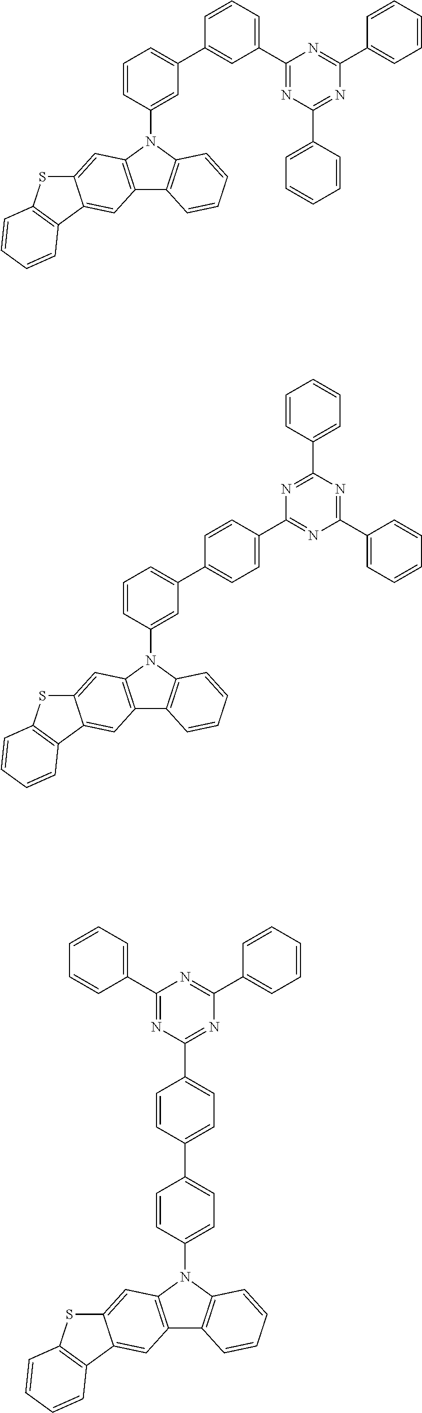 [SMILES: c1ccc(-c2nc(-c3ccccc3)nc(-c3cccc(-c4cccc(-n5c6ccccc6c6cc7c(cc65)sc5ccccc57)c4)c3)n2)cc1 ; c1ccc(-c2nc(-c3ccccc3)nc(-c3ccc(-c4cccc(-n5c6ccccc6c6cc7c(cc65)sc5ccccc57)c4)cc3)n2)cc1 ; c1ccc(-c2nc(-c3ccccc3)nc(-c3ccc(-c4ccc(-n5c6ccccc6c6cc7c(cc65)sc5ccccc57)cc4)cc3)n2)cc1]

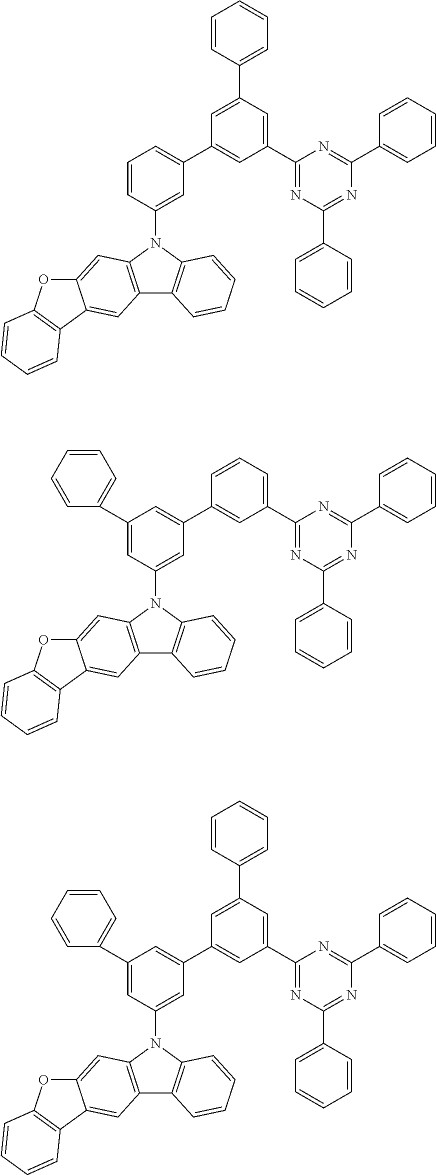 [SMILES: c1ccc(-c2cc(-c3cccc(-n4c5ccccc5c5cc6c(cc54)oc4ccccc46)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4cc5c(cc43)oc3ccccc35)c2)cc1 ; c1ccc(-c2cc(-c3cc(-c4ccccc4)cc(-n4c5ccccc5c5cc6c(cc54)oc4ccccc46)c3)cc(-c3nc(-c4ccccc4)nc(-c4ccccc4)n3)c2)cc1]

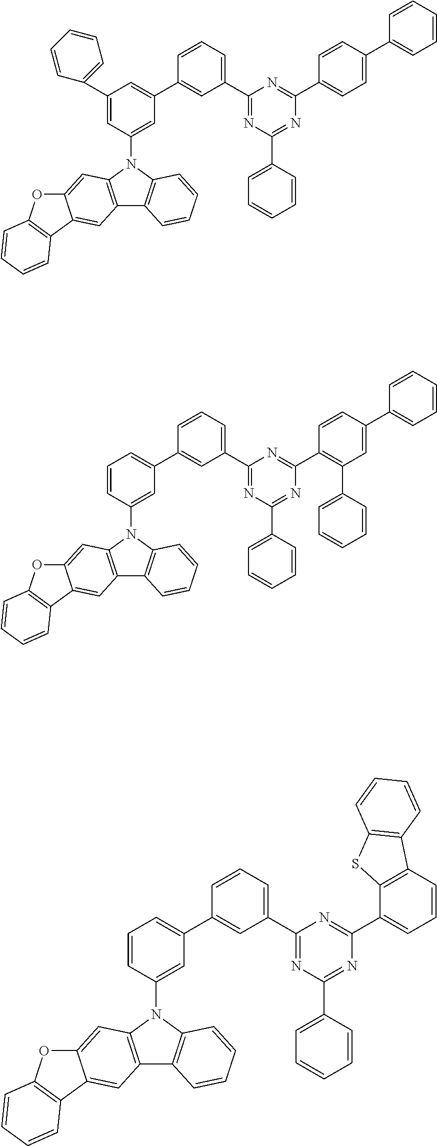 [SMILES: c1ccc(-c2ccc(-c3nc(-c4ccccc4)nc(-c4cccc(-c5cc(-c6ccccc6)cc(-n6c7ccccc7c7cc8c(cc76)oc6ccccc68)c5)c4)n3)cc2)cc1 ; c1ccc(-c2ccc(-c3nc(-c4ccccc4)nc(-c4cccc(-c5cccc(-n6c7ccccc7c7cc8c(cc76)oc6ccccc68)c5)c4)n3)c(-c3ccccc3)c2)cc1 ; c1ccc(-c2nc(-c3cccc(-c4cccc(-n5c6ccccc6c6cc7c(cc65)oc5ccccc57)c4)c3)nc(-c3cccc4c3sc3ccccc34)n2)cc1]

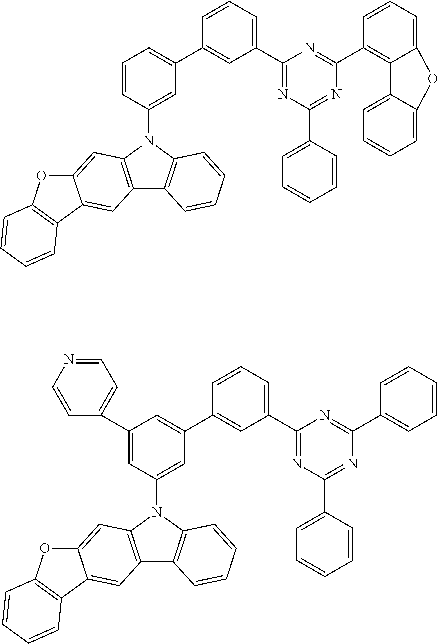 [SMILES: c1ccc(-c2nc(-c3cccc(-c4cccc(-n5c6ccccc6c6cc7c(cc65)oc5ccccc57)c4)c3)nc(-c3cccc4oc5ccccc5c34)n2)cc1 ; c1ccc(-c2nc(-c3ccccc3)nc(-c3cccc(-c4cc(-c5ccncc5)cc(-n5c6ccccc6c6cc7c(cc65)oc5ccccc57)c4)c3)n2)cc1]

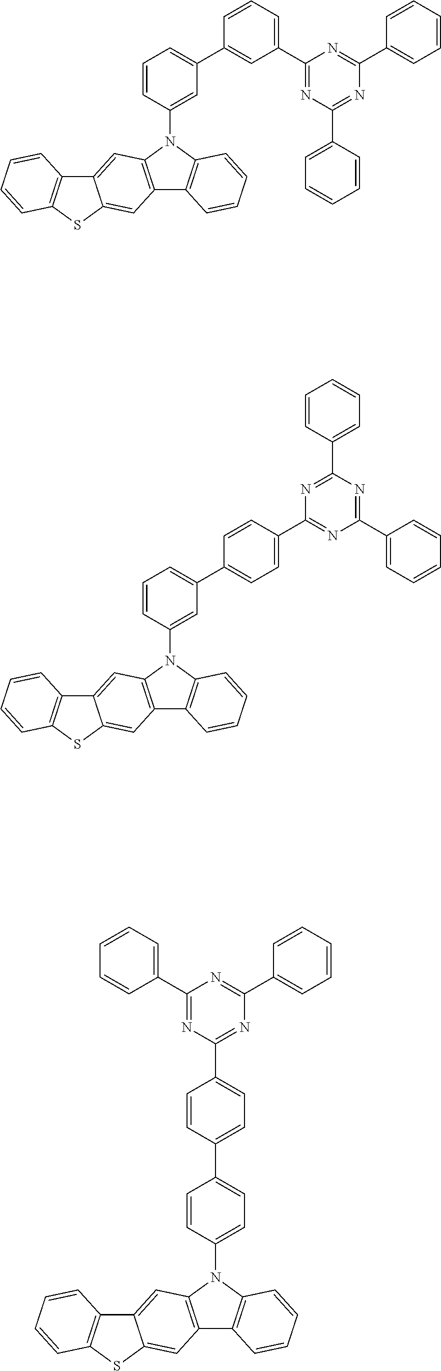 [SMILES: c1ccc(-c2nc(-c3ccccc3)nc(-c3cccc(-c4cccc(-n5c6ccccc6c6cc7sc8ccccc8c7cc65)c4)c3)n2)cc1 ; c1ccc(-c2nc(-c3ccccc3)nc(-c3ccc(-c4cccc(-n5c6ccccc6c6cc7sc8ccccc8c7cc65)c4)cc3)n2)cc1 ; c1ccc(-c2nc(-c3ccccc3)nc(-c3ccc(-c4ccc(-n5c6ccccc6c6cc7sc8ccccc8c7cc65)cc4)cc3)n2)cc1]

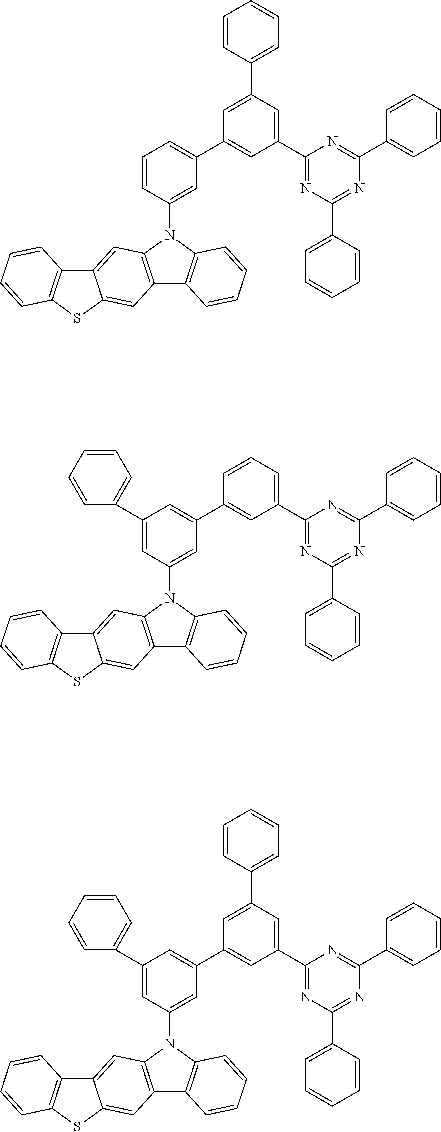 [SMILES: c1ccc(-c2cc(-c3cccc(-n4c5ccccc5c5cc6sc7ccccc7c6cc54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4cc5sc6ccccc6c5cc43)c2)cc1 ; c1ccc(-c2cc(-c3cc(-c4ccccc4)cc(-n4c5ccccc5c5cc6sc7ccccc7c6cc54)c3)cc(-c3nc(-c4ccccc4)nc(-c4ccccc4)n3)c2)cc1]

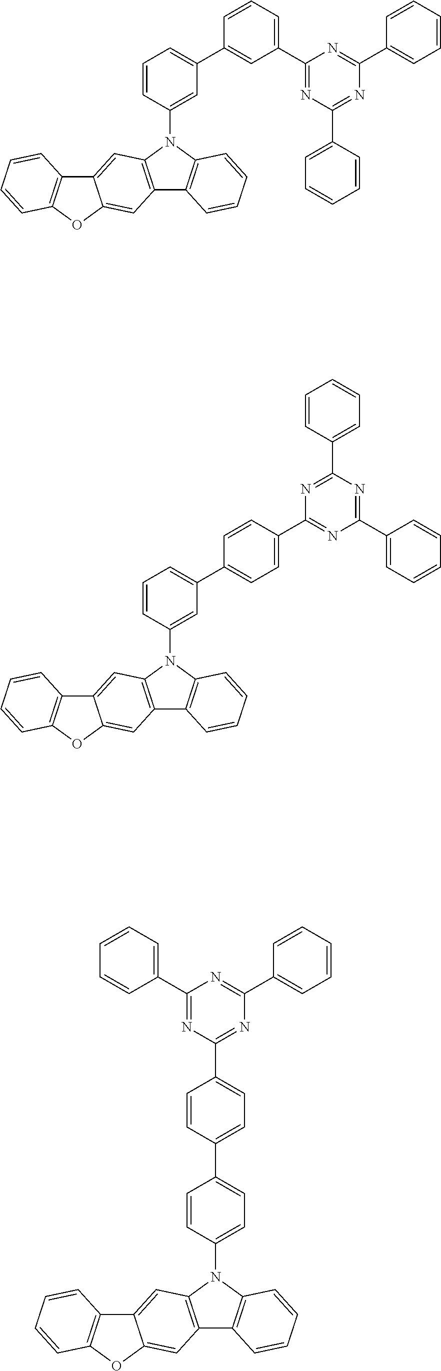 [SMILES: c1ccc(-c2nc(-c3ccccc3)nc(-c3cccc(-c4cccc(-n5c6ccccc6c6cc7oc8ccccc8c7cc65)c4)c3)n2)cc1 ; c1ccc(-c2nc(-c3ccccc3)nc(-c3ccc(-c4cccc(-n5c6ccccc6c6cc7oc8ccccc8c7cc65)c4)cc3)n2)cc1 ; c1ccc(-c2nc(-c3ccccc3)nc(-c3ccc(-c4ccc(-n5c6ccccc6c6cc7oc8ccccc8c7cc65)cc4)cc3)n2)cc1]

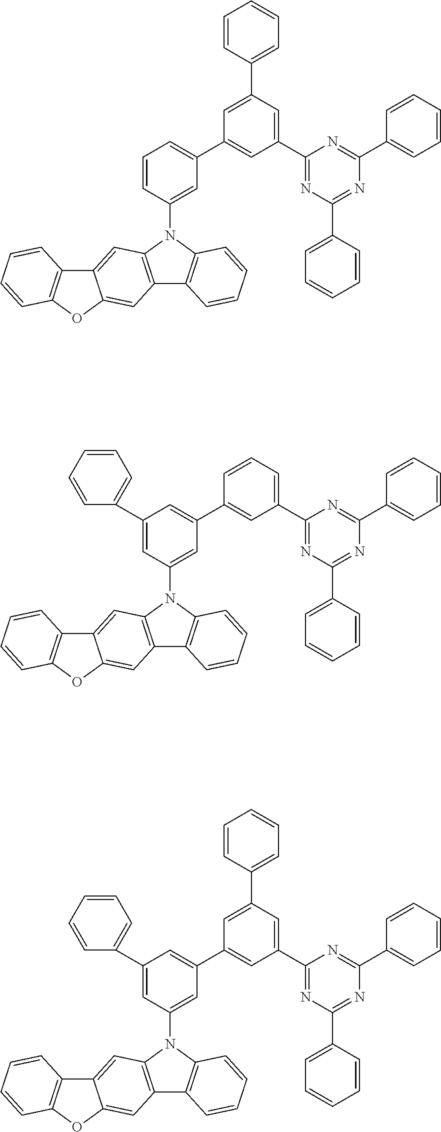 [SMILES: c1ccc(-c2cc(-c3cccc(-n4c5ccccc5c5cc6oc7ccccc7c6cc54)c3)cc(-c3nc(-c4ccccc4)nc(-c4ccccc4)n3)c2)cc1 ; c1ccc(-c2cc(-c3cccc(-c4nc(-c5ccccc5)nc(-c5ccccc5)n4)c3)cc(-n3c4ccccc4c4cc5oc6ccccc6c5cc43)c2)cc1 ; c1ccc(-c2cc(-c3cc(-c4ccccc4)cc(-n4c5ccccc5c5cc6oc7ccccc7c6cc54)c3)cc(-c3nc(-c4ccccc4)nc(-c4ccccc4)n3)c2)cc1]

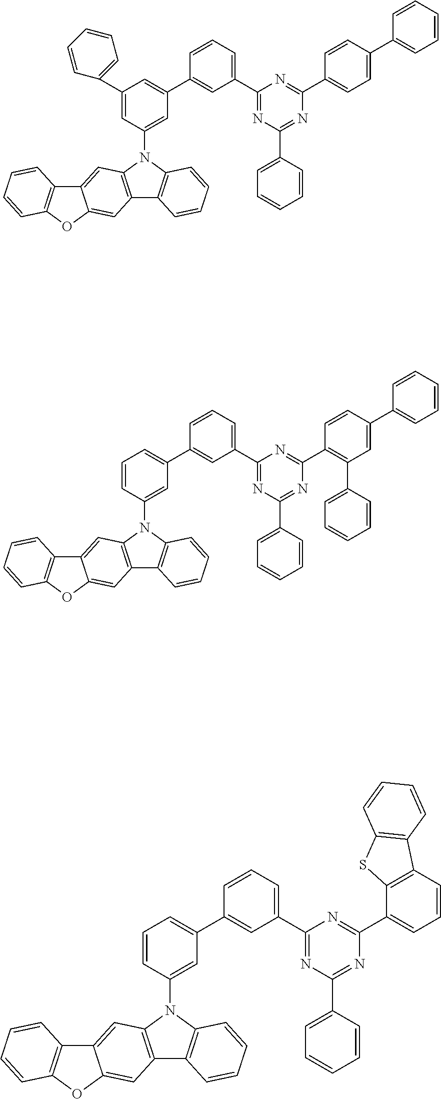 [SMILES: c1ccc(-c2ccc(-c3nc(-c4ccccc4)nc(-c4cccc(-c5cc(-c6ccccc6)cc(-n6c7ccccc7c7cc8oc9ccccc9c8cc76)c5)c4)n3)cc2)cc1 ; c1ccc(-c2ccc(-c3nc(-c4ccccc4)nc(-c4cccc(-c5cccc(-n6c7ccccc7c7cc8oc9ccccc9c8cc76)c5)c4)n3)c(-c3ccccc3)c2)cc1 ; c1ccc(-c2nc(-c3cccc(-c4cccc(-n5c6ccccc6c6cc7oc8ccccc8c7cc65)c4)c3)nc(-c3cccc4c3sc3ccccc34)n2)cc1]

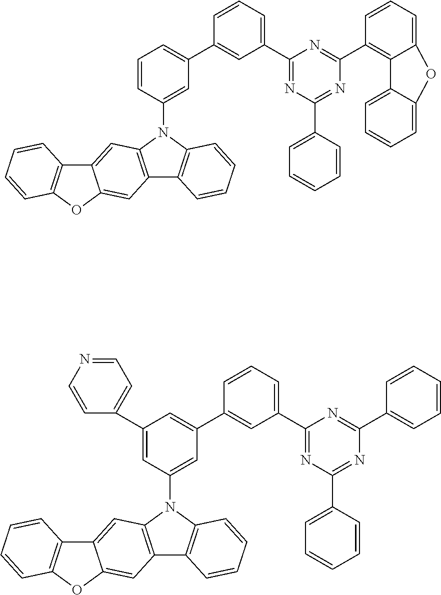 [SMILES: c1ccc(-c2nc(-c3cccc(-c4cccc(-n5c6ccccc6c6cc7oc8ccccc8c7cc65)c4)c3)nc(-c3cccc4oc5ccccc5c34)n2)cc1 ; c1ccc(-c2nc(-c3ccccc3)nc(-c3cccc(-c4cc(-c5ccncc5)cc(-n5c6ccccc6c6cc7oc8ccccc8c7cc65)c4)c3)n2)cc1]

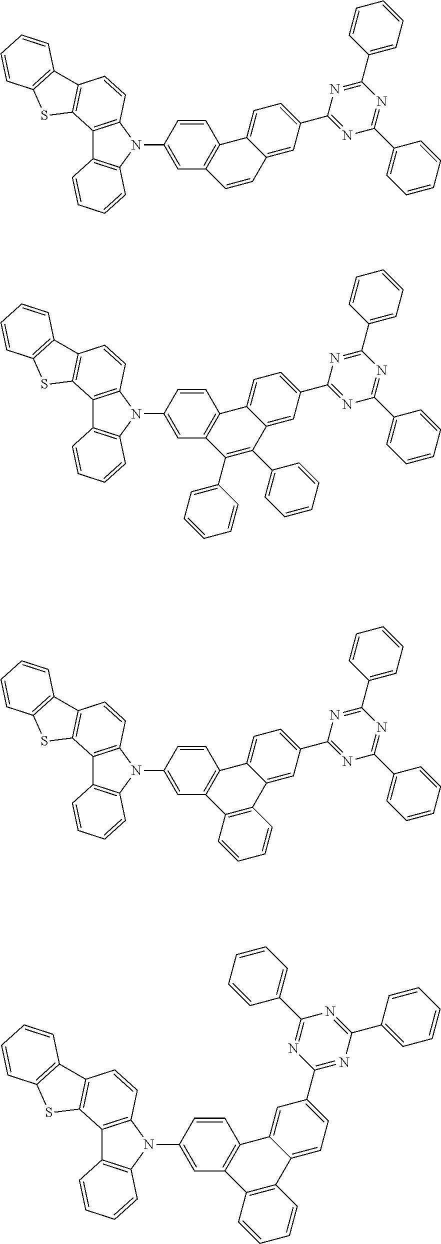 [SMILES: c1ccc(-c2nc(-c3ccccc3)nc(-c3ccc4c(ccc5cc(-n6c7ccccc7c7c8sc9ccccc9c8ccc76)ccc54)c3)n2)cc1 ; c1ccc(-c2nc(-c3ccccc3)nc(-c3ccc4c(c3)c(-c3ccccc3)c(-c3ccccc3)c3cc(-n5c6ccccc6c6c7sc8ccccc8c7ccc65)ccc34)n2)cc1 ; c1ccc(-c2nc(-c3ccccc3)nc(-c3ccc4c5ccc(-n6c7ccccc7c7c8sc9ccccc9c8ccc76)cc5c5ccccc5c4c3)n2)cc1 ; c1ccc(-c2nc(-c3ccccc3)nc(-c3ccc4c5ccccc5c5cc(-n6c7ccccc7c7c8sc9ccccc9c8ccc76)ccc5c4c3)n2)cc1]

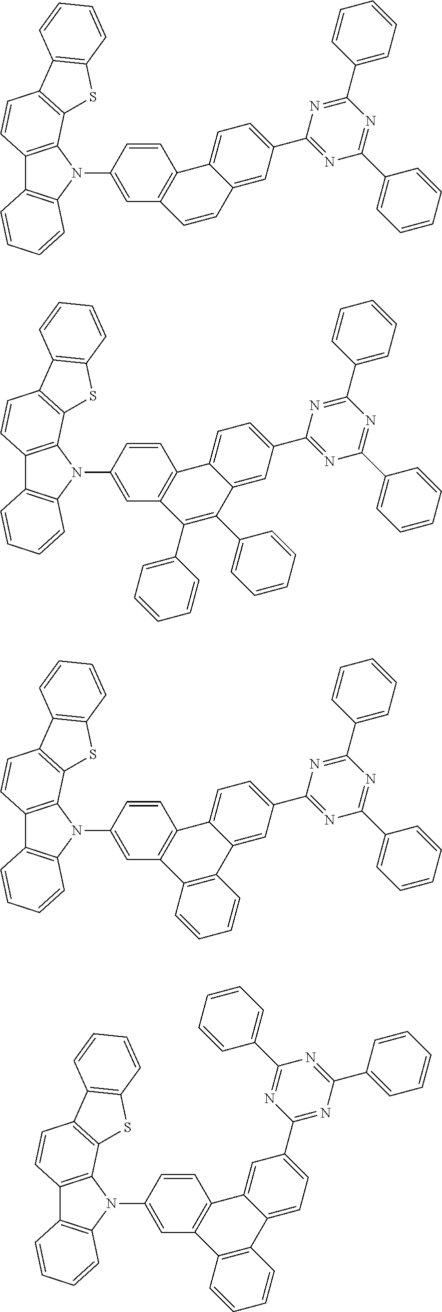 [SMILES: c1ccc(-c2nc(-c3ccccc3)nc(-c3ccc4c(ccc5cc(-n6c7ccccc7c7ccc8c9ccccc9sc8c76)ccc54)c3)n2)cc1 ; c1ccc(-c2nc(-c3ccccc3)nc(-c3ccc4c(c3)c(-c3ccccc3)c(-c3ccccc3)c3cc(-n5c6ccccc6c6ccc7c8ccccc8sc7c65)ccc34)n2)cc1 ; c1ccc(-c2nc(-c3ccccc3)nc(-c3ccc4c5ccc(-n6c7ccccc7c7ccc8c9ccccc9sc8c76)cc5c5ccccc5c4c3)n2)cc1 ; c1ccc(-c2nc(-c3ccccc3)nc(-c3ccc4c5ccccc5c5cc(-n6c7ccccc7c7ccc8c9ccccc9sc8c76)ccc5c4c3)n2)cc1]

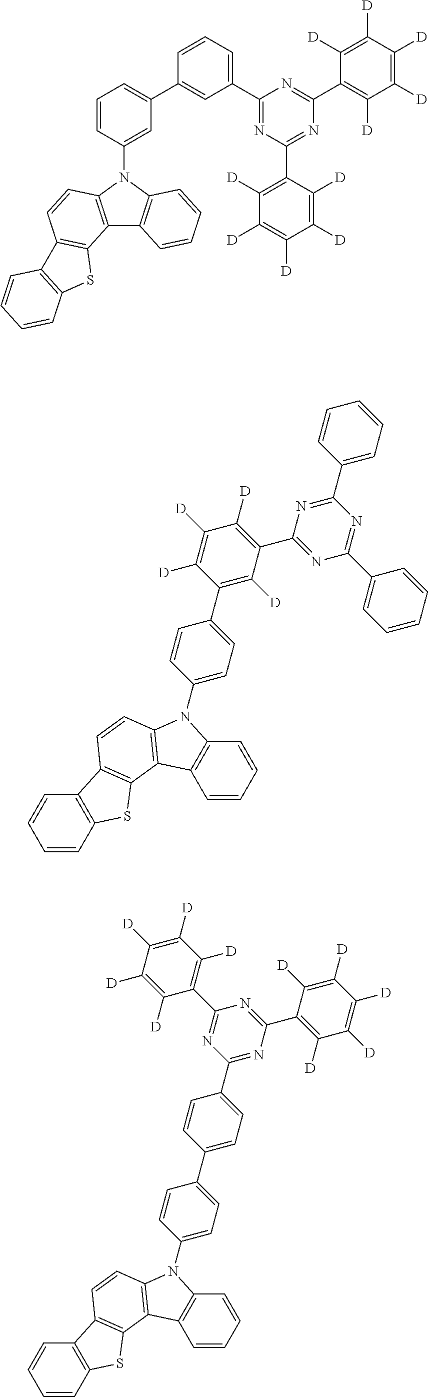 [SMILES: [2H]c1c([2H])c([2H])c(-c2nc(-c3cccc(-c4cccc(-n5c6ccccc6c6c7sc8ccccc8c7ccc65)c4)c3)nc(-c3c([2H])c([2H])c([2H])c([2H])c3[2H])n2)c([2H])c1[2H] ; [2H]c1c([2H])c(-c2ccc(-n3c4ccccc4c4c5sc6ccccc6c5ccc43)cc2)c([2H])c(-c2nc(-c3ccccc3)nc(-c3ccccc3)n2)c1[2H] ; [2H]c1c([2H])c([2H])c(-c2nc(-c3ccc(-c4ccc(-n5c6ccccc6c6c7sc8ccccc8c7ccc65)cc4)cc3)nc(-c3c([2H])c([2H])c([2H])c([2H])c3[2H])n2)c([2H])c1[2H]]

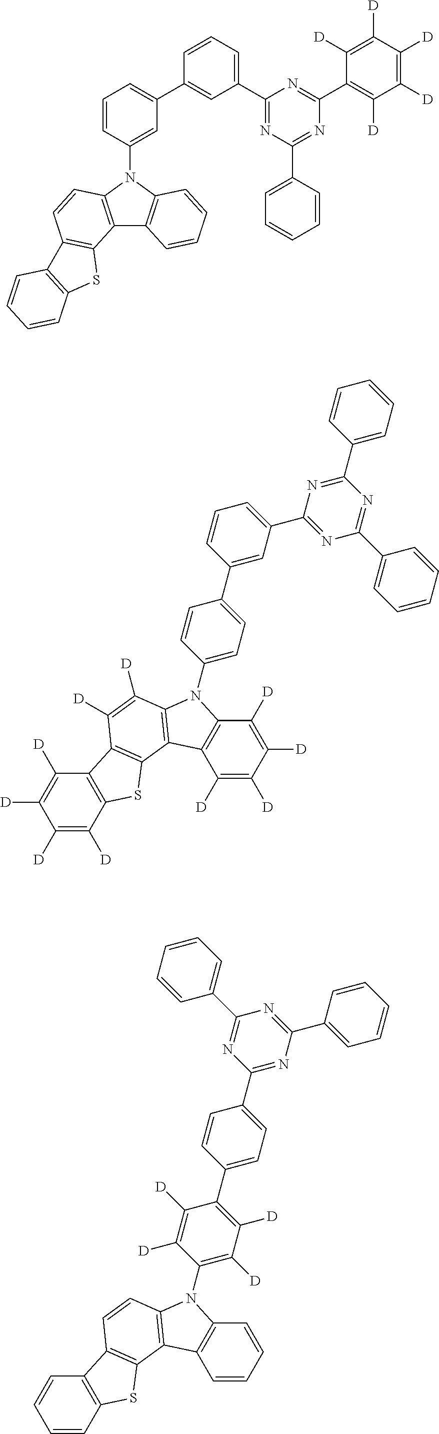 [SMILES: [2H]c1c([2H])c([2H])c(-c2nc(-c3ccccc3)nc(-c3cccc(-c4cccc(-n5c6ccccc6c6c7sc8ccccc8c7ccc65)c4)c3)n2)c([2H])c1[2H] ; [2H]c1c([2H])c([2H])c2c(sc3c2c([2H])c([2H])c2c3c3c([2H])c([2H])c([2H])c([2H])c3n2-c2ccc(-c3cccc(-c4nc(-c5ccccc5)nc(-c5ccccc5)n4)c3)cc2)c1[2H] ; [2H]c1c([2H])c(-n2c3ccccc3c3c4sc5ccccc5c4ccc32)c([2H])c([2H])c1-c1ccc(-c2nc(-c3ccccc3)nc(-c3ccccc3)n2)cc1]

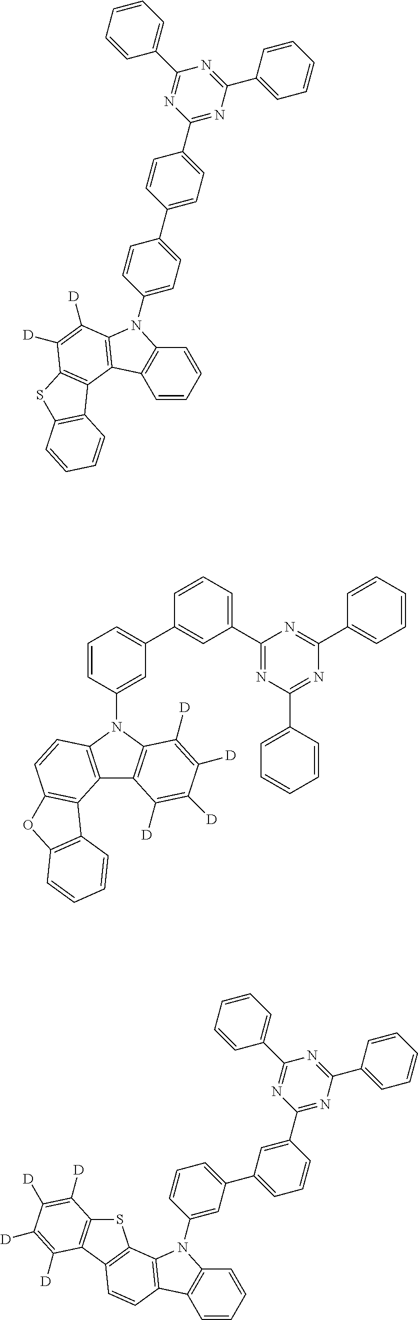 [SMILES: [2H]c1c([2H])c2c(c3ccccc3n2-c2ccc(-c3ccc(-c4nc(-c5ccccc5)nc(-c5ccccc5)n4)cc3)cc2)c2c1sc1ccccc12 ; [2H]c1c([2H])c([2H])c2c(c1[2H])c1c3c(ccc1n2-c1cccc(-c2cccc(-c4nc(-c5ccccc5)nc(-c5ccccc5)n4)c2)c1)oc1ccccc13 ; [2H]c1c([2H])c([2H])c2c(sc3c2ccc2c4ccccc4n(-c4cccc(-c5cccc(-c6nc(-c7ccccc7)nc(-c7ccccc7)n6)c5)c4)c23)c1[2H]]

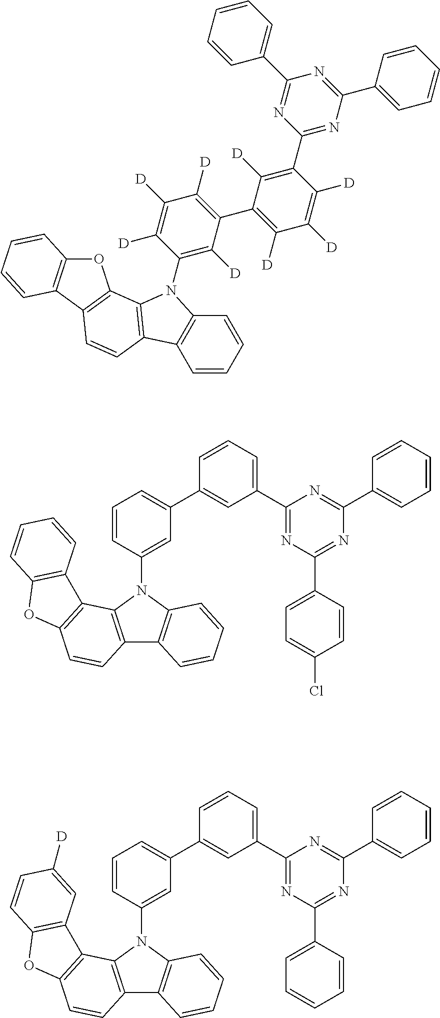 [SMILES: [2H]c1c([2H])c(-c2nc(-c3ccccc3)nc(-c3ccccc3)n2)c([2H])c(-c2c([2H])c([2H])c([2H])c(-n3c4ccccc4c4ccc5c6ccccc6oc5c43)c2[2H])c1[2H] ; Clc1ccc(-c2nc(-c3ccccc3)nc(-c3cccc(-c4cccc(-n5c6ccccc6c6ccc7oc8ccccc8c7c65)c4)c3)n2)cc1 ; [2H]c1ccc2oc3ccc4c5ccccc5n(-c5cccc(-c6cccc(-c7nc(-c8ccccc8)nc(-c8ccccc8)n7)c6)c5)c4c3c2c1]

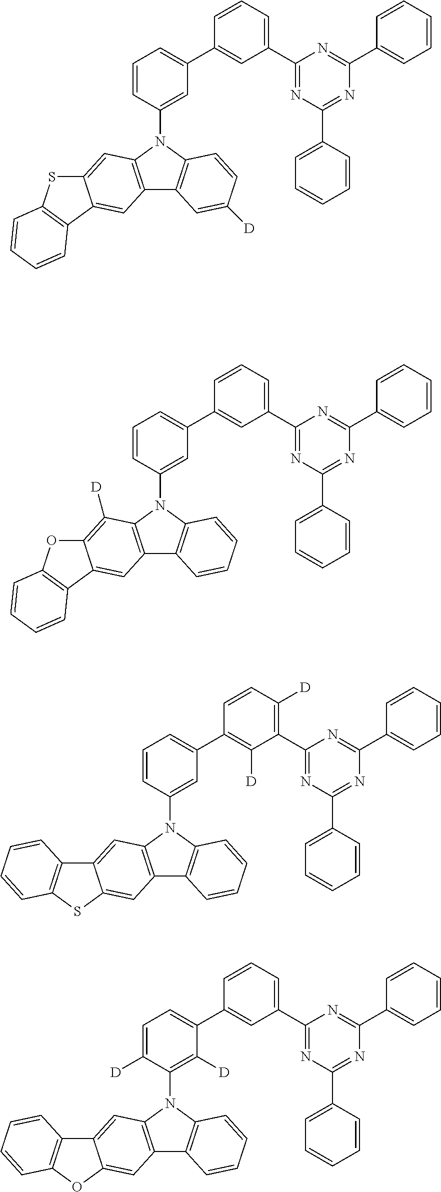 [SMILES: [2H]c1ccc2c(c1)c1cc3c(cc1n2-c1cccc(-c2cccc(-c4nc(-c5ccccc5)nc(-c5ccccc5)n4)c2)c1)sc1ccccc13 ; [2H]c1c2oc3ccccc3c2cc2c3ccccc3n(-c3cccc(-c4cccc(-c5nc(-c6ccccc6)nc(-c6ccccc6)n5)c4)c3)c12 ; [2H]c1ccc(-c2cccc(-n3c4ccccc4c4cc5sc6ccccc6c5cc43)c2)c([2H])c1-c1nc(-c2ccccc2)nc(-c2ccccc2)n1 ; [2H]c1ccc(-c2cccc(-c3nc(-c4ccccc4)nc(-c4ccccc4)n3)c2)c([2H])c1-n1c2ccccc2c2cc3oc4ccccc4c3cc21]

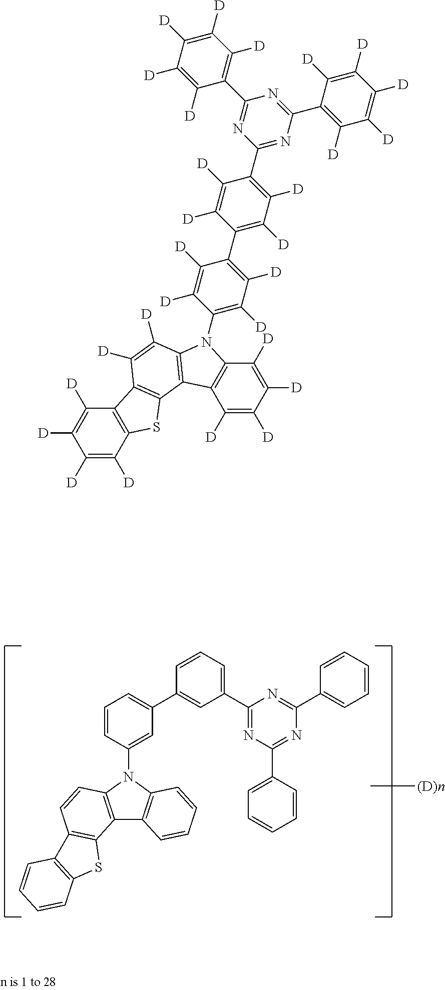

wherein (D), means that n of the hydrogen atoms in the compound is (are) a heavy hydrogen atom or heavy hydrogen atoms.

### Synthesis of the Compounds of Formulae (Ia) and (Ib)

The compounds of formulae (Ia) and (Ib) are for example prepared by one of the following processes:

### Process A:

A process for preparing a compound of formula (Ia) or (Ib), comprising the steps:

### (a) Coupling a Compound of Formula (Ia1) or (Ib1)

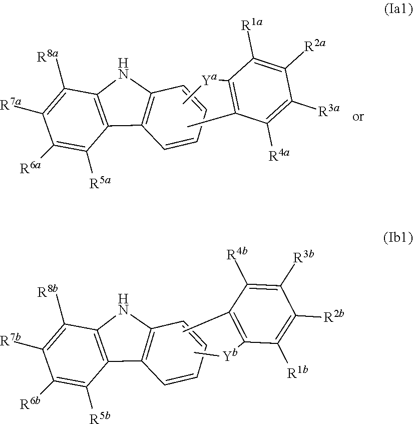

with a compound of formula (IIIa) or (IIIb)

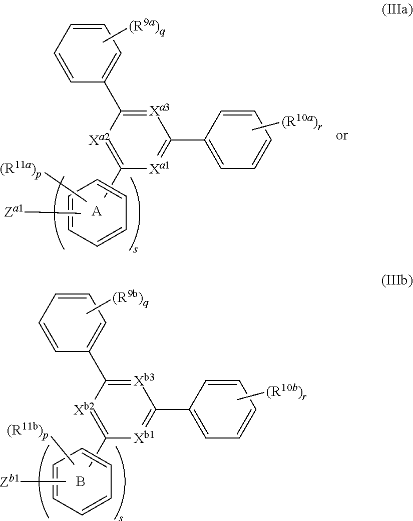

wherein

Z^{a1} and Z^{b1} each independently represents a halide, preferably selected from the group consisting of I, F, Cl and Br; a pseudohalide, preferably selected from the group consisting of mesylate, triflate, tosylate and nonaflate; —BQ₂, wherein Q is an unsubstituted alkyl group having 1 to 8 carbon atoms, an unsubstituted cycloalkyl group having 3 to 6 ring carbon atoms; a cycloalkyl group having 3 to 6 ring carbon atoms, substituted by one or two unsubstituted alkyl groups having 1 to 8 carbon atoms, a unsubstituted alkoxy group having 1 to 8 carbon atoms, a hydroxyl group, wherein two alkyl groups Q or two alkoxy groups Q together may form a five or six membered ring; or —MgX, wherein X is halide, whereby a compound of formula (Ia) or (Ib) is obtained

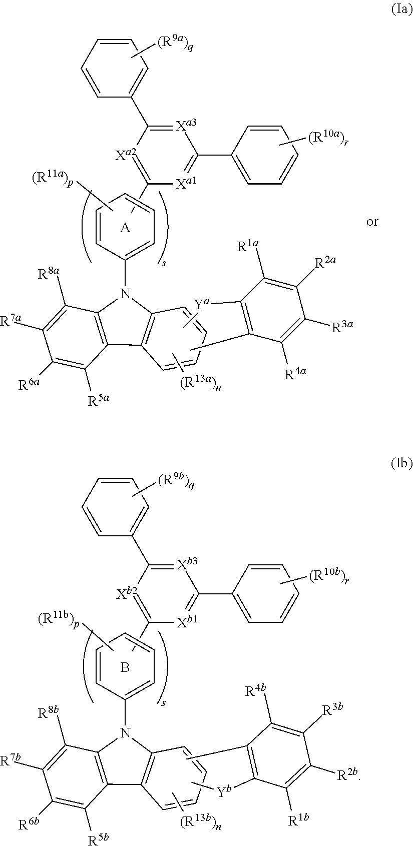

### Process B:

A process for preparing a compound of formula (Ia) or (Ib) comprising the steps:

(a) Coupling a compound of formula (IVa) or (IVb)

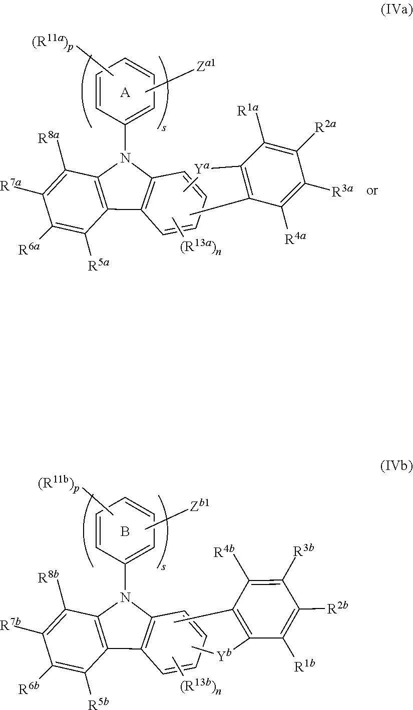

wherein

Z^{a1} and Z^{b1} each independently represents a halide, preferably selected from the group consisting of I, F, Cl and Br; a pseudohalide, preferably selected from the group consisting of mesylate, triflate, tosylate and nonaflate; —BQ₂, wherein Q is an unsubstituted alkyl group having 1 to 8 carbon atoms, an unsubstituted cycloalkyl group having 3 to 6 ring carbon atoms, a cycloalkyl group having 3 to 6 ring carbon atoms, substituted by one or two unsubstituted alkyl groups having 1 to 8 carbon atoms, a unsubstituted alkoxy group having 1 to 8 carbon atoms, a hydroxyl group, wherein two alkyl groups Q or two alkoxy groups Q together may form a five or six membered ring; or —MgX, wherein X is halide, with a compound of formula (Va) or (Vb)

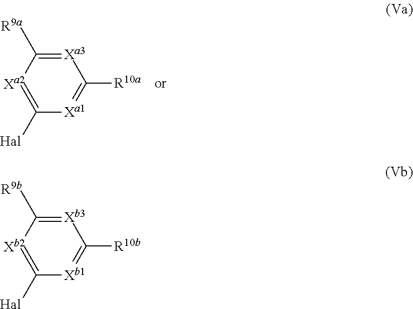

wherein

Hal is a halide, preferably selected from the group consisting of I, F, Cl and Br, or a pseudohalide, preferably selected from the group consisting of mesylate, triflate, tosylate and nonaflate,

whereby a compound of formula (Ia) or (Ib) is obtained

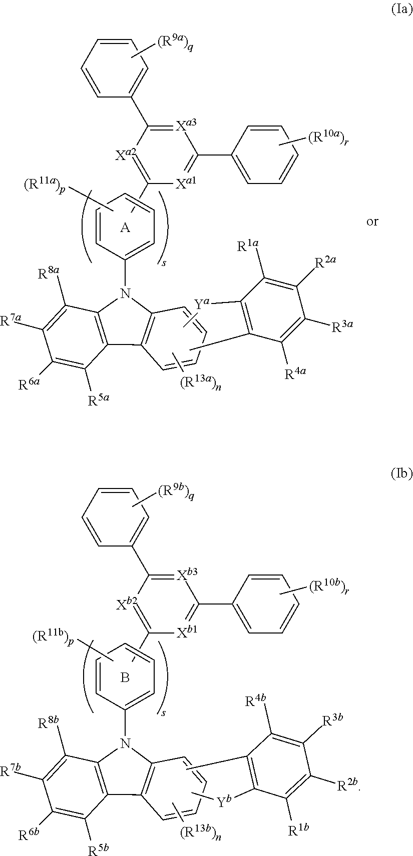

The groups, residues and indices R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{9a}, R^{10a}, R^{11a}, R^{13a}, X^{a1}, X^{a2}, X^{a3}, bra R^{9b}, R^{10b}, R^{11b}, R^{13b}, X^{b1}, X^{b2}, X^{b3}, Y^{b}, n, q, r, s and p are defined above.

Details of the reaction steps and process conditions are mentioned in the examples of the present application. The production method of the compounds of formula (Ia) and (Ib) according to the present invention is not particularly limited and it is produced according to known methods, for example, by a Suzuki coupling as described in Journal of American Chemistry Society, 1999, 121, 9550 to 9561 or Chemical Reviews, 1995, 95, 2457 to 2483 or Kumada coupling described in Org. Lett., 2010, 12, 2298-2301 or Angew. Chem., 2002, 114, 4218-4221. Coupling reactions involving a N atom and a C atom, so called amination reactions can be carried out in the presence of copper in an Ullmann type coupling such as described in Org. Lett., 2002, 4, 581-584 or Org. Lett., 2002, 4, 581-584 or a Chan Lam type coupling described in Org. Lett., 2003, 5, 4397-4400 or Org. Lett., 2013, 15, 1544-1547. Amination reactions can also be carried out in the presence of palladium in so-called Buchwald-Hartwig coupling such as described in Org. Lett., 2008, 10, 4109-4112, and Org. Lett., 2003, 5, 24b-2415. Amination reactions can also be carried out between amines and aryl fluorides be nucleophilic aromatic substitution such as described in Synthesis, 2016, 48(5), 737-750 or Angewandte Chemie, International Edition, 2012, 51(32), 8012-8016.

It has been found that the compounds of formulae (Ia) and (Ib) are particularly suitable for use in applications in which charge carrier conductivity is required, especially for use in organic electronics applications, for example selected from switching elements such as organic transistors, e.g. organic FETs and organic TFTs, organic solar cells and organic light-emitting diodes (OLEDs).

The term organic EL device (organic electroluminescence device) is used interchangeably with the term organic light-emitting diode (OLED) in the following; i.e. both terms have the same meaning in the sense of the present application.

The present invention further relates to a material for an organic EL device comprising at least one compound of formula (Ia) or (Ib).

The organic transistor generally includes a semiconductor layer formed from an organic layer with charge transport capacity; a gate electrode formed from a conductive layer; and an insulating layer introduced between the semiconductor layer and the conductive layer. A source electrode and a drain electrode are mounted on this arrangement in order thus to produce the transistor element. In addition, further layers known to those skilled in the art may be present in the organic transistor. The layers with charge transport capacity may comprise the compound of formula (Ia) or (Ib).

The organic solar cell (photoelectric conversion element) generally comprises an organic layer present between two plate-type electrodes arranged in parallel. The organic layer may be configured on a comb-type electrode. There is no particular restriction regarding the site of the organic layer and there is no particular restriction regarding the material of the electrodes. When, however, plate-type electrodes arranged in parallel are used, at least one electrode is preferably formed from a transparent electrode, for example an ITO electrode or a fluorine-doped tin oxide electrode. The organic layer is formed from two sublayers, i.e. a layer with p-type semiconductor properties or hole transport capacity, and a layer formed with n-type semiconductor properties or charge transport capacity. In addition, it is possible for further layers known to those skilled in the art to be present in the organic solar cell. The layers with charge transport capacity may comprise the compound of formula (Ia) or (Ib).

The compounds of formulae (Ia) and (Ib) are particularly suitable in OLEDs for use as charge and/or exciton-blocking material, i.e. as electron/exciton-blocking material or as hole/exciton-blocking material, and/or charge-transporting material, i.e. hole-transporting material or electron-transporting material, preferably as electron-transporting material and/or hole-blocking material.

In the case of use of the inventive compounds of formulae (Ia) and (Ib) in OLEDs, OLEDs having good overall properties, preferably a long lifetime, high external quantum efficiencies (EQE) and/or a low driving voltage are obtained.

### Organic Electroluminescence Device

According to one aspect of the present invention, a material for an organic electroluminescence device, comprising at least one compound of formula (Ia) or (Ib) is provided.

According to another aspect of the invention, the following organic electroluminescence device is provided, comprising at least one compound of formula (Ia) or (Ib). The organic electroluminescence device generally comprises: a cathode, an anode, and one or more organic thin film layers comprising an emitting layer (also referred to as “light emitting layer”) disposed between the cathode and the anode, wherein at least one layer of the organic thin film layers comprises at least one compound of formula (Ia) or (Ib).

In the present specification, regarding “one or more organic thin film layers disposed between the cathode and the anode”, if only one organic layer is present between the cathode and the anode, it means the layer, and if plural organic layers are present between the cathode and the anode, it means at least one layer thereof.

According to another aspect of the invention, the use of a compound of formula (Ia) or (Ib) according to the present invention in an organic electroluminescence device is provided.

In one embodiment, the organic EL device has a hole-transporting layer between the anode and the emitting layer.

In one embodiment, the organic EL device has an electron-transporting layer between the cathode and the emitting layer.

In one embodiment, the organic EL device has a hole-blocking layer between the electron-transporting layer and the emitting layer.

### Layer(s) Between the Emitting Layer and the Anode:

In the organic EL device according to the present invention, one or more organic thin film layers may be present between the emitting layer and the anode. If only one organic layer is present between the emitting layer and the anode, it means that layer, and if plural organic layers are present, it means at least one layer thereof. For example, if two or more organic layers are present between the emitting layer and the anode, an organic layer nearer to the emitting layer is called the “hole-transporting layer”, and an organic layer nearer to the anode is called the “hole-injecting layer”. Each of the “hole-transporting layer” and the “hole injecting layer” may be a single layer or may be formed of two or more layers. One of these layers may be a single layer and the other may be formed of two or more layers.

### Layer(s) Between the Emitting Layer and the Cathode:

Similarly, one or more organic thin film layers may be present between the emitting layer and the cathode, in the organic EL device according to the present invention (electron-transporting zone, at least including an electron-transporting layer and preferably also an electron-injecting layer and/or a hole-blocking layer). If only one organic layer is present between the emitting layer and the cathode it means that layer, and if plural organic layers are present, it means at least one layer thereof. For example, if two or more organic layers are present between the emitting layer and the cathode, an organic layer nearest to the emitting layer is called the “hole-blocking layer”, one organic layer nearest to the “hole-blocking layer” is called the “electron-transporting layer”, and an organic layer nearer to the cathode is called the “electron-injecting layer”. Each of the “hole-blocking layer”, “electron-transporting layer” and the “electron-injecting layer” may be a single layer or may be formed of two or more layers. One of these layers may be a single layer and the other may be formed of two or more layers.

The one or more organic thin film layers between the emitting layer and the cathode, preferably the “electron-transporting zone”, preferably comprises a compound represented by formula (Ia) or (Ib).

Therefore, in a preferred embodiment, the organic thin film layers of the organic electroluminescence device comprise an electron-transporting zone provided between the emitting layer and the cathode, wherein the electron-transporting zone comprises at least one compound represented by formula (Ia) or (Ib). The compound represented by formula (Ia) or (Ib) preferably functions as “hole-blocking” material in the hole-blocking layer and/or “electron-transporting” material in the electron-transporting layer.

In an exemplary embodiment, the one or more organic thin film layers of the organic EL device of the present invention at least include the emitting layer and an electron-transporting zone. The electron-transporting zone is provided between the emitting layer and the cathode and at least includes an electron-transporting layer and preferably also an electron injecting layer and/or a hole-blocking layer. The electron-transporting zone may include the electron-injecting layer and an electron-transporting layer and may further include a hole-blocking layer and optionally a space layer. In addition to the above layers, the one or more organic thin film layers may be provided by layers applied in a known organic EL device such as a hole-injecting layer, a hole transporting layer and an electron-blocking layer. The one or more organic thin film layers may include an inorganic compound.

An explanation will be made on the layer configuration of the organic EL device according to one aspect of the invention.

An organic EL device according to one aspect of the invention comprises a cathode, an anode, and one or more organic thin film layers comprising an emitting layer disposed between the cathode and the anode. The organic layer comprises at least one layer composed of an organic compound. Alternatively, the organic layer is formed by laminating a plurality of layers composed of an organic compound. The organic layer may further comprise an inorganic compound in addition to the organic compound.

At least one of the organic layers is an emitting layer. The organic layer may be constituted, for example, as a single emitting layer, or may comprise other layers which can be adopted in the layer structure of the organic EL device. The layer that can be adopted in the layer structure of the organic EL device is not particularly limited, but examples thereof include a hole-transporting zone (a hole-transporting layer, a hole-injecting layer, an electron-blocking layer, an exciton-blocking layer, etc.), an emitting layer, a spacing layer, and an electron-transporting zone (electron-transporting layer, electron-injecting layer, hole-blocking layer, etc.) provided between the cathode and the emitting layer.

The organic EL device according to one aspect of the invention may be, for example, a fluorescent or phosphorescent monochromatic light emitting device or a fluorescent/phosphorescent hybrid white light emitting device.

Further, it may be a simple type device having a single emitting unit or a tandem type device having a plurality of emitting units.

The “emitting unit” in the specification is the smallest unit that comprises organic layers, in which at least one of the organic layers is an emitting layer and light is emitted by recombination of injected holes and electrons.

In addition, the emitting layer described in the present specification is an organic layer having an emitting function. The emitting layer is, for example, a phosphorescent emitting layer, a fluorescent emitting layer or the like, and may be a single layer or a stack of a plurality of layers.

The “emitting unit” may be a stacked type unit having a plurality of phosphorescent emitting layers or fluorescent emitting layers. In this case, for example, a spacing layer for preventing excitons generated in the phosphorescent emitting layer from diffusing into the fluorescent emitting layer may be provided between the respective light-emitting layers.

As the simple type organic EL device, a device configuration such as anode/emitting unit/cathode can be given.

Examples for representative layer structures of the emitting unit are shown below. The layers in parentheses are provided arbitrarily:

(a) (Hole-injecting layer/) Hole-transporting layer/Fluorescent emitting layer (/Electron-transporting layer/Electron-injecting layer)
(b) (Hole-injecting layer/) Hole-transporting layer/Phosphorescent emitting layer (/Electron-transporting layer/Electron-injecting layer)
(c) (Hole-injecting layer/) Hole-transporting layer/First fluorescent emitting layer/Second fluorescent emitting layer (/Electron-transporting layer/Electron-injecting layer)
(d) (Hole-injecting layer/) Hole-transporting layer/First phosphorescent layer/Second phosphorescent layer (/Electron-transporting layer/Electron-injecting layer)
(e) (Hole-injecting layer/) Hole-transporting layer/Phosphorescent emitting layer/Spacing layer/Fluorescent emitting layer (/Electron-transporting layer/Electron-injecting layer)
(f) (Hole-injecting layer/) Hole-transporting layer/First phosphorescent emitting layer/Second phosphorescent emitting layer/Spacing layer/Fluorescent emitting layer (/Electron-transporting layer/Electron-injecting layer)
(g) (Hole-injecting layer/) Hole-transporting layer/First phosphorescent layer/Spacing layer/Second phosphorescent emitting layer/Spacing layer/Fluorescent emitting layer (/Electron-transporting layer/Electron-injecting layer)
(h) (Hole-injecting layer/) Hole-transporting layer/Phosphorescent emitting layer/Spacing layer/First fluorescent emitting layer/Second fluorescent emitting layer (/Electron-transporting Layer/Electron-injecting Layer)
(i) (Hole-injecting layer/) Hole-transporting layer/Electron-blocking layer/Fluorescent emitting layer (/Electron-transporting layer/Electron-injecting layer)
(j) (Hole-injecting layer/) Hole-transporting layer/Electron-blocking layer/Phosphorescent emitting layer (/Electron-transporting layer/Electron-injecting layer)
(k) (Hole-injecting layer/) Hole-transporting layer/Exciton-blocking layer/Fluorescent emitting layer (/Electron-transporting layer/Electron-injecting layer)
(l) (Hole-injecting layer/) Hole-transporting layer/Exciton-blocking layer/Phosphorescent emitting layer (/Electron-transporting layer/Electron-injecting layer)
(m) (Hole-injecting layer/) First hole-transporting Layer/Second hole-transporting Layer/Fluorescent emitting layer (/Electron-transporting layer/electron-injecting Layer)
(n) (Hole-injecting layer/) First hole-transporting layer/Second hole-transporting layer/Fluorescent emitting layer (/First electron-transporting layer/Second electron-transporting layer/Electron-injection layer)
(o) (Hole-injecting layer/) First hole-transporting layer/Second hole-transporting layer/Phosphorescent emitting layer (/Electron-transporting layer/Electron-injecting Layer)
(p) (Hole-injecting layer/) First hole-transporting layer/Second hole-transporting layer/Phosphorescent emitting layer (/First electron-transporting Layer/Second electron-transporting layer/Electron-injecting layer)
(q) (Hole-injecting layer/) Hole-transporting layer/Fluorescent emitting layer/Hole-blocking layer (/Electron-transporting layer/Electron-injecting layer)
(r) (Hole-injecting layer/) Hole-transporting layer/Phosphorescent emitting layer/Hole-blocking layer (/Electron-transport layer/Electron-injecting layer)
(s) (Hole-injecting layer/) Hole-transporting layer/Fluorescent emitting layer/Exciton-blocking layer (/Electron-transporting layer/Electron-injecting layer)
(t) (Hole-injecting layer/) Hole-transporting layer/Phosphorescent emitting layer/Exciton-blocking layer (/Electron-transporting layer/Electron-injecting layer)

The layer structure of the organic EL device according to one aspect of the invention is not limited to the examples mentioned above.

For example, when the organic EL device has a hole-injecting layer and a hole-transporting layer, it is preferred that a hole-injecting layer be provided between the hole-transporting layer and the anode. Further, when the organic EL device has an electron-injecting layer and an electron-transporting layer, it is preferred that an electron-injecting layer be provided between the electron-transporting layer and the cathode. Further, each of the hole-injecting layer, the hole-transporting layer, the electron-transporting layer and the electron-injecting layer may be formed of a single layer or be formed of a plurality of layers.

The plurality of phosphorescent emitting layers and/or fluorescent emitting layers may be emitting layers that emit mutually different colors. For example, the emitting unit (f) may include a hole-transporting layer/first phosphorescent layer (red light emission)/second phosphorescent emitting layer (green light emission)/spacing layer/fluorescent emitting layer (blue light emission)/electron-transporting layer.

An electron-blocking layer may be provided between each light emitting layer and the hole-transporting layer or the spacing layer. Further, a hole-blocking layer may be provided between each emitting layer and the electron-transporting layer. By providing the electron-blocking layer or the hole-blocking layer, it is possible to confine electrons or holes in the emitting layer, thereby to improve the recombination probability of carriers in the emitting layer, and to improve light emitting efficiency.

As a representative device configuration of a tandem type organic EL device, for example, a device configuration such as anode/first emitting unit/intermediate layer/second emitting unit/cathode can be given:

The first emitting unit and the second emitting unit are independently selected from the above-mentioned emitting units, for example.

The intermediate layer is also generally referred to as an intermediate electrode, an intermediate conductive layer, a charge generating layer, an electron withdrawing layer, a connecting layer, a connector layer, or an intermediate insulating layer. The intermediate layer is a layer that supplies electrons to the first emitting unit and holes to the second emitting unit, and can be formed from known materials.

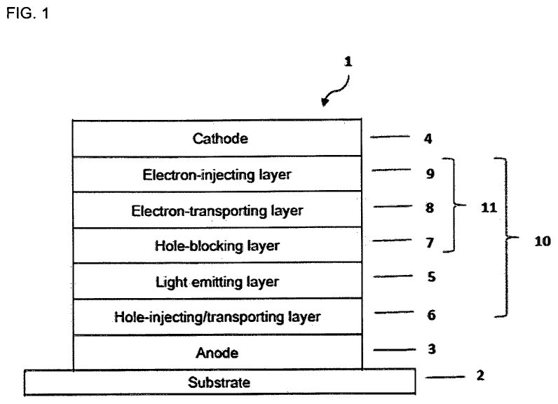

FIG. 1 shows a schematic configuration of one example of the organic EL device of the invention. The organic EL device 1 comprises a substrate 2, an anode 3, a cathode 4 and an emitting unit 10 provided between the anode 3 and the cathode 4. The emitting unit 10 comprises a light emitting layer 5 preferably comprising a host material and a dopant. A hole-injecting/transporting layer 6 or the like may be provided between the light emitting layer 5 and the anode 3 and an electron-injecting layer 9 and an electron-transporting layer 8 and/or a hole-blocking layer 7 or the like (electron-transporting zone 11) may be provided between the light emitting layer 5 and the cathode 4. An electron-blocking layer may be provided on the anode 3 side of the light emitting layer 5. Due to such configuration, electrons or holes can be confined in the light emitting layer 5, whereby possibility of generation of excitons in the light emitting layer 5 can be improved.

Hereinbelow, an explanation will be made on function, materials, etc. of each layer constituting the organic EL device described in the present specification.

### Substrate

The substrate is used as a support of the organic EL device. The substrate preferably has a light transmittance of 50% or more in the visible light region with a wavelength of 400 to 700 nm, and a smooth substrate is preferable. Examples of the material of the substrate include soda-lime glass, aluminosilicate glass, quartz glass, plastic and the like. As a substrate, a flexible substrate can be used. The flexible substrate means a substrate that can be bent (flexible), and examples thereof include a plastic substrate and the like. Specific examples of the material for forming the plastic substrate include polycarbonate, polyallylate, polyether sulfone, polypropylene, polyester, polyvinyl fluoride, polyvinyl chloride, polyimide, polyethylene naphthalate and the like. Also, an inorganic vapor deposited film can be used.

### Anode

As the anode, for example, it is preferable to use a metal, an alloy, a conductive compound, a mixture thereof or the like and having a high work function (specifically, 4.0 eV or more). Specific examples of the material of the anode include indium oxide-tin oxide (ITO: Indium Tin Oxide), indium oxide-tin oxide containing silicon or silicon oxide, indium oxide-zinc oxide, indium oxide containing tungsten oxide or zinc oxide, graphene and the like. In addition, it is also possible to use gold, silver, platinum, nickel, tungsten, chromium, molybdenum, iron, cobalt, copper, palladium, titanium, and nitrides of these metals (e.g. titanium oxide).

The anode is normally formed by depositing these materials on the substrate by a sputtering method. For example, indium oxide-zinc oxide can be formed by a sputtering method by using a target in which 1 to 10 mass % zinc oxide is added relative to indium oxide. Further, indium oxide containing tungsten oxide or zinc oxide can be formed by a sputtering method by using a target in which 0.5 to 5 mass % of tungsten oxide or 0.1 to 1 mass % of zinc oxide is added relative to indium oxide.

As other methods for forming the anode, a vacuum deposition method, a coating method, an inkjet method, a spin coating method or the like can be given. When silver paste or the like is used, it is possible to use a coating method, an inkjet method or the like.

The hole-injecting layer formed in contact with the anode is formed by using a material that allows easy hole injection regardless of the work function of the anode. For this reason, in the anode, it is possible to use a common electrode material, e.g. a metal, an alloy, a conductive compound and a mixture thereof. Specifically, a material having a small work function such as alkaline metals such as lithium and cesium; alkaline earth metals such as calcium and strontium; alloys containing these metals (for example, magnesium-silver and aluminum-lithium); rare earth metals such as europium and ytterbium; and an alloy containing rare earth metals.

### Hole-Transporting Layer, Hole-Injecting Layer, Electron-Blocking Layer

The hole-transporting layer is an organic layer that is formed between the emitting layer and the anode, and has a function of transporting holes from the anode to the emitting layer. If the hole-transporting layer is composed of plural layers, an organic layer that is nearer to the anode may often be defined as the hole-injecting layer. The hole-injecting layer has a function of injecting holes efficiently to the organic layer unit from the anode. Said hole-injecting layer is generally used for stabilizing hole injection from anode to hole-transporting layer which is generally consist of organic materials. Organic material having good contact with anode or organic material with p-type doping is preferably used for the hole-injecting layer.

p-doping usually consists of one or more p-dopant materials and one or more matrix materials. Matrix materials preferably have shallower HOMO level and p-dopant preferably have deeper LUMO level to enhance the carrier density of the layer. Aryl or heteroaryl amine compounds are preferably used as the matrix materials. Specific examples for the matrix material are the same as that for hole-transporting layer which is explained at the later part. Specific examples for p-dopant are the below mentioned acceptor materials, preferably the quinone compounds with one or more electron withdrawing groups, such as F₄TCNQ, 1,2,3-tris[(cyano)(4-cyano-2,3,5,6-tetrafluorophenyl)methylene]cyclopropane.

Acceptor materials, or fused aromatic hydrocarbon materials or fused heterocycles which have high planarity are preferably used as p-dopant materials for the hole-injecting layer. Specific examples for acceptor materials are the quinone compounds with one or more electron withdrawing groups, such as F₄TCNQ(2,3,5,6-tetrafluoro-7,7,8,8-tetracyanoquinodimethane), and 1,2,3-tris[(cyano)(4-cyano-2,3,5,6-tetrafluorophenyl)methylene]cyclopropane; hexa-azatriphenylene compounds with one or more electron withdrawing groups, such as hexa-azatriphenylene-hexanitrile; aromatic hydrocarbon compounds with one or more electron withdrawing groups; and aryl boron compounds with one or more electron withdrawing groups.

The ratio of the p-type dopant is preferably less than 20% of molar ratio, more preferably less than 10%, such as 1%, 3%, or 5%, related to the matrix material.

The hole-transporting layer is generally used for injecting and transporting holes efficiently, and aromatic or heterocyclic amine compounds are preferably used.

Specific examples for compounds for the hole-transporting layer are represented by the general formula (H):

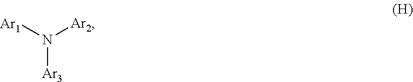

wherein

Ar₁ to Ar₃ each independently represents substituted or unsubstituted aryl group having 5 to 50 ring carbon atoms or substituted or unsubstituted heterocyclic group having 5 to 50 ring atoms, preferably phenyl group, biphenyl group, terphenyl group, naphthyl group, phenanthryl group, triphenylenyl group, fluorenyl group, spirobifluorenyl group, indenofluorenyl group, carbazolyl group, dibenzofuranyl group, dibenzothiophenyl group, carbazole substituted aryl group, dibenzofuran substituted aryl group or dibenzothiophene substituted aryl group; two or more substituents selected among Ar¹ to Ar³ may be bonded to each other to form a ring structure, such as a carbazole ring structure, or a acridan ring structure.

Preferably, at least one of Ar₁ to Ar₃ have additional one aryl or heterocyclic amine substituent, more preferably Ar₁ has an additional aryl amino substituent, at the case of that it is preferable that Ar₁ represents substituted or unsubstituted biphenylene group, substituted or unsubstituted fluorenylene group.

A second hole-transporting layer is preferably inserted between the first hole-transporting layer and the emitting layer to enhance device performance by blocking excess electrons or excitons.

Specific examples for second hole-transporting layer are the same as for the first hole-transporting layer. It is preferred that second hole-transporting layer has higher triplet energy to block triplet excitons, especially for phosphorescent green device, such as bicarbazole compounds, biphenylamine compounds, triphenylenyl amine compounds, fluorenyl amine compounds, carbazole substituted arylamine compounds, dibenzofuran substituted arylamine compounds, and dibenzothiophene substituted arylamine compounds.

This second hole-transporting layer also called electron-blocking layer provided adjacent to the emitting layer has a function of preventing leakage of electrons from the emitting layer to the hole-transporting layer.

### Emitting Layer

The emitting layer is a layer containing a substance having a high emitting property (emitter material or dopant material). As the dopant material, various materials can be used. For example, a fluorescent emitting compound (fluorescent dopant), a phosphorescent emitting compound (phosphorescent dopant) or the like can be used. A fluorescent emitting compound is a compound capable of emitting light from the singlet excited state, and an emitting layer containing a fluorescent emitting compound is called a fluorescent emitting layer. Further, a phosphorescent emitting compound is a compound capable of emitting light from the triplet excited state, and an emitting layer containing a phosphorescent emitting compound is called a phosphorescent emitting layer.

Preferably, the emitting layer in the organic EL device of the present application comprises a compound of formula (Ia) or (Ib) as a dopant material.

The emitting layer preferably comprises at least one dopant material and at least one host material that allows it to emit light efficiently. In some literatures, a dopant material is called a guest material, an emitter or an emitting material. In some literatures, a host material is called a matrix material.

A single emitting layer may comprise plural dopant materials and plural host materials. Further, plural emitting layers may be present.

In the present specification, a host material combined with the fluorescent dopant is referred to as a “fluorescent host” and a host material combined with the phosphorescent dopant is referred to as the “phosphorescent host”. Note that the fluorescent host and the phosphorescent host are not classified only by the molecular structure. The phosphorescent host is a material for forming a phosphorescent emitting layer containing a phosphorescent dopant, but does not mean that it cannot be used as a material for forming a fluorescent emitting layer. The same can be applied to the fluorescent host.

In one embodiment, it is preferred that the emitting layer comprise the compound represented by formula (Ia) or (Ib) according to the present invention (hereinafter, these compounds may be referred to as the “compound (Ia) or (Ib)”). More preferably, it is contained as a dopant material. Further, it is preferred that the compound (Ia) or (Ib) be contained in the emitting layer as a fluorescent dopant. Even further, it is preferred that the compound (Ia) or (Ib) be contained in the emitting layer as a blue fluorescent dopant.

In one embodiment, no specific restrictions are imposed on the content of the compound (Ia) or (Ib) as the dopant material in the emitting layer. In respect of sufficient emission and concentration quenching, the content is preferably 0.5 to 70 mass %, more preferably 0.8 to 30 mass %, further preferably 1 to 30 mass %, still further preferably 1 to 20 mass %, and particularly preferably 1 to 10 mass %, further particularly preferably 1 to 5 mass %, even further particularly preferably 2 to 4 mass %, related to the mass of the emitting layer.

### Fluorescent Dopant

As a fluorescent dopant other than the compound (Ia) or (Ib), a fused polycyclic aromatic compound, a styrylamine compound, a fused ring amine compound, a boron-containing compound, a pyrrole compound, an indole compound, a carbazole compound can be given, for example. Among these, a fused ring amine compound, a boron-containing compound, carbazole compound is preferable.

As the fused ring amine compound, a diaminopyrene compound, a diaminochrysene compound, a diaminoanthracene compound, a diaminofluorene compound, a diaminofluorene compound with which one or more benzofuro skeletons are fused, or the like can be given.

As the boron-containing compound, a pyrromethene compound, a triphenylborane compound or the like can be given.

### Phosphorescent Dopant

As a phosphorescent dopant, a phosphorescent emitting heavy metal complex and a phosphorescent emitting rare earth metal complex can be given.

As the heavy metal complex, an iridium complex, an osmium complex, a platinum complex or the like can be given. The heavy metal complex is for example an ortho-metallated complex of a metal selected from iridium, osmium and platinum.

Examples of rare earth metal complexes include terbium complexes, europium complexes and the like. Specifically, tris(acetylacetonate)(monophenanthroline)terbium(III) (abbreviation: Tb(acac)₃(Phen)), tris(1,3-diphenyl-1,3-propandionate)(monophenanthroline)europium(III) (abbreviation: Eu(DBM)₃(Phen)), tris[1-(2-thenoyl)-3,3,3-trifluoroacetonate](monophenanthroline)europium(III) (abbreviation: Eu(TTA)₃(Phen)) or the like can be given. These rare earth metal complexes are preferable as phosphorescent dopants since rare earth metal ions emit light due to electronic transition between different multiplicity.

As a blue phosphorescent dopant, an iridium complex, an osmium complex, a platinum complex, or the like can be given, for example. Specifically, bis[2-(4′,6′-difluorophenyl)pyridinate-N,C2′]iridium(III) tetrakis(1-pyrazolyl)borate (abbreviation: FIr6), bis[2-(4′,6′-difluorophenyl) pyridinato-N,C2′]iridium(III) picolinate (abbreviation: Ir(CF₃ppy)₂(pic)), bis[2-(4′,6′-difluorophenyl)pyridinato-N,C2′]iridium(III) acetylacetonate (abbreviation: FIracac) or the like can be given.

As a green phosphorescent dopant, an iridium complex or the like can be given, for example. Specifically, tris(2-phenylpyridinato-N,C2′) iridium(III) (abbreviation: Ir(ppy)₃), bis(1,2-diphenyl-1H-benzimidazolato)iridium(III) acetylacetonate (abbreviation: Ir(pbi)₂(acac)), bis(benzo[h]quinolinato)iridium(III) acetylacetonate (abbreviation: Ir(bzq)₂(acac)) or the like can be given.

As a red phosphorescent dopant, an iridium complex, a platinum complex, a terbium complex, an europium complex or the like can be given. Specifically, bis[2-(2′-benzo[4,5-α]thienyl)pyridinato-N,C3′]iridium(III) acetylacetonate (abbreviation: Ir(btp)₂(acac)), bis(1-phenylisoquinolinato-N,C2′)iridium(III) acetylacetonate (abbreviation: Ir(piq)₂(acac)), (acetylacetonato)bis[2,3-bis(4-fluorophenyl)quinoxalinato]iridium(III) (abbreviation: Ir(Fdpq)₂(acac)), 2,3,7,8,12,13,17,18-octaethyl-21H,23H-porphyrin platinum(II) (abbreviation PtOEP) or the like can be given.

As mentioned above, the emitting layer preferably comprises at least one compound (Ia) or (Ib) as a dopant.

### Host Material

As host material, metal complexes such as aluminum complexes, beryllium complexes and zinc complexes; heterocyclic compounds such as indole compounds, pyridine compounds, pyrimidine compounds, triazine compounds, quinoline compounds, isoquinoline compounds, quinazoline compounds, dibenzofuran compounds, dibenzothiophene compounds, oxadiazole compounds, benzimidazole compounds, and phenanthroline compounds; fused polyaromatic hydrocarbon (PAH) compounds such as a naphthalene compound, a triphenylene compound, a carbazole compound, an anthracene compound, a phenanthrene compound, a pyrene compound, a chrysene compound, a naphthacene compound, and a fluoranthene compound; and aromatic amine compound such as triarylamine compounds and fused polycyclic aromatic amine compounds can be given, for example. Plural types of host materials can be used in combination.

As a fluorescent host, a compound having a higher singlet energy level than a fluorescent dopant is preferable. For example, a heterocyclic compound, a fused aromatic compound or the like can be given. As a fused aromatic compound, an anthracene compound, a pyrene compound, a chrysene compound, a naphthacene compound or the like are preferable. An anthracene compound is preferentially used as blue fluorescent host.

As a phosphorescent host, a compound having a higher triplet energy level as compared with a phosphorescent dopant is preferable. For example, a metal complex, a heterocyclic compound, a fused aromatic compound or the like can be given. Among these, an indole compound, a carbazole compound, a pyridine compound, a pyrimidine compound, a triazine compound, a quinolone compound, an isoquinoline compound, a quinazoline compound, a dibenzofuran compound, a dibenzothiophene compound, a naphthalene compound, a triphenylene compound, a phenanthrene compound, a fluoranthene compound or the like can be given.

Preferred host materials are substituted or unsubstituted polyaromatic hydrocarbon (PAH) compounds, substituted or unsubstituted polyheteroaromatic compounds, substituted or unsubstituted anthracene compounds, or substituted or unsubstituted pyrene compounds, preferably substituted or unsubstituted anthracene compounds or substituted or unsubstituted pyrene compounds, more preferably substituted or unsubstituted anthracene compounds, most preferably anthracene compounds represented by formula (10) below.

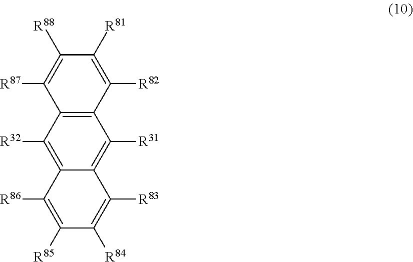

In the formula (10), Ar³¹ and Ar³² each independently represent a substituted or unsubstituted aryl group having 6 to 50 ring carbon atoms or a heterocyclic group having 5 to 50 ring atoms.

R⁸¹ to R⁸⁸ each independently represent a hydrogen atom, a substituted or unsubstituted aryl group having 6 to 50 ring carbon atoms, a substituted or unsubstituted heterocyclic group having 5 to 50 ring atoms, a substituted or unsubstituted alkyl group having 1 to 50 carbon atoms, a substituted or unsubstituted alkoxy group having 1 to 50 carbon atoms, a substituted or unsubstituted aralkyl group having 7 to 50 carbon atoms, a substituted or unsubstituted aryloxy group having 6 to 50 ring carbon atoms, a substituted or unsubstituted arylthio group having 6 to 50 ring carbon atoms, a substituted or unsubstituted alkoxycarbonyl group having 2 to 50 carbon atoms, a substituted or unsubstituted silyl group, a carboxyl group, a halogen atom, a cyano group, a nitro group or a hydroxyl group.

In formula (10):

The aryl group having 6 to 50 ring carbon atoms is preferably an aryl group having 6 to 40 ring carbon atoms, more preferably an aryl group having 6 to 30 ring carbon atoms.

The heterocyclic group having 5 to 50 ring atoms is preferably a heterocyclic group having 5 to 40 ring atoms, more preferably a heterocyclic group having 5 to 30 ring atoms. More preferably, the heterocyclic group is a substituted or unsubstituted heteroaryl group having 5 to 30 ring atoms. Suitable substituted or unsubstituted heteroaryl groups are mentioned above.

The alkyl group having 1 to 50 carbon atoms is preferably an alkyl group having 1 to 30 carbon atoms, more preferably an alkyl group having 1 to 10 carbon atoms, further preferably an alkyl group having 1 to 5 carbon atoms.

The alkoxy group having 1 to 50 carbon atoms is preferably an alkoxy group having 1 to 30 carbon atoms, more preferably an alkoxy group having 1 to 10 carbon atoms, further preferably an alkoxy group having 1 to 5 carbon atoms.

The aralkyl group having 7 to 50 carbon atoms is preferably an aralkyl group having 7 to 30 carbon atoms, more preferably an aralkyl group having 7 to 20 carbon atoms.

The aryloxy group having 6 to 50 ring carbon atoms is preferably an aryloxy group having 6 to 40 ring carbon atoms, more preferably an aryloxy group having 6 to 30 ring carbon atoms.

The arylthio group having 6 to 50 ring carbon atoms is preferably an arylthio group having 6 to 40 ring carbon atoms, more preferably an arylthio group having 6 to 30 ring carbon atoms.

The alkoxycarbonyl group having 2 to 50 carbon atoms is preferably an alkoxycarbonyl group having 2 to 30 carbon atoms, more preferably an alkoxycarbonyl group having 2 to 10 carbon atoms, further preferably an alkoxycarbonyl group having 2 to 5 carbon atoms.

Examples of the halogen atom are a fluorine atom, a chlorine atom and a bromine atom.

Ar³¹ and Ar³² are preferably a substituted or unsubstituted aryl group having 6 to 50 ring carbon atoms.

### Electron-Transporting Zone, Electron-Transporting Layer, Electron-Injecting Layer, Hole-Blocking Layer

The electron-transporting zone is an organic layer or a plural of organic layers that is formed between the emitting layer and the cathode and has a function of transporting electrons from the cathode to the emitting layer. The electron-transporting zone therefore comprises at least one electron-transporting layer comprising an electron-transporting material. When the electron-transporting zone is formed of plural layers, an organic layer or an inorganic layer that is nearer to the cathode is often defined as the electron-injecting layer (see for example FIG. 1, wherein an electron-injecting layer 9, an electron-transporting layer 8 and preferably a hole-blocking layer 7 form an electron-transporting zone 11). The electron-injecting layer has a function of injecting electrons from the cathode efficiently to the organic layer unit. Preferred electron-injecting materials are alkali metals, alkali metal compounds, alkali metal complexes, alkaline earth metal complexes, rare earth metals, or rare earth metal complexes.

According to one embodiment, it is therefore preferred that the electron-transporting zone comprises in addition to the electron-transporting layer one or more layer(s) like an electron-injecting layer to enhance efficiency and lifetime of the device, a hole-blocking layer or an exciton/triplet-blocking layer (layer 7 in FIG. 1).

In one embodiment of the present invention, the compound of the formula (Ia) or (Ib) is present in the electron-transporting zone, as an electron-transporting material, an electron-injecting material, a hole-blocking material, an exciton-blocking material and/or a triplet-blocking material.

According to one embodiment, it is preferred that an electron-donating dopant be contained in the interfacial region between the cathode and the emitting unit. Due to such a configuration, the organic EL device can have an increased luminance or a long life. Here, the electron-donating dopant means one having a metal with a work function of 3.8 eV or less. As specific examples thereof, at least one selected from an alkali metal, an alkali metal complex, an alkali metal compound, an alkaline earth metal, an alkaline earth metal complex, an alkaline earth metal compound, a rare earth metal, a rare earth metal complex, and a rare earth metal compound or the like can be mentioned.

As the alkali metal, Li (work function: 2.9 eV), Na (work function: 2.36 eV), K (work function: 2.28 eV), Rb (work function: 2.16 eV), Cs (work function: 1.95 eV) and the like can be given. One having a work function of 2.9 eV or less is particularly preferable. Among them, K, Rb and Cs are preferable. Rb or Cs is further preferable. Cs is most preferable. As the alkaline earth metal, Ca (work function: 2.9 eV), Sr (work function: 2.0 eV to 2.5 eV), Ba (work function: 2.52 eV), Mg (work function: 3.68 eV) and the like can be given. One having a work function of 2.9 eV or less is particularly preferable. As the rare-earth metal, Sc, Y, Ce, Tb, Yb and the like can be given. One having a work function of 2.9 eV or less is particularly preferable.

Examples of the alkali metal compound include an alkali chalcogenide such as Li₂O, Na₂O, Cs₂O, K₂O, Na₂S or Na₂Se, and an alkali halide such as LiF, NaF, CsF, KF, LiCl, KCl and NaCl. Among them, LiF, Li₂O and NaF are preferable. Examples of the alkaline earth metal compound include BaO, SrO, CaO, BeO, BaS, CaSe and mixtures thereof such as Ba, Sr₁₋ₓO (0<x<1) and BaₓCa₁₋ₓO (0<x<1). Alkaline earth metal halides are for example fluorides such as CaF₂, BaF₂, SrF₂, MgF₂ and BeF₂. Among them, BaO, SrO and CaO are preferable. Examples of the rare earth metal compounds include one or more oxides, nitrides, oxidized nitrides or halides, especially fluorides, containing at least one element selected from Yb, Sc, Y, Ce, Gd, Tb and the like, for example YbF₃, ScF₃, ScO₃, Y₂O₃, Ce₂O₃, GdF₃ and TbF₃. Among these, YbF₃, ScF₃ and TbF₃ are preferable. Further suitable dopants are one or more oxides, nitrides and oxidized nitrides of Al, Ga, In, Cd, Si, Ta, Sb and Zn and nitrides and oxidized nitrides of Ba, Ca, Sr, Yb, Li, Na and Mg.

The alkali metal complexes, the alkaline earth metal complexes and the rare earth metal complexes are not particularly limited as long as they contain, as a metal ion, at least one of alkali metal ions, alkaline earth metal ions, and rare earth metal ions. Meanwhile, preferred examples of the ligand include, but are not limited to, quinolinol, benzoquinolinol, acridinol, phenanthridinol, hydroxyphenyloxazole, hydroxyphenylthiazole, hydroxydiaryloxadiazole, hydroxydiarylthiadiazole, hydroxyphenylpyridine, hydroxyphenylbenzimidazole, hydroxybenzotriazole, hydroxyfluborane, bipyridyl, phenanthroline, phthalocyanine, porphyrin, cyclopentadiene, β-diketones, and azomethines.

Regarding the addition form of the electron-donating dopant, it is preferred that the electron-donating dopant be formed in a shape of a layer or an island in the interfacial region. A preferred method for the formation is a method in which an organic compound (a light emitting material or an electron-injecting material) for forming the interfacial region is deposited simultaneously with deposition of the electron-donating dopant by a resistant heating deposition method, thereby dispersing the electron-donating dopant in the organic compound.

In a case where the electron-donating dopant is formed into the shape of a layer, the light-emitting material or electron-injecting material which serves as an organic layer in the interface is formed into the shape of a layer. After that, a reductive dopant is solely deposited by the resistant heating deposition method to form a layer preferably having a thickness of from 0.1 nm to 15 nm.

In a case where the electron-donating dopant is formed into the shape of an island, the emitting material or the electron-injecting material which serves as an organic layer in the interface is formed into the shape of an island. After that, the electron-donating dopant is solely deposited by the resistant heating deposition method to form an island preferably having a thickness of from 0.05 nm to 1 nm.

As the electron-transporting material used in the electron-transporting layer other than a compound of the formula (Ia) or (Ib), an aromatic heterocyclic compound having one or more hetero atoms in the molecule may preferably be used. In particular, a nitrogen containing heterocyclic compound is preferable.

According to one embodiment, it is preferable that the electron-transporting layer comprises a nitrogen containing heterocyclic metal chelate.

According to another embodiment, it is preferable that the electron-transporting layer comprises a substituted or unsubstituted nitrogen containing heterocyclic compound. Specific examples of preferred heterocyclic compounds for the electron-transporting layer are, 6-membered azine compounds; such as pyridine compounds, pyrimidine compounds, triazine compounds, and pyrazine compounds, preferably pyrimidine compounds or triazine compounds; 6-membered fused azine compounds, such as quinolone compounds, isoquinoline compounds, quinoxaline compounds, quinazoline compounds, phenanthroline compounds, benzoquinoline compounds, benzoisoquinoline compounds, and dibenzoquinoxaline compounds, preferably quinolone compounds, isoquinoline compounds, or phenanthroline compounds; 5-membered heterocyclic compounds, such as imidazole compounds, oxazole compounds, oxadiazole compounds, triazole compounds, thiazole compounds, and thiadiazole compounds; fused imidazole compounds, such as benzimidazole compounds, imidazopyridine compounds, naphthoimidazole compounds, benzimidazophenanthridine compounds, and benzimidzobenzimidazole compounds, preferably benzimidazole compounds, imidazopyridine compounds or benzimidazophenanthridine compounds.

According to another embodiment, it is preferable the electron-transporting layer comprises a phosphine oxide compound represented as Arₚ₁Arₚ₂Arₚ₃P═O.

Arₚ₁ to Arₚ₃ are the substituents of phosphor atom and each independently represent substituted or unsubstituted above mentioned aryl group or substituted or unsubstituted above mentioned heterocyclic group.

According to another embodiment, it is preferable that the electron-transporting layer comprises aromatic hydrocarbon compounds. Specific examples of preferred aromatic hydrocarbon compounds for the electron-transporting layer are oligo-phenylene compounds, naphthalene compounds, fluorene compounds, fluoranthenyl group, anthracene compounds, phenanthrene compounds, pyrene compounds, triphenylene compounds, benzanthracene compounds, chrysene compounds, benzphenanthrene compounds, naphthacene compounds, and benzochrysene compounds, preferably anthracene compounds, pyrene compounds, and fluoranthene compounds.

A hole-blocking layer may be provided adjacent to the emitting layer and has a function of preventing leakage of holes from the emitting layer to the electron-transporting layer. In order to improve hole-blocking capability, a material having a deep HOMO level is preferably used.

In a preferred embodiment, the organic electroluminescence device according to the present invention comprises an electron-transporting zone, wherein the electron-transporting zone further comprises at least one of an electron-donating dopant and preferably an organic metal complex. Suitable dopants are mentioned above.

More preferably, the at least one of an electron-donating dopant and an organic metal complex is at least one selected from the group consisting of an alkali metal, an alkali metal compound, an alkali metal complex, an alkaline earth metal, an alkaline earth metal compound, an alkaline earth metal complex, a rare earth metal, a rare earth metal compound, and a rare earth metal complex.

### Cathode

For the cathode, a metal, an alloy, an electrically conductive compound, and a mixture thereof, each having a small work function (specifically, a work function of 3.8 eV or less) are preferably used. Specific examples of a material for the cathode include an alkali metal such as lithium and cesium; an alkaline earth metal such as magnesium, calcium, and strontium; an alloy containing these metals (for example, magnesium-silver, aluminum-lithium); a rare earth metal such as europium and ytterbium; and an alloy containing a rare earth metal.

The cathode is usually formed by a vacuum vapor deposition or a sputtering method. Further, in the case of using a silver paste or the like, a coating method, an inkjet method, or the like can be employed.

Moreover, when the electron-injecting layer is provided, various electrically conductive materials such as aluminum, silver, ITO, graphene, indium oxide-tin oxide containing silicon or silicon oxide, selected independently from the work function, can be used to form a cathode.

These electrically conductive materials are made into films using a sputtering method, an inkjet method, a spin coating method, or the like.

### Insulating Layer

In the organic EL device, pixel defects based on leakage or a short circuit are easily generated since an electric field is applied to a thin film. In order to prevent this, it is preferred to insert an insulating thin layer between a pair of electrodes. Examples of materials used in the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture thereof may be used in the insulating layer, and a laminate of a plurality of layers that include these materials can be also used for the insulating layer.

### Spacing Layer

A spacing layer is a layer for example provided between a fluorescent emitting layer and a phosphorescent emitting layer when a fluorescent emitting layer and a phosphorescent emitting layer are stacked in order to prevent diffusion of excitons generated in the phosphorescent emitting layer to the fluorescent emitting layer or in order to adjust the carrier balance. Further, the spacing layer can be provided between the plural phosphorescent emitting layers.

Since the spacing layer is provided between the emitting layers, the material used for the spacing layer is preferably a material having both electron-transporting capability and hole-transporting capability. In order to prevent diffusion of the triplet energy in adjacent phosphorescent emitting layers, it is preferred that the spacing layer have a triplet energy of 2.6 eV or more. As the material used for the spacing layer, the same materials as those used in the above-mentioned hole-transporting layer can be given.

### Triplet-Blocking Layer

A triplet-blocking layer (exciton-blocking layer) may be provided adjacent to the emitting layer.

The triplet-blocking layer has a function of preventing triplet excitons generated in the emitting layer from diffusing into neighboring layers to trap the triplet excitons within the emitting layer, thereby suppressing energy deactivation of the triplet excitons on molecules other than the emitting dopant in the electron-transporting layer.

When the triplet-blocking layer is provided in a phosphorescent device, triplet energy of a phosphorescent dopant in the emitting layer is denoted as E^{T}_{d} and triplet energy of a compound used as the triplet-blocking layer is denoted as E^{T}_{TB}. In an energy relationship of E^{T}_{d}<E^{T}_{TB}, triplet excitons of the phosphorescent dopant are trapped (cannot be transferred to another molecule) to leave no alternative route for energy deactivation other than emission on the dopant, so that highly, efficient emission can be expected. However, when an energy gap (ΔE^{T}=E^{T}_{TB}−E^{T}_{d}) is small even though the relationship of E^{T}_{d}<E^{T}_{TB} is satisfied, under actual environments for driving a device (i.e., at around the room temperature), it is considered that triplet excitons can be transferred to another molecule irrespective of the energy gap ΔE^{T} by absorbing heat energy around the device. Particularly, since the excitons of the phosphorescent device have longer lifetime than those of a fluorescent device, influence by heat absorption during transfer of the excitons is more likely to be given on the phosphorescent device relative to the fluorescent device. A larger energy gap ΔE^{T} relative to heat energy at the room temperature is preferable, more preferably 0.1 eV or more, further preferable at 0.2 eV or more. On the other hand, in the fluorescent device, the organic-EL-device material according to the exemplary embodiment is usable as the triplet-blocking layer in the TTF device structure described in International Publication WO2010/134350A1.

### Method for Forming a Layer

The method for forming each layer of the organic EL device of the invention is not particularly limited unless otherwise specified. A known film-forming method such as a dry film-forming method, a wet film-forming method or the like can be used. Specific examples of the dry film-forming method include a vacuum deposition method, a sputtering method, a plasma method, an ion plating method, and the like. Specific examples of the wet film-forming method include various coating methods such as a spin coating method, a dipping method, a flow coating method, an inkjet method, and the like.

### Film Thickness

The film thickness of each layer of the organic EL device of the invention is not particularly limited unless otherwise specified. If the film thickness is too small, defects such as pinholes are likely to occur to make it difficult to obtain a sufficient luminance. If the film thickness is too large, a high driving voltage is required to be applied, leading to a lowering in efficiency. In this respect, the film thickness is preferably 5 nm to 10 μm, and more preferably 10 nm to 0.2 μm.

### Electronic Apparatus (Electronic Equipment)

The present invention further relates to an electronic equipment (electronic apparatus) comprising the organic electroluminescence device according to the present application. Examples of the electronic apparatus include display parts such as an organic EL panel module; display devices of television sets, mobile phones, smart phones, and personal computer, and the like; and emitting devices of a lighting device and a vehicle lighting device.

It should be noted that the invention is not limited to the above exemplary embodiments but may include any modification and improvement as long as such modification and improvement are compatible with the invention.

### EXAMPLES

The following examples are included for illustrative purposes only and do not limit the scope of the claims. Unless otherwise stated, all parts and percentages are by weight.

Compounds used in organic EL devices of Application Examples 1 to 15

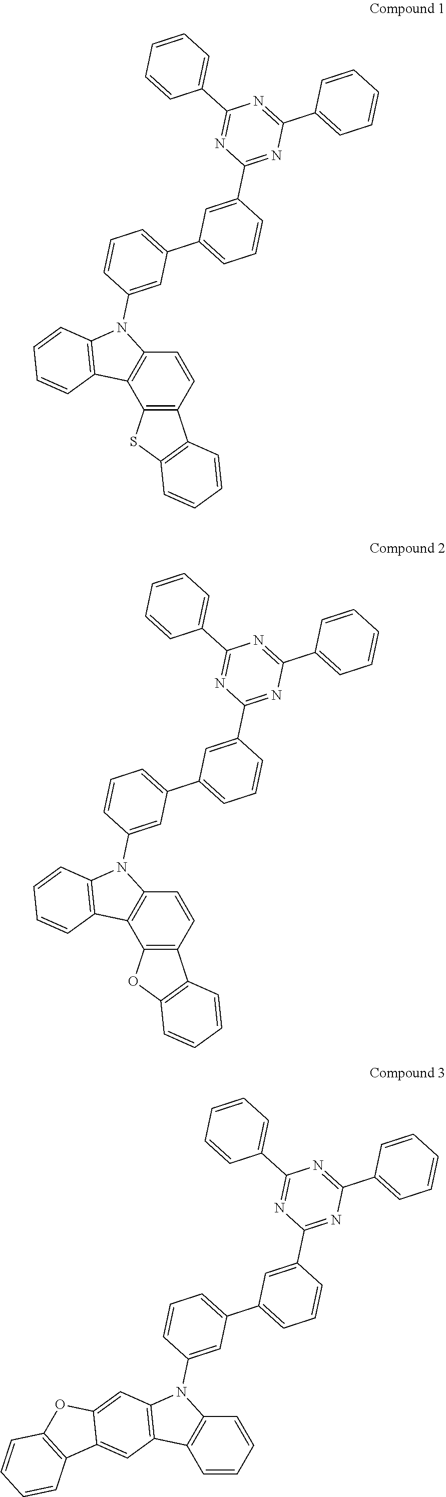 [SMILES: c1ccc(-c2nc(-c3ccccc3)nc(-c3cccc(-c4cccc(-n5c6ccccc6c6c7sc8ccccc8c7ccc65)c4)c3)n2)cc1 ; c1ccc(-c2nc(-c3ccccc3)nc(-c3cccc(-c4cccc(-n5c6ccccc6c6c7oc8ccccc8c7ccc65)c4)c3)n2)cc1 ; c1ccc(-c2nc(-c3ccccc3)nc(-c3cccc(-c4cccc(-n5c6ccccc6c6cc7c(cc65)oc5ccccc57)c4)c3)n2)cc1]

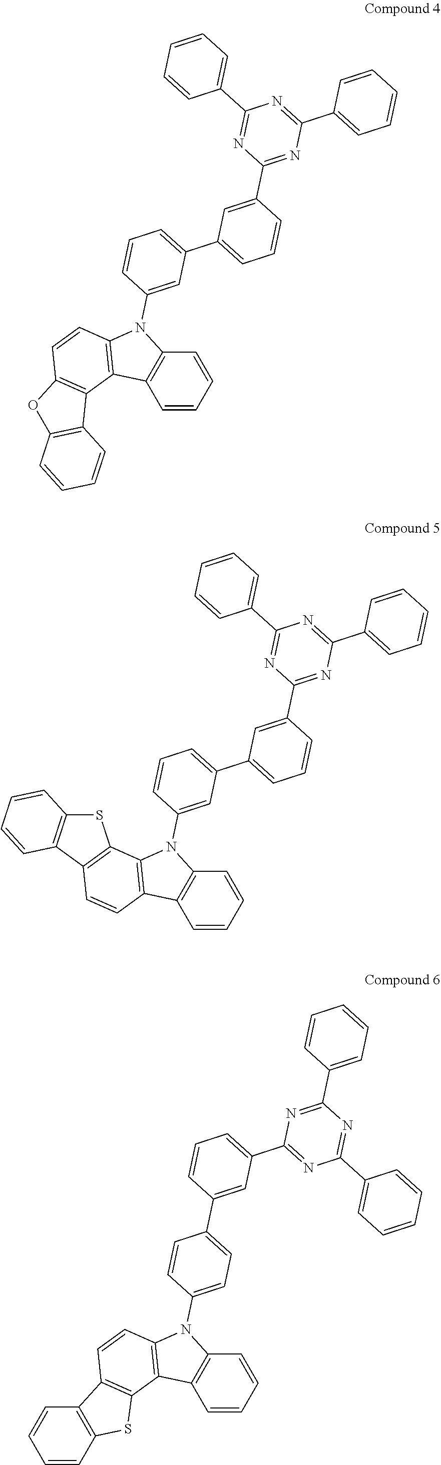 [SMILES: c1ccc(-c2nc(-c3ccccc3)nc(-c3cccc(-c4cccc(-n5c6ccccc6c6c7c(ccc65)oc5ccccc57)c4)c3)n2)cc1 ; c1ccc(-c2nc(-c3ccccc3)nc(-c3cccc(-c4cccc(-n5c6ccccc6c6ccc7c8ccccc8sc7c65)c4)c3)n2)cc1 ; c1ccc(-c2nc(-c3ccccc3)nc(-c3cccc(-c4ccc(-n5c6ccccc6c6c7sc8ccccc8c7ccc65)cc4)c3)n2)cc1]

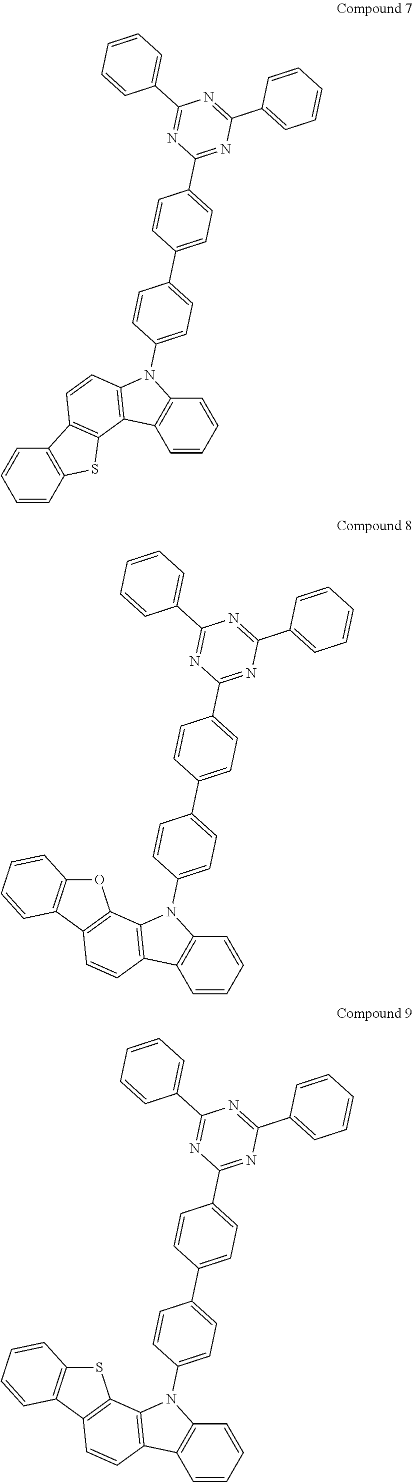 [SMILES: c1ccc(-c2nc(-c3ccccc3)nc(-c3ccc(-c4ccc(-n5c6ccccc6c6c7sc8ccccc8c7ccc65)cc4)cc3)n2)cc1 ; c1ccc(-c2nc(-c3ccccc3)nc(-c3ccc(-c4ccc(-n5c6ccccc6c6ccc7c8ccccc8oc7c65)cc4)cc3)n2)cc1 ; c1ccc(-c2nc(-c3ccccc3)nc(-c3ccc(-c4ccc(-n5c6ccccc6c6ccc7c8ccccc8sc7c65)cc4)cc3)n2)cc1]

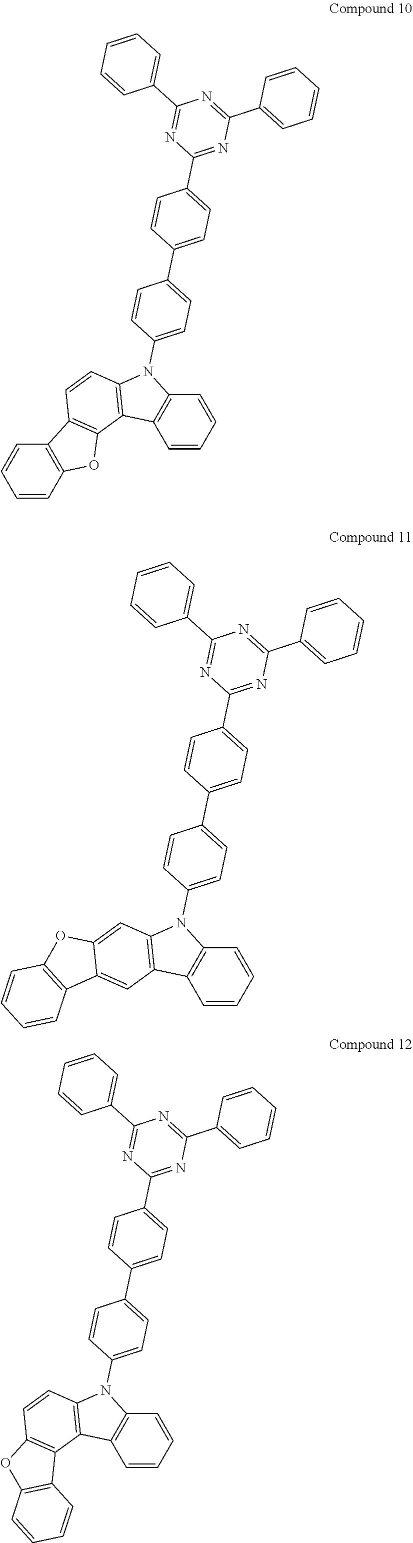

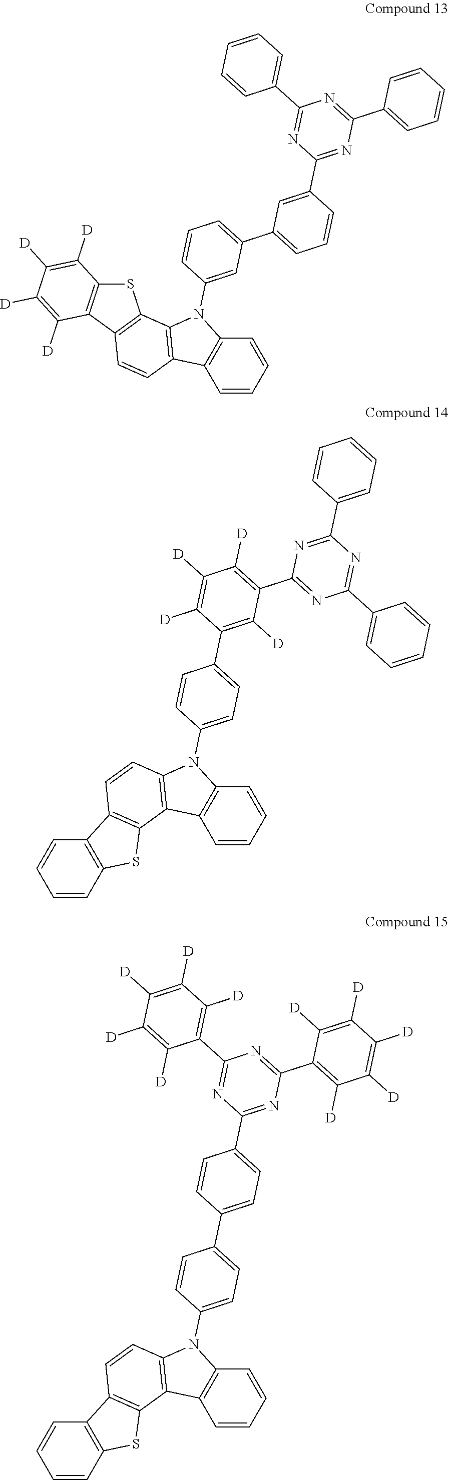 [SMILES: [2H]c1c([2H])c([2H])c2c(sc3c2ccc2c4ccccc4n(-c4cccc(-c5cccc(-c6nc(-c7ccccc7)nc(-c7ccccc7)n6)c5)c4)c23)c1[2H] ; [2H]c1c([2H])c(-c2ccc(-n3c4ccccc4c4c5sc6ccccc6c5ccc43)cc2)c([2H])c(-c2nc(-c3ccccc3)nc(-c3ccccc3)n2)c1[2H] ; [2H]c1c([2H])c([2H])c(-c2nc(-c3ccc(-c4ccc(-n5c6ccccc6c6c7sc8ccccc8c7ccc65)cc4)cc3)nc(-c3c([2H])c([2H])c([2H])c([2H])c3[2H])n2)c([2H])c1[2H]]

Compounds used in organic EL devices of Comparative Application Examples 1 to 6

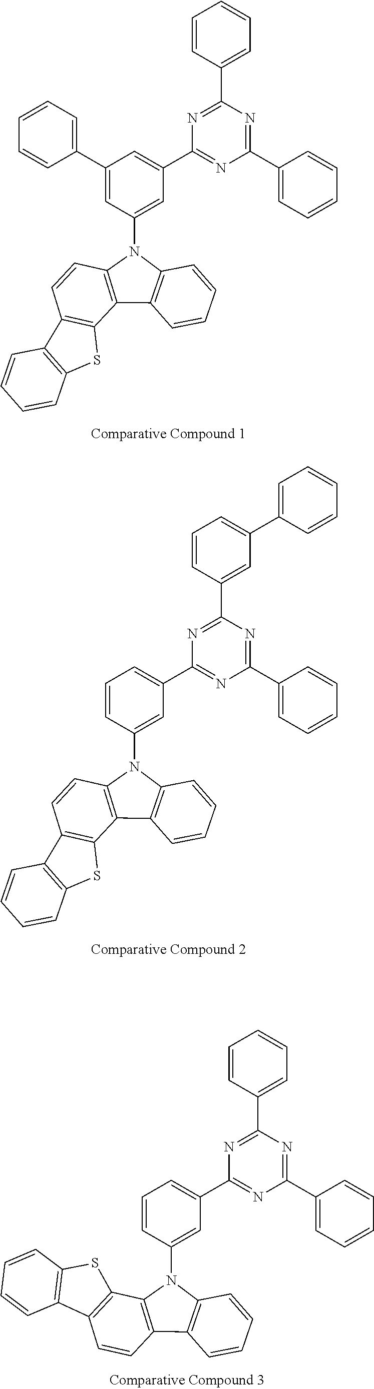 [SMILES: c1ccc(-c2cc(-c3nc(-c4ccccc4)nc(-c4ccccc4)n3)cc(-n3c4ccccc4c4c5sc6ccccc6c5ccc43)c2)cc1 ; c1ccc(-c2cccc(-c3nc(-c4ccccc4)nc(-c4cccc(-n5c6ccccc6c6c7sc8ccccc8c7ccc65)c4)n3)c2)cc1 ; c1ccc(-c2nc(-c3ccccc3)nc(-c3cccc(-n4c5ccccc5c5ccc6c7ccccc7sc6c54)c3)n2)cc1]

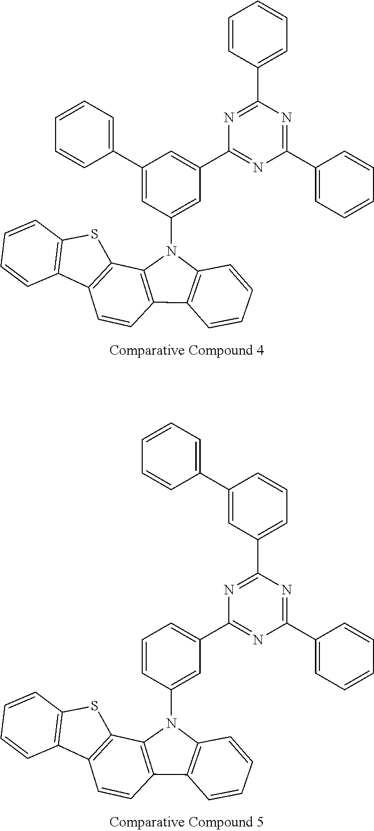 [SMILES: c1ccc(-c2cc(-c3nc(-c4ccccc4)nc(-c4ccccc4)n3)cc(-n3c4ccccc4c4ccc5c6ccccc6sc5c43)c2)cc1 ; c1ccc(-c2cccc(-c3nc(-c4ccccc4)nc(-c4cccc(-n5c6ccccc6c6ccc7c8ccccc8sc7c65)c4)n3)c2)cc1]

Other compounds used in organic EL devices of Application Examples 1 to 6 and Comparative Application Examples 1 to 5

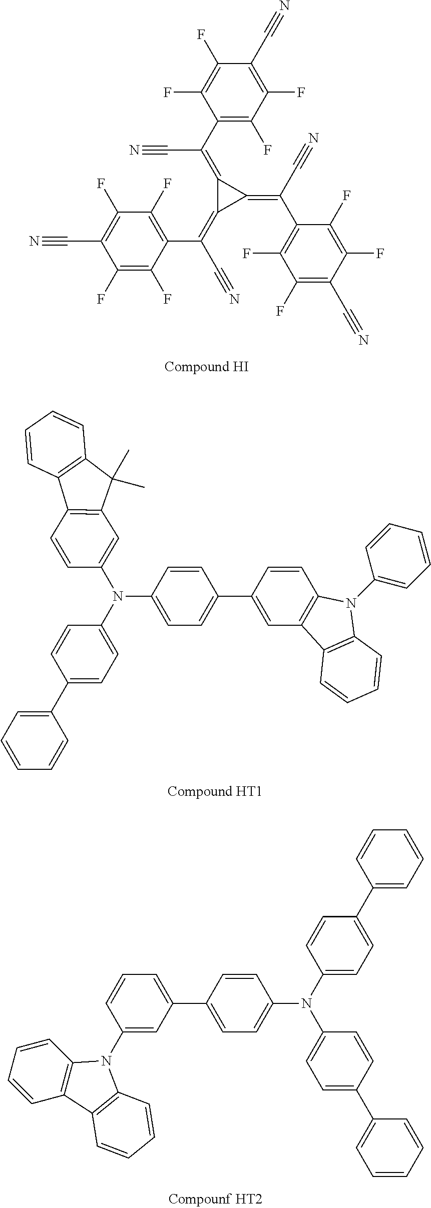 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc(-c4cccc(-n5c6ccccc6c6ccccc65)c4)cc3)cc2)cc1]

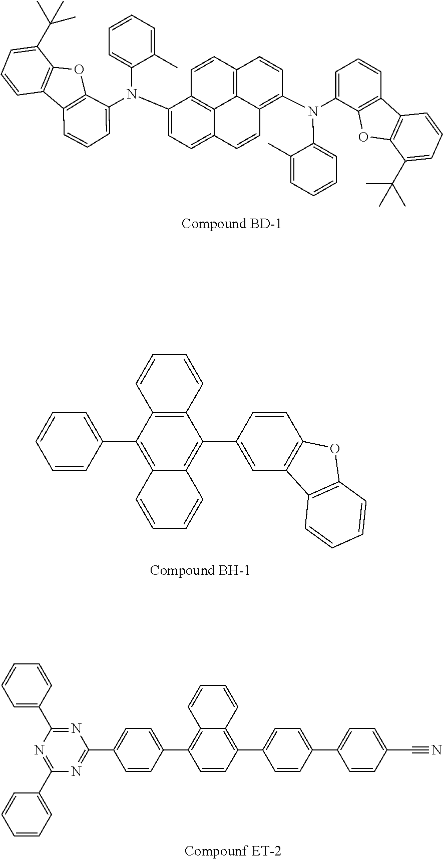 [SMILES: Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12 ; c1ccc(-c2c3ccccc3c(-c3ccc4oc5ccccc5c4c3)c3ccccc23)cc1 ; N#Cc1ccc(-c2ccc(-c3ccc(-c4ccc(-c5nc(-c6ccccc6)nc(-c6ccccc6)n5)cc4)c4ccccc34)cc2)cc1]

Other compounds used in organic EL devices of Application Examples 7 to 15 and Comparative Application Example 6

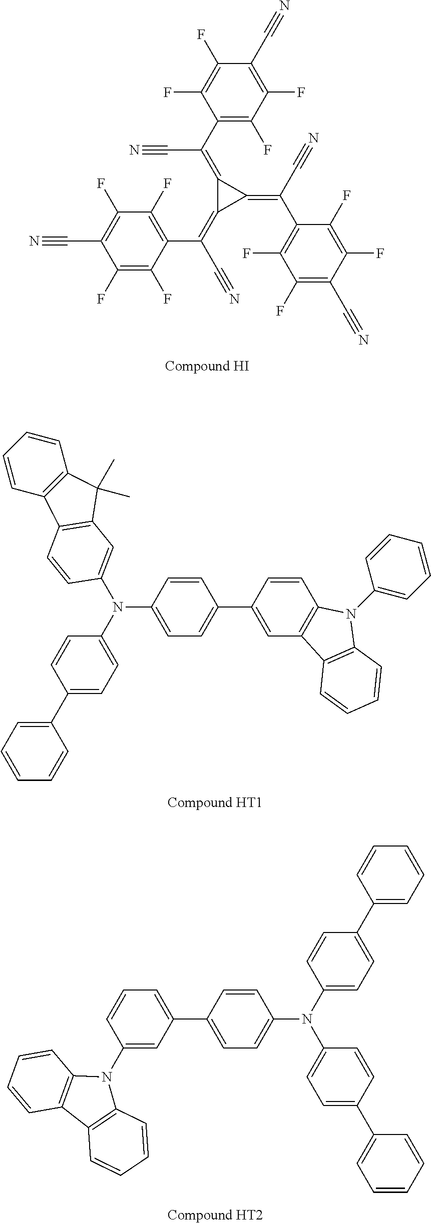 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc(-c4cccc(-n5c6ccccc6c6ccccc65)c4)cc3)cc2)cc1]

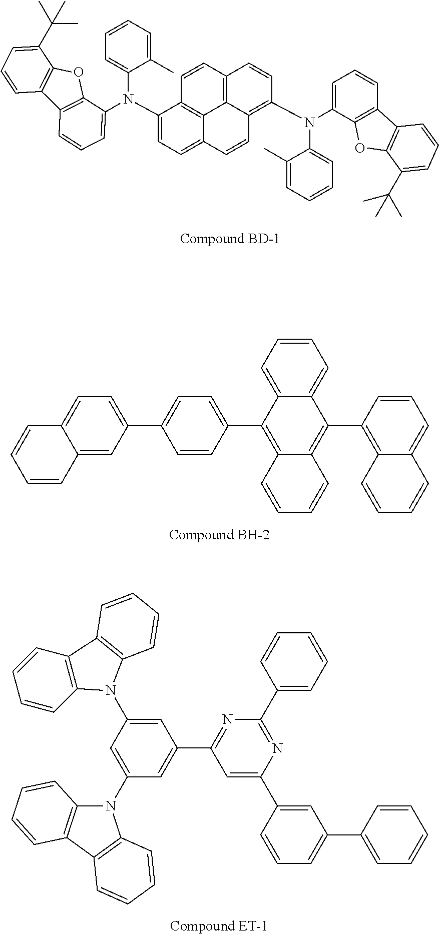

### Application Example 1

A glass substrate with 130 nm-thick indium-tin-oxide (ITO) transparent electrode (manufactured by Geomatec Co., Ltd.) used as an anode was first treated with N₂ plasma for 100 sec. This treatment also improved the hole injection properties of the ITO.

The cleaned substrate was mounted on a substrate holder and loaded into a vacuum chamber.

Thereafter, the organic materials specified below were applied by vapour deposition to the ITO substrate at a rate of approx. 0.2-1 Å/sec at about 10⁻⁶-10⁻⁸ mbar.

As a hole injection layer, 10 nm-thick mixture of 97% by weight of Compound HT1 and 3% by weight of Compound HI were applied.

Then 80 nm-thick of Compound HT1 and 5 nm of Compound HT2 were applied as hole transporting layer 1 and hole transporting layer 2, respectively.

Subsequently, a mixture of 4% by weight of an emitter Compound BD-1 and 96% by weight of host Compound BH-1 were applied to form a 25 nm-thick fluorescent-emitting layer.

On the emitting layer, 5 nm-thick Compound 1 was applied as an hole-blocking layer and 20 nm of mixture of 50% by weight of Compound E^{T}-2 and 50% by weight of lithiumquinolate (Liq) as electron-transporting layer.

Finally, 1 nm-thick Yb was deposited as an electron injection layer and 80 nm-thick Al was then deposited as a cathode to complete the device.

The device was sealed with a glass lid and a getter in an inert nitrogen atmosphere with less than 1 ppm of water and oxygen.

The layer structure of the device was:

ITO (130)/HT1:HI=97:3 (10)/HT1 (80)/HT2 (5)/BD-1:BH-1=4:96 (25)/Compound 1 (5)/E^{T}-2:Liq=50:50 (20)/Yb (1)/Al (80).

To characterize the OLED, electroluminescence spectra were recorded at various currents and voltages. In addition, the current-voltage characteristics were measured in combination with the luminance to determine external quantum efficiency (EQE). Driving voltage (Voltage) was given at a current density of 10 mA/cm². The device results are shown in Table 1.

### Application Example 2

Application Example 1 was repeated except for using Compound 2 instead of Compound 1. The device results are shown in Table 1.

### Application Example 3

Application Example 1 was repeated except for using Compound 3 instead of Compound 1. The device results are shown in Table 1.

### Application Example 4

Application Example 1 was repeated except for using Compound 4 instead of Compound 1. The device results are shown in Table 1.

### Application Example 5

Application Example 1 was repeated except for using Compound 5 instead of Compound 1. The device results are shown in Table 1.

### Application Example 6

Application Example 1 was repeated except for using Compound 13 instead of Compound 1. The device results are shown in Table 1.

### Comparative Application Example 1

Application Example 1 was repeated except for using Comparative Compound 1 instead of the Compound 1. The device results are shown in Table 1.

### Comparative Application Example 2

Application Example 1 was repeated except for using Comparative Compound 2 instead of the Compound 1. The device results are shown in Table 1.

### Comparative Application Example 3

Application Example 1 was repeated except for using Comparative Compound 3 instead of the Compound 1. The device results are shown in Table 1.

### Comparative Application Example 4

Application Example 1 was repeated except for using Comparative Compound 4 instead of the Compound 1. The device results are shown in Table 1.

### Comparative Application Example 5

Application Example 1 was repeated except for using Comparative Compound 4 instead of the Compound 1. The device results are shown in Table 1.

TABLE 1

| | Hole-blocking layer | | |
|---|---|---|---|
| Appl. Ex. | compound | Voltage, V | EQE, % |
| Appl. Ex. 1 | Compound 1 | 3.70 | 7.76 |
| Appl. Ex. 2 | Compound 2 | 3.82 | 8.82 |
| Appl. Ex. 3 | Compound 3 | 3.96 | 8.65 |
| Appl. Ex. 4 | Compound 4 | 3.93 | 8.74 |
| Appl. Ex. 5 | Compound 5 | 3.77 | 8.31 |
| Appl. Ex. 6 | Compound 13 | 3.79 | 8.30 |
| Comp. Appl. Ex. 1 | Comparative | 4.32 | 5.44 |
| | compound 1 | | |
| Comp. Appl. Ex. 2 | Comparative | 4.07 | 6.27 |
| | compound 2 | | |
| Comp. Appl. Ex. 3 | Comparative | 3.90 | 7.39 |
| | compound 3 | | |
| Comp. Appl. Ex. 4 | Comparative | 3.99 | 6.99 |
| | compound 4 | | |
| Comp. Appl. Ex. 5 | Comparative | 4.02 | 7.00 |
| | compound 5 | | |

These results demonstrated that the Inventive Compounds gave low voltage and better EQE than the Comparative Compounds when used as materials in the hole-blocking layer in OLED devices.

### Application Example 7

Application Example 1 was repeated except for using: Compound BH-2 instead of Compound BH-1 as a host in fluorescent-emitting layer; Compound E^{T}-1 instead of Compound 1 in the hole-blocking layer; and Compound 6 instead of Compound E^{T}-2 in the electron-transporting layer. Lifetime of OLED device was measured as a decay of the luminance at constant current density of 50 mA/cm² to 95% of its initial value. The device results are shown in Table 2.

### Application Example 8

Application Example 7 was repeated except for using Compound 7 instead of the Compound 6. The device results are shown in Table 2.

### Application Example 9

Application Example 7 was repeated except for using Compound 8 instead of the Compound 6. The device results are shown in Table 2.

### Application Example 10

Application Example 7 was repeated except for using Compound 9 instead of the Compound 6. The device results are shown in Table 2.

### Application Example 11

Application Example 7 was repeated except for using Compound 10 instead of the Compound 6. The device results are shown in Table 2.

### Application Example 12

Application Example 7 was repeated except for using Compound 11 instead of the Compound 6. The device results are shown in Table 2.

### Application Example 13

Application Example 7 was repeated except for using Compound 12 instead of the Compound 6. The device results are shown in Table 2.

### Application Example 14

Application Example 7 was repeated except for using Compound 14 instead of the Compound 6. The device results are shown in Table 2.

### Application Example 15

Application Example 7 was repeated except for using Compound 15 instead of the Compound 6. The device results are shown in Table 2.

### Comparative Application Example 6

Application Example 6 was repeated except for using Comparative Compound 1 instead of the Compound 6. The device results are shown in Table 2.

TABLE 2

| | Electron transporting | | |
|---|---|---|---|
| Appl. Ex. | layer compound | EQE, % | LT95, h |
| Appl. Ex. 7 | Compound 6 | 8.71 | 196 |
| Appl. Ex. 8 | Compound 7 | 9.03 | 207 |
| Appl. Ex. 9 | Compound 8 | 9.53 | 125 |
| Appl. Ex. 10 | Compound 9 | 9.14 | 65.5 |
| Appl. Ex. 11 | Compound 10 | 8.89 | 99.4 |
| Appl. Ex. 12 | Compound 11 | 9.35 | 155.0 |
| Appl. Ex. 13 | Compound 12 | 9.35 | 165.0 |
| Appl. Ex. 14 | Compound 14 | 8.72 | 197 |
| Appl. Ex. 15 | Compound 15 | 9.01 | 210 |
| Comp. Appl. | Comparative | 8.19 | 33 |
| Ex. 6 | compound 1 | | |

The results demonstrated that the inventive Compounds gave higher efficiency and longer lifetime than the Comparative Compound when used as materials in the electron-transporting layer in OLED devices.

### Compounds Synthesized in Preparation Examples 1 to 20

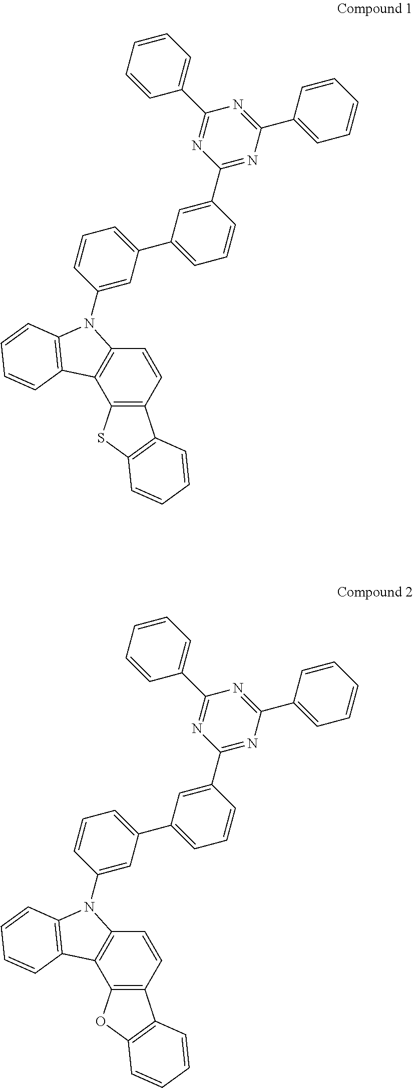 [SMILES: c1ccc(-c2nc(-c3ccccc3)nc(-c3cccc(-c4cccc(-n5c6ccccc6c6c7sc8ccccc8c7ccc65)c4)c3)n2)cc1 ; c1ccc(-c2nc(-c3ccccc3)nc(-c3cccc(-c4cccc(-n5c6ccccc6c6c7oc8ccccc8c7ccc65)c4)c3)n2)cc1]

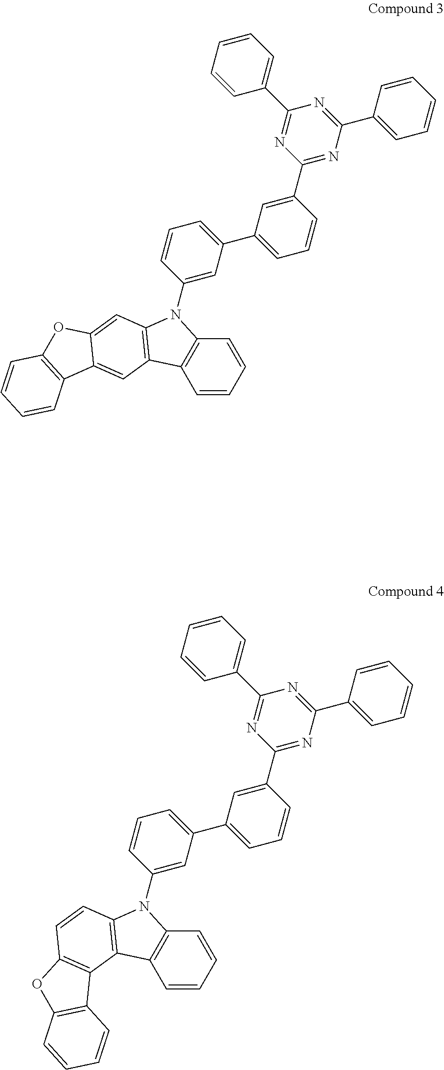 [SMILES: c1ccc(-c2nc(-c3ccccc3)nc(-c3cccc(-c4cccc(-n5c6ccccc6c6cc7c(cc65)oc5ccccc57)c4)c3)n2)cc1 ; c1ccc(-c2nc(-c3ccccc3)nc(-c3cccc(-c4cccc(-n5c6ccccc6c6c7c(ccc65)oc5ccccc57)c4)c3)n2)cc1]

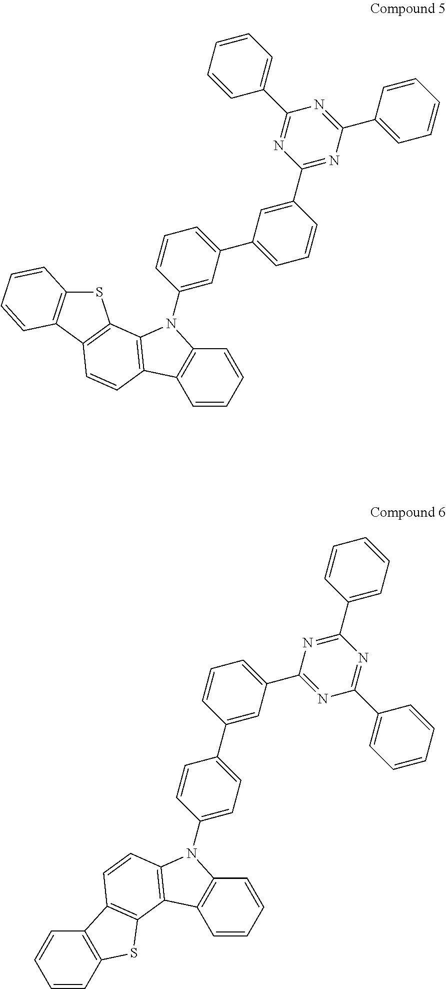 [SMILES: c1ccc(-c2nc(-c3ccccc3)nc(-c3cccc(-c4cccc(-n5c6ccccc6c6ccc7c8ccccc8sc7c65)c4)c3)n2)cc1 ; c1ccc(-c2nc(-c3ccccc3)nc(-c3cccc(-c4ccc(-n5c6ccccc6c6c7sc8ccccc8c7ccc65)cc4)c3)n2)cc1]

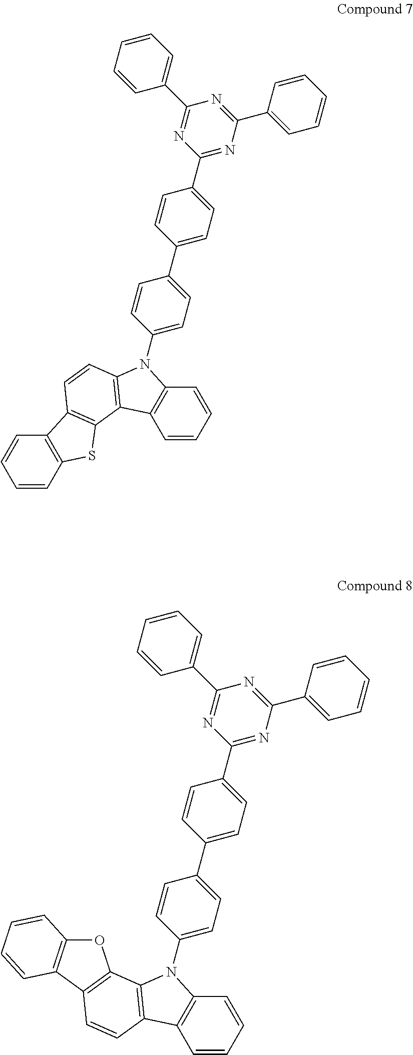 [SMILES: c1ccc(-c2nc(-c3ccccc3)nc(-c3ccc(-c4ccc(-n5c6ccccc6c6c7sc8ccccc8c7ccc65)cc4)cc3)n2)cc1 ; c1ccc(-c2nc(-c3ccccc3)nc(-c3ccc(-c4ccc(-n5c6ccccc6c6ccc7c8ccccc8oc7c65)cc4)cc3)n2)cc1]

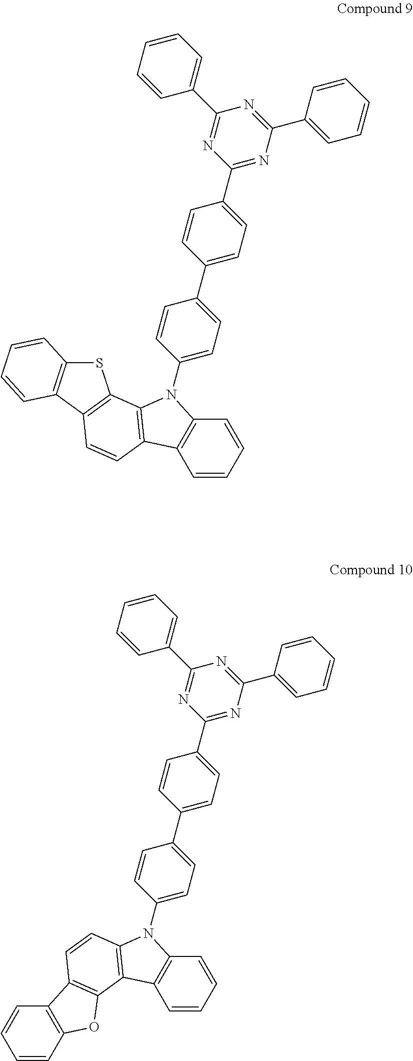 [SMILES: c1ccc(-c2nc(-c3ccccc3)nc(-c3ccc(-c4ccc(-n5c6ccccc6c6ccc7c8ccccc8sc7c65)cc4)cc3)n2)cc1 ; c1ccc(-c2nc(-c3ccccc3)nc(-c3ccc(-c4ccc(-n5c6ccccc6c6c7oc8ccccc8c7ccc65)cc4)cc3)n2)cc1]

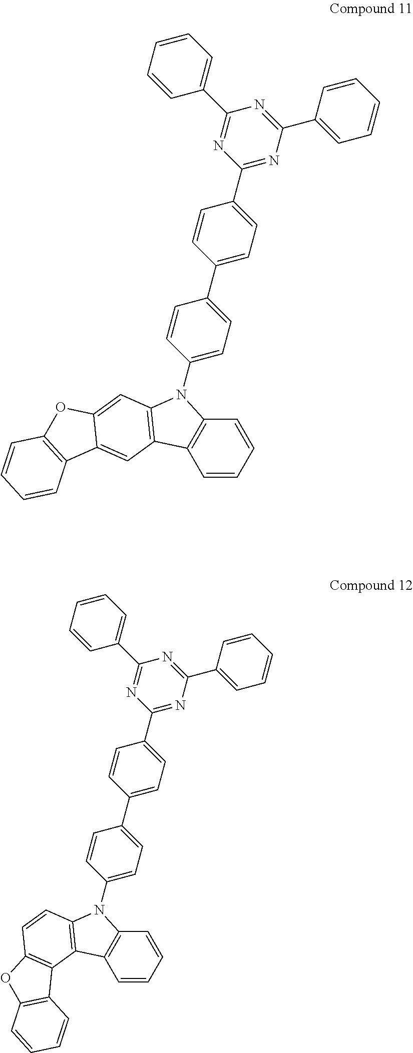 [SMILES: c1ccc(-c2nc(-c3ccccc3)nc(-c3ccc(-c4ccc(-n5c6ccccc6c6cc7c(cc65)oc5ccccc57)cc4)cc3)n2)cc1 ; c1ccc(-c2nc(-c3ccccc3)nc(-c3ccc(-c4ccc(-n5c6ccccc6c6c7c(ccc65)oc5ccccc57)cc4)cc3)n2)cc1]

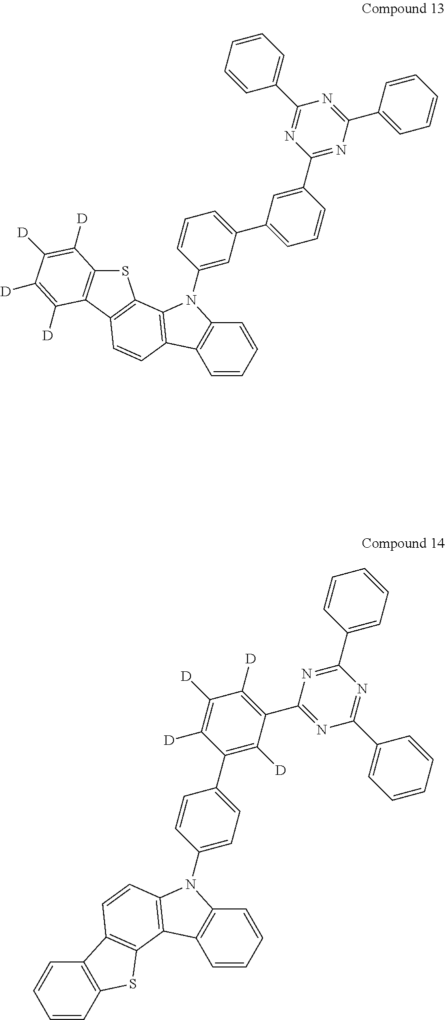 [SMILES: [2H]c1c([2H])c([2H])c2c(sc3c2ccc2c4ccccc4n(-c4cccc(-c5cccc(-c6nc(-c7ccccc7)nc(-c7ccccc7)n6)c5)c4)c23)c1[2H] ; [2H]c1c([2H])c(-c2ccc(-n3c4ccccc4c4c5sc6ccccc6c5ccc43)cc2)c([2H])c(-c2nc(-c3ccccc3)nc(-c3ccccc3)n2)c1[2H]]

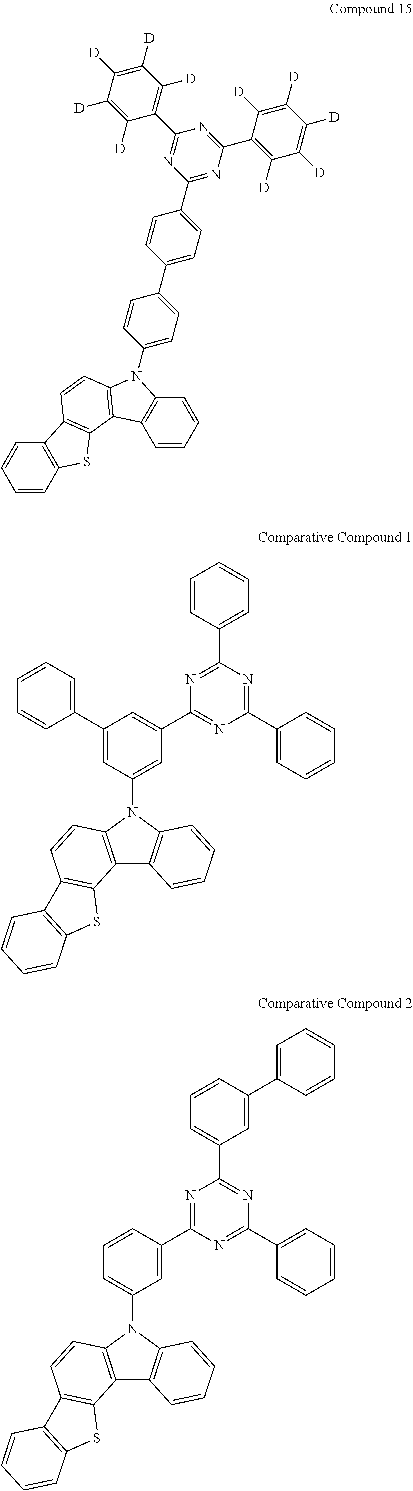 [SMILES: [2H]c1c([2H])c([2H])c(-c2nc(-c3ccc(-c4ccc(-n5c6ccccc6c6c7sc8ccccc8c7ccc65)cc4)cc3)nc(-c3c([2H])c([2H])c([2H])c([2H])c3[2H])n2)c([2H])c1[2H] ; c1ccc(-c2cc(-c3nc(-c4ccccc4)nc(-c4ccccc4)n3)cc(-n3c4ccccc4c4c5sc6ccccc6c5ccc43)c2)cc1 ; c1ccc(-c2cccc(-c3nc(-c4ccccc4)nc(-c4cccc(-n5c6ccccc6c6c7sc8ccccc8c7ccc65)c4)n3)c2)cc1]

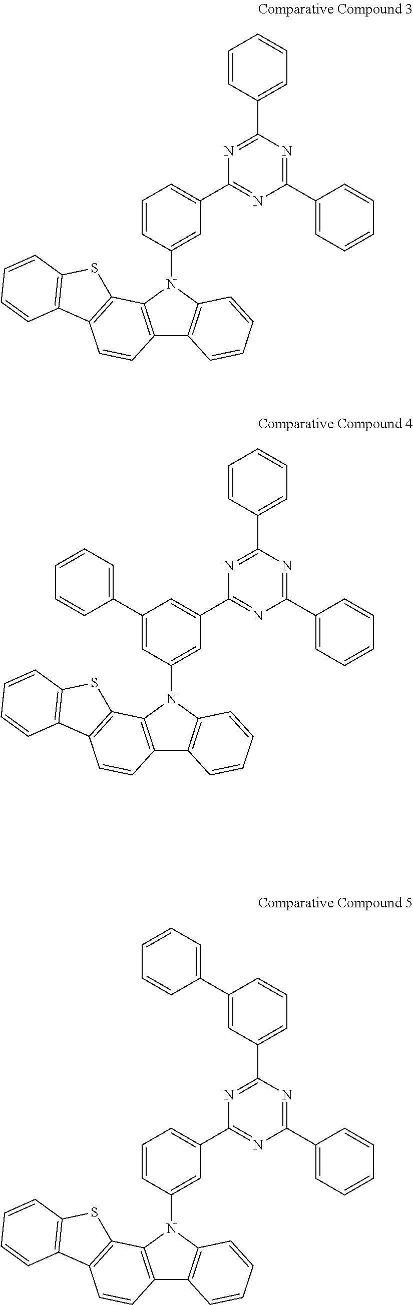 [SMILES: c1ccc(-c2nc(-c3ccccc3)nc(-c3cccc(-n4c5ccccc5c5ccc6c7ccccc7sc6c54)c3)n2)cc1 ; c1ccc(-c2cc(-c3nc(-c4ccccc4)nc(-c4ccccc4)n3)cc(-n3c4ccccc4c4ccc5c6ccccc6sc5c43)c2)cc1 ; c1ccc(-c2cccc(-c3nc(-c4ccccc4)nc(-c4cccc(-n5c6ccccc6c6ccc7c8ccccc8sc7c65)c4)n3)c2)cc1]

### Preparation Example 1: Compound 1

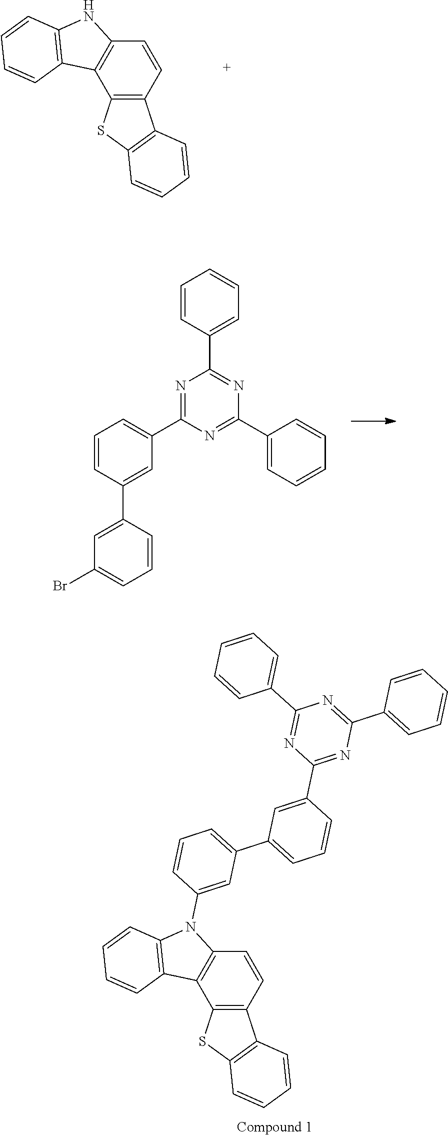 [SMILES: c1ccc2c(c1)[nH]c1ccc3c4ccccc4sc3c12 ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-n5c6ccccc6c6c7sc8ccccc8c7ccc65)c4)c3)n2)cc1]

In a nitrogen flushed 1000 ml three-necked round-bottomed flask 5H-[1]benzothieno[3,2-c]carbazole (9 g, 32.9 mmol), 2-(3′-bromo-[1,1′-biphenyl]-3-yl)-4,6-diphenyl-1,3,5-triazine (14.3 g, 32.9 mmol), tri-t-butylphosphonium tetrafluoroborate (0.38 g, 1.32 mmol), tris(dibenzylideneacetone)dipalladium(0) (0.6 g, 0.66 mmol) and sodium tert-butoxide (4.75 g, 50 mmol) were dissolved in 220 ml xylene under nitrogen. The reaction mixture was heated at 135° C. with an oil bath for 5 hours. After cooling down to room temperature, 220 ml methanol were added to the reaction mixture and stirred at room temperature for 1 hour. The product was recrystallised from xylene and filtered to give 18 g (83% yield) of Compound 1 as a white solid. The identification of Compound 1 was made by FD-MS (field desorption mass spectrometry), maximum ultraviolet absorption wavelength (UV(PhMe) Amax) in toluene, and maximum fluorescence wavelength (FL(PhMe, λex=315 nm) λmax) in toluene. The results are shown below.

FDMS: calcd. for C45H28N4S=656, found m/z=656 (M+)

### UV(PhMe) λmax: 357 nm

FL(PhMe, λex=315 nm) λmax: 361 nm

### Preparation Example: Compound 2

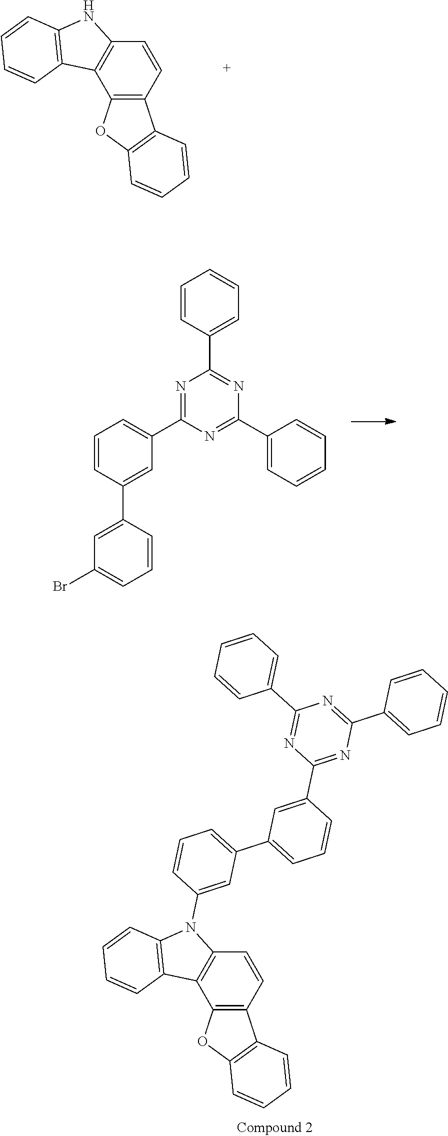 [SMILES: c1ccc2c(c1)[nH]c1ccc3c4ccccc4oc3c12 ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-n5c6ccccc6c6c7oc8ccccc8c7ccc65)c4)c3)n2)cc1]

The procedure of the synthesis of Compound 1 was repeated except for using 5H-benzofuro[3,2-c]carbazole in place of 5H-[1]benzothieno[3,2-c]carbazole. The obtained Compound 2 (81% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N₄O=640, found m/z=640 (M+)

### UV(PhMe) λmax: 352 nm

FL(PhMe, λex=330 nm) λmax: 420 nm

### Preparation Example 3: Compound 3

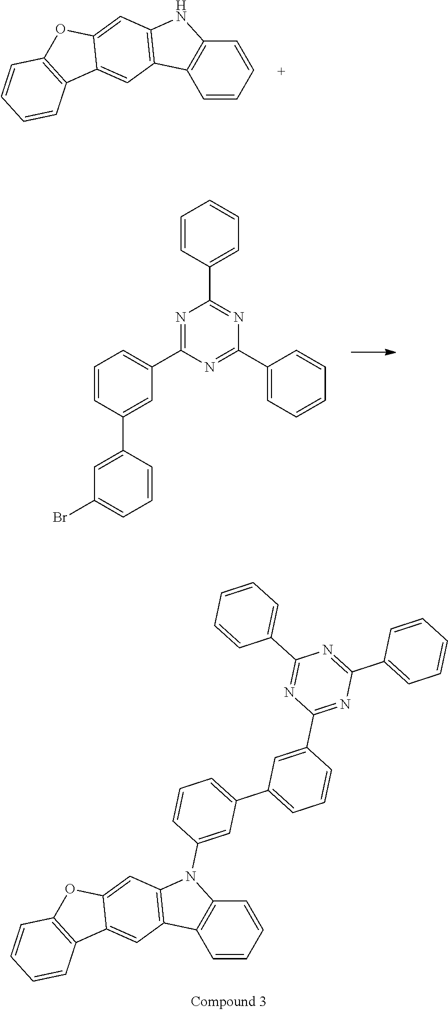 [SMILES: c1ccc2c(c1)[nH]c1cc3oc4ccccc4c3cc12 ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-n5c6ccccc6c6cc7c(cc65)oc5ccccc57)c4)c3)n2)cc1]

The procedure of the synthesis of Compound 1 was repeated except for using 7H-benzofuro[2,3-b]carbazole in place of 5H-[1]benzothieno[3,2-c]carbazole. The obtained Compound 3 (86% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N₄O=640, found m/z=640 (M+)

### UV(PhMe) λmax: 358 nm

FL(PhMe, λex=330 nm) λmax: 364 nm

### Preparation Example 4: Compound 4

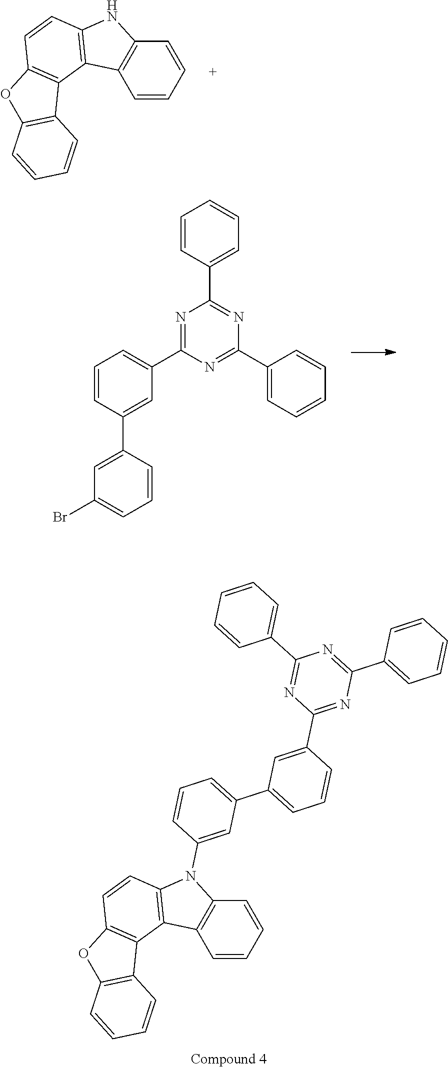 [SMILES: c1ccc2c(c1)[nH]c1ccc3oc4ccccc4c3c12 ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-n5c6ccccc6c6c7c(ccc65)oc5ccccc57)c4)c3)n2)cc1]

The procedure of the synthesis of Compound 1 was repeated except for using 8H-benzofuro[2,3-c]carbazole in place of 5H-[1]benzothieno[3,2-c]carbazole. The obtained Compound 4 (47% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4O=640, found m/z=640 (M+)

### UV(PhMe) λmax: 372 nm

FL(PhMe, λex=330 nm) λmax: 379 nm

### Preparation Example 5: Compound 5

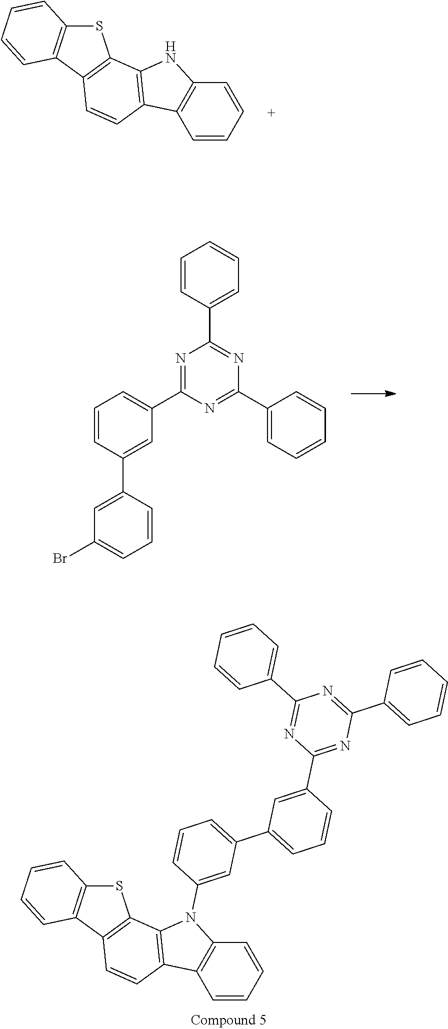 [SMILES: c1ccc2c(c1)[nH]c1c2ccc2c3ccccc3sc21 ; Brc1cccc(-c2cccc(-c3nc(-c4ccccc4)nc(-c4ccccc4)n3)c2)c1 ; c1ccc(-c2nc(-c3ccccc3)nc(-c3cccc(-c4cccc(-n5c6ccccc6c6ccc7c8ccccc8sc7c65)c4)c3)n2)cc1]

The procedure of the synthesis of Compound 1 was repeated except for using 12H-benzo[4,5]thieno[2,3-a]carbazole in place of 5H-[1]benzothieno[3,2-c]carbazole. The obtained Compound 5 (83% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4S=656, found m/z=656 (M+)

### UV(PhMe) λmax: 362 nm

FL(PhMe, λex=330 nm) λmax: 368 nm

### Preparation Example 6: Compound 6

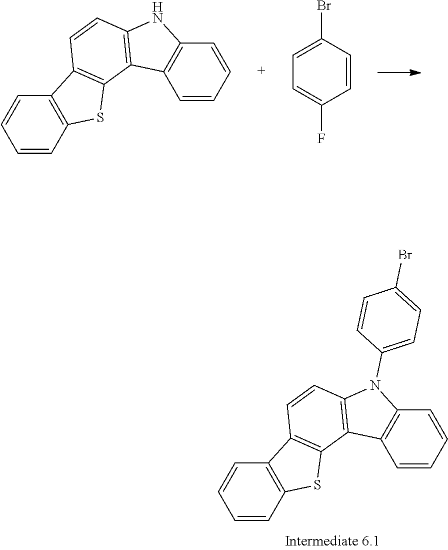 [SMILES: c1ccc2c(c1)[nH]c1ccc3c4ccccc4sc3c12 ; Fc1ccc(Br)cc1 ; Brc1ccc(-n2c3ccccc3c3c4sc5ccccc5c4ccc32)cc1]

5H-[1]benzo[4,5]thieno[3,2-c]carbazole (10 g, 36.6 mmol) was combined with 1-bromo-4-fluorobenzene (24.1 ml, 220 mmol) and K₃PO₄ (23.3 g, 110 mmol) in a 200 ml flask. N-methyl pyrrolidine (85 ml) was then added and the reaction mixture allowed to stir at an oil bath temperature of 170° C. for 20 hours. The reaction mixture was then allowed to cool to 90° C. and the insoluble material was filtered off. The filtrate thus obtained was concentrated under reduced pressure. This residue was then taken up in dichloromethane and washed with water, dried over MgSO₄ and the solvent was evaporated. The crude residue was then suspended in methanol and stirred at room temperature. The product Intermediate 6.1 (13.5 g, 86% yield, off-white solid) was then filtered and dried, characterized by FD-MS (field desorption mass spectrometry) and used without further purification.

FD-MS: calcd. for C24H14BrNS=428, found m/z=428 (M+)

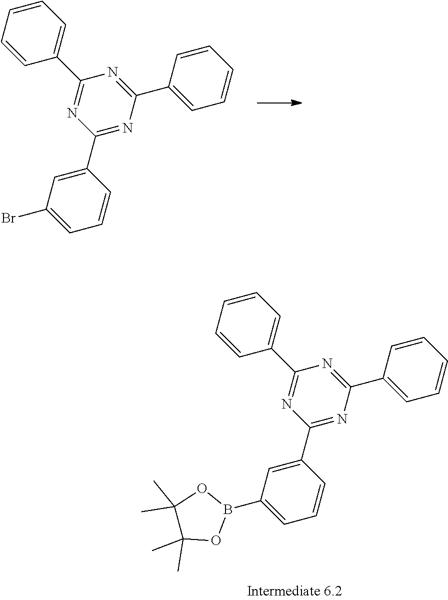 [SMILES: Brc1cccc(-c2nc(-c3ccccc3)nc(-c3ccccc3)n2)c1 ; CC1(C)OB(c2cccc(-c3nc(-c4ccccc4)nc(-c4ccccc4)n3)c2)OC1(C)C]

2-(3-bromophenyl)-4,6-diphenyl-1,3,5-triazine (10 g, 25.8 mmol) was combined with bis(pinacolato)diboron (9.8 g, 38.6 mmol) and sodium acetate (4.2 g, 51.5 mmol) in anhydrous dioxane (150 ml) in a degassed 3 neck round bottom flask. PdC₁₂(dppf)-CH₂Cl₂adduct (1 g, 1.3 mmol) was then added and the reaction mixture was heated at an oil bath temperature of 100° C. for 20 hours. The reaction mixture was then allowed to cool to room temperature and the solvent was evaporated under reduced pressure. The crude residue was then dissolved in CH₂Cl₂ and washed with water, dried over anhydrous MgSO₄ and solvent was evaporated. The crude product was then suspended in methanol and stirred at room temperature for 1 hour. The product Intermediate 6.2 (9.8 g, 82% yield, brown solid) was collected by filtration, characterized by FD-MS (field desorption mass spectrometry) and used without further purification.

FD-MS: calcd. for C27H26BN302=435, found m/z=435 (M+)

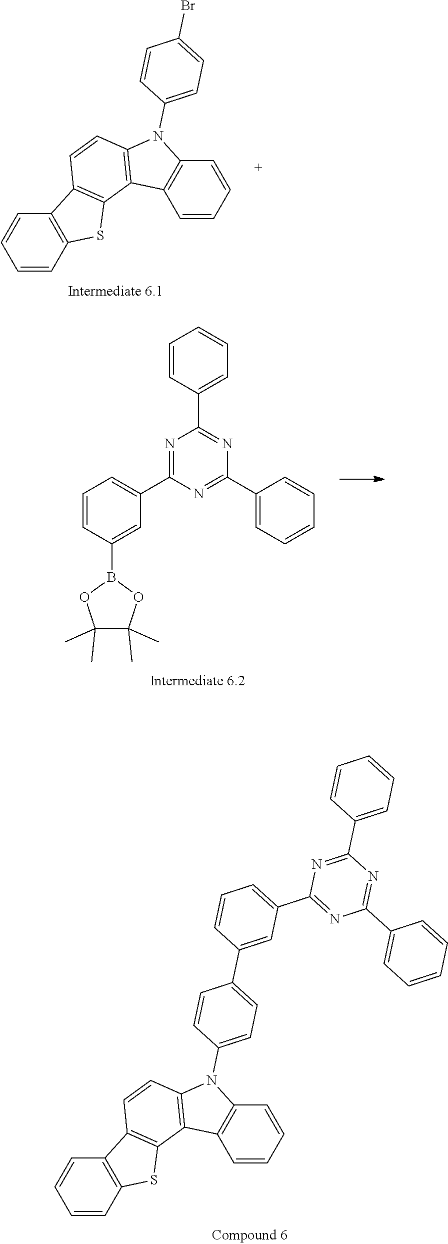 [SMILES: Brc1ccc(-n2c3ccccc3c3c4sc5ccccc5c4ccc32)cc1 ; CC1(C)OB(c2cccc(-c3nc(-c4ccccc4)nc(-c4ccccc4)n3)c2)OC1(C)C ; c1ccc(-c2nc(-c3ccccc3)nc(-c3cccc(-c4ccc(-n5c6ccccc6c6c7sc8ccccc8c7ccc65)cc4)c3)n2)cc1]

Intermediate 6.1 (6.5 g, 15.2 mmol) was combined with Intermediate 6.2 (7 g, 16.1 mmol) and K₂CO₃ (5.2 g, 37.9 mmol) in solution of toluene/water/ethanol (200 ml, 5:1:1.7) in a pre-dried 3 neck round bottom flask under N₂. Pd(OAc)₂ (68 mg, 0.68 mmol) and dicyclohexyl(2′,4′,6′-triisopropyl[1,1-biphenyl]-2-yl)phosphane (0.3 g, 0.6 mmol) was then added. The resulting reaction mixture was heated at an oil bath temperature of 90° C. for 2 hours. The reaction mixture was allowed to cool and the crude product was collected by filtration. The crude product was then washed with toluene, water and finally methanol. After recrystallisation from chlorobenzene, the purified Compound 6 (4.9 g, 49% yield, white solid) was thus isolated. Compound 6 was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4S=656, found m/z=656 (M+)

### UV(PhMe) λmax: 358 nm

FL(PhMe, λex=330 nm) λmax: 406 nm

### Preparation Example 7: Compound 7

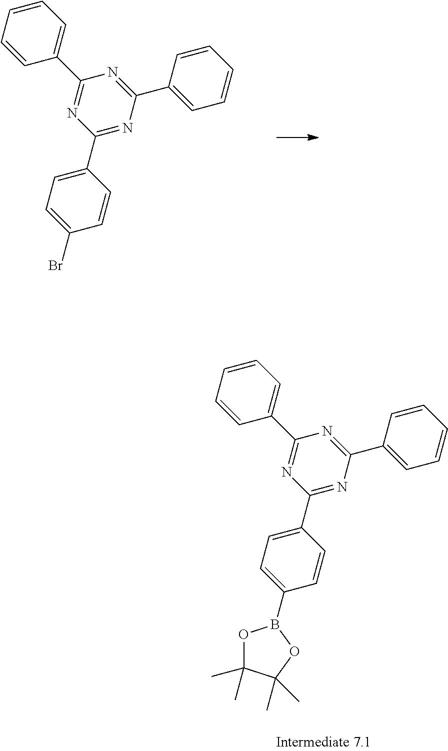 [SMILES: Brc1ccc(-c2nc(-c3ccccc3)nc(-c3ccccc3)n2)cc1 ; CC1(C)OB(c2ccc(-c3nc(-c4ccccc4)nc(-c4ccccc4)n3)cc2)OC1(C)C]

The procedure of the synthesis of Intermediate 6.2 was repeated except for using 2-(4-bromophenyl)-4,6-diphenyl-1,3,5-triazine in place of 2-(3-bromophenyl)-4,6-diphenyl-1,3,5-triazine. The obtained Intermediate 7.1 (78% yield, brown solid) was characterized by FD-MS (field desorption mass spectrometry).

FD-MS: calcd. for C27H26BN302=435, found m/z=435 (M+)

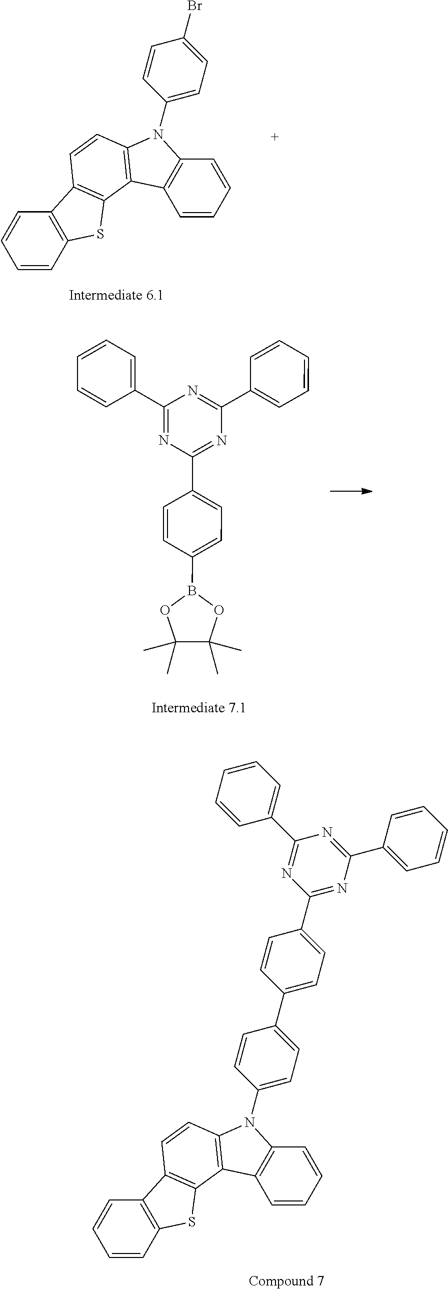 [SMILES: Brc1ccc(-n2c3ccccc3c3c4sc5ccccc5c4ccc32)cc1 ; CC1(C)OB(c2ccc(-c3nc(-c4ccccc4)nc(-c4ccccc4)n3)cc2)OC1(C)C ; c1ccc(-c2nc(-c3ccccc3)nc(-c3ccc(-c4ccc(-n5c6ccccc6c6c7sc8ccccc8c7ccc65)cc4)cc3)n2)cc1]

The procedure of the synthesis of Compound 6 was repeated except for using Intermediate 7.1 in place of Intermediate 6.2. The obtained Compound 7 (53% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.
FD-MS: calcd. for C45H28N4S=656, found m/z=656 (M+)

### UV(PhMe) λmax: 359 nm

FL(PhMe, λex=330 nm) λmax: 425 nm

### Preparation Example 8: Compound 8

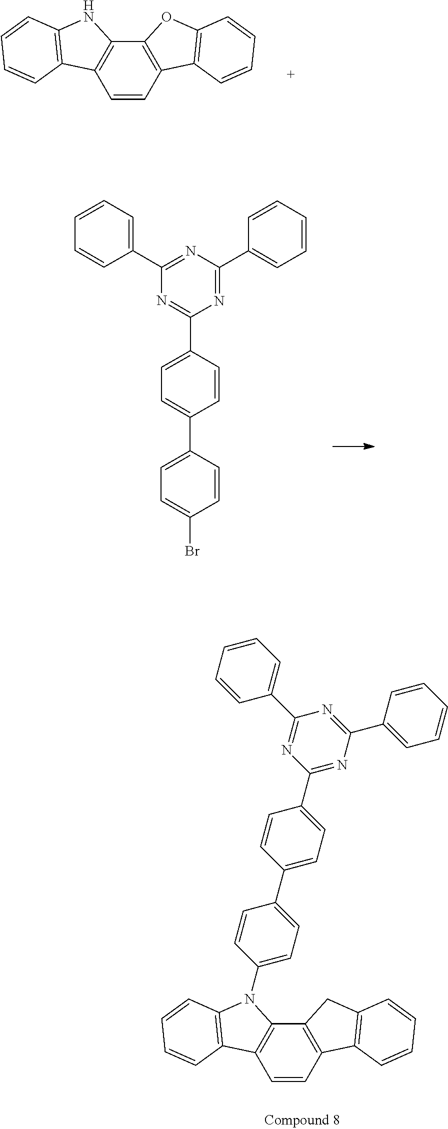 [SMILES: c1ccc2c(c1)[nH]c1c2ccc2c3ccccc3oc21 ; Brc1ccc(-c2ccc(-c3nc(-c4ccccc4)nc(-c4ccccc4)n3)cc2)cc1 ; c1ccc(-c2nc(-c3ccccc3)nc(-c3ccc(-c4ccc(-n5c6ccccc6c6ccc7c(c65)Cc5ccccc5-7)cc4)cc3)n2)cc1]

The procedure of the synthesis of Compound 1 was repeated except for using 2-(4′-bromo-[1,1-biphenyl]-4-yl)-4,6-diphenyl-1,3,5-triazine in place of 2-(3′-bromo-[1,1′-biphenyl]-3-yl)-4,6-diphenyl-1,3,5-triazine and 12H-benzofuro[2,3-a]carbazole in place of 5H-[1]benzothieno[3,2-c]carbazole. The obtained Compound 8 (80% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4O=640, found m/z=640 (M+)

### UV(PhMe) λmax: 354 nm

FL(PhMe, λex=330 nm) λmax: 421 nm

### Preparation Example 9: Compound 9

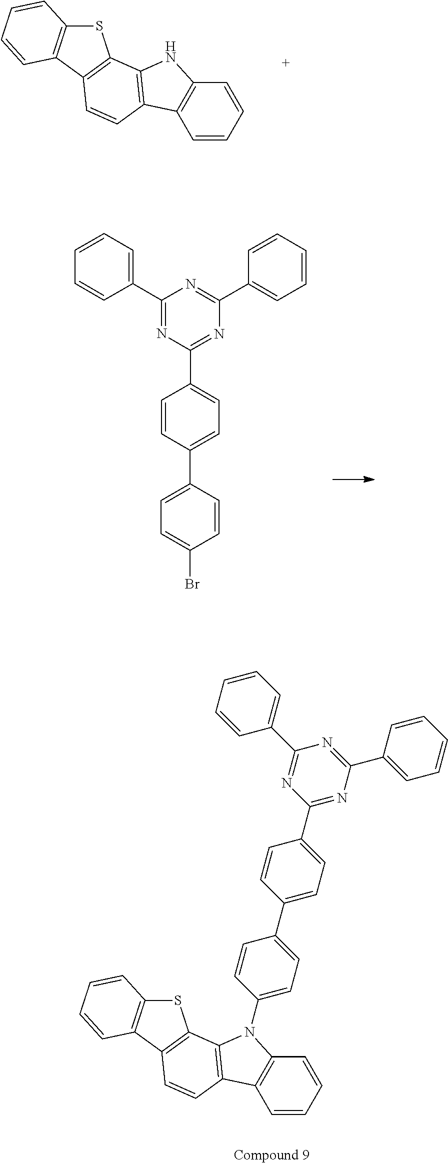 [SMILES: c1ccc2c(c1)[nH]c1c2ccc2c3ccccc3sc21 ; Brc1ccc(-c2ccc(-c3nc(-c4ccccc4)nc(-c4ccccc4)n3)cc2)cc1 ; c1ccc(-c2nc(-c3ccccc3)nc(-c3ccc(-c4ccc(-n5c6ccccc6c6ccc7c8ccccc8sc7c65)cc4)cc3)n2)cc1]

The procedure of the synthesis of Compound 5 was repeated except for using 2-(4′-bromo-[1,1′-biphenyl]-4-yl)-4,6-diphenyl-1,3,5-triazine in place of 2-(3′-bromo-[1,1′-biphenyl]-3-yl)-4,6-diphenyl-1,3,5-triazine. The obtained Compound 9 (56% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4S=656, found m/z=656 (M+)

### UV(PhMe) λmax: 364 nm

FL(PhMe, λex=330 nm) λmax: 427 nm

### Preparation Example 10: Compound 10

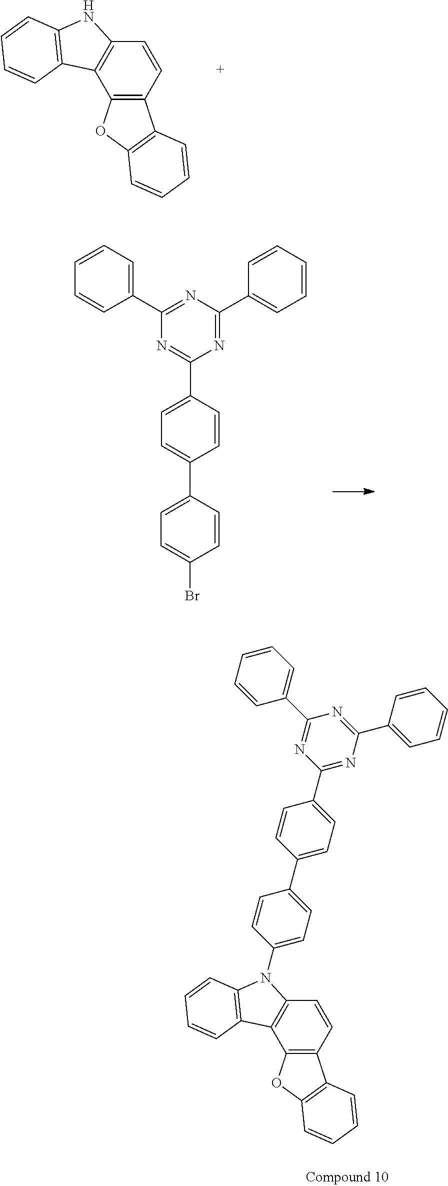 [SMILES: c1ccc2c(c1)[nH]c1ccc3c4ccccc4oc3c12 ; Brc1ccc(-c2ccc(-c3nc(-c4ccccc4)nc(-c4ccccc4)n3)cc2)cc1 ; c1ccc(-c2nc(-c3ccccc3)nc(-c3ccc(-c4ccc(-n5c6ccccc6c6c7oc8ccccc8c7ccc65)cc4)cc3)n2)cc1]

The procedure of the synthesis of Compound 2 was repeated except for using 2-(4′-bromo-[1,1′-biphenyl]-4-yl)-4,6-diphenyl-1,3,5-triazine in place of 2-(3′-bromo-[1,1′-biphenyl]-3-yl)-4,6-diphenyl-1,3,5-triazine. The obtained Compound 10 (71% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4O=640, found m/z=640 (M+)

### UV(PhMe) λmax: 353 nm

FL(PhMe, λex=330 nm) λmax: 427 nm

### Preparation Example 11: Compound 11

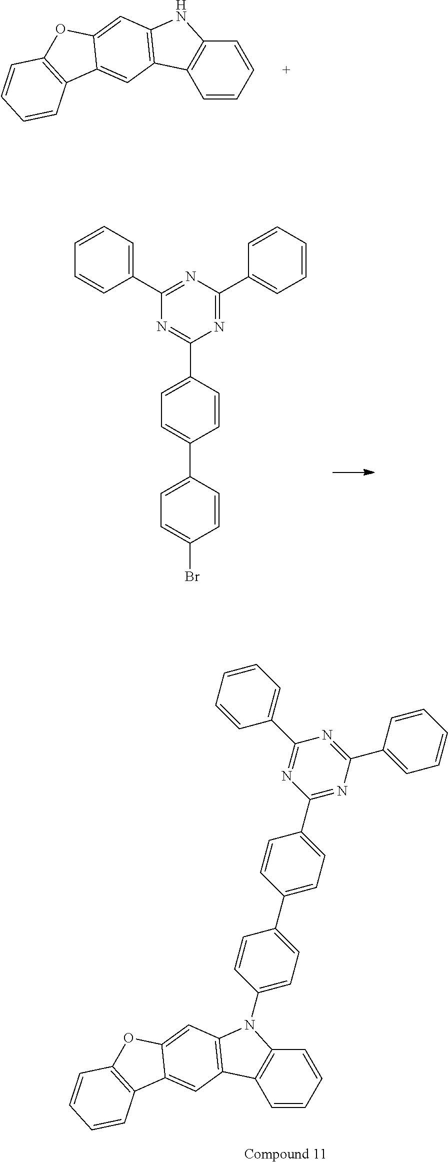 [SMILES: c1ccc2c(c1)[nH]c1cc3oc4ccccc4c3cc12 ; Brc1ccc(-c2ccc(-c3nc(-c4ccccc4)nc(-c4ccccc4)n3)cc2)cc1 ; c1ccc(-c2nc(-c3ccccc3)nc(-c3ccc(-c4ccc(-n5c6ccccc6c6cc7c(cc65)oc5ccccc57)cc4)cc3)n2)cc1]

The procedure of the synthesis of Compound 3 was repeated except for using 2-(4′-bromo-[1,1′-biphenyl]-4-yl)-4,6-diphenyl-1,3,5-triazine in place of 2-(3′-bromo-[1,1′-biphenyl]-3-yl)-4,6-diphenyl-1,3,5-triazine. The obtained Compound 11 (69% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4O=640, found m/z=640 (M+)

### UV(PhMe) λmax: 358 nm

FL(PhMe, λex=330 nm) λmax: 425 nm

### Preparation Example 12: Compound 12

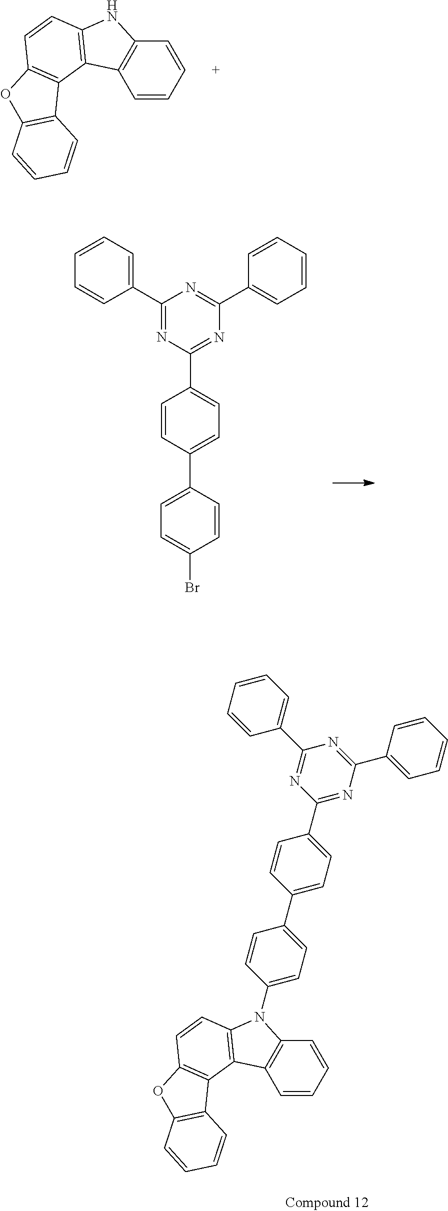 [SMILES: c1ccc2c(c1)[nH]c1ccc3oc4ccccc4c3c12 ; Brc1ccc(-c2ccc(-c3nc(-c4ccccc4)nc(-c4ccccc4)n3)cc2)cc1 ; c1ccc(-c2nc(-c3ccccc3)nc(-c3ccc(-c4ccc(-n5c6ccccc6c6c7c(ccc65)oc5ccccc57)cc4)cc3)n2)cc1]

The procedure of the synthesis of Compound 4 was repeated except for using 2-(4′-bromo-[1,1′-biphenyl]-4-yl)-4,6-diphenyl-1,3,5-triazine in place of 2-(3′-bromo-[1,1′-biphenyl]-3-yl)-4,6-diphenyl-1,3,5-triazine. The obtained Compound 12 (84% yield, white solid) was characterized by FD-MS (field desorption mass spectrometry), maximum ultraviolet absorption wavelength (UV(PhMe) λmax) in toluene, and maximum fluorescence wavelength (FL(PhMe, λex=330 nm) λmax) in toluene. The results are shown below.

FD-MS: calcd. for C45H28N4O=640, found m/z=640 (M+)

### UV(PhMe) λmax: 374 nm

FL(PhMe, λex=330 nm) λmax: 428 nm

### Preparation Example 13: Compound 13

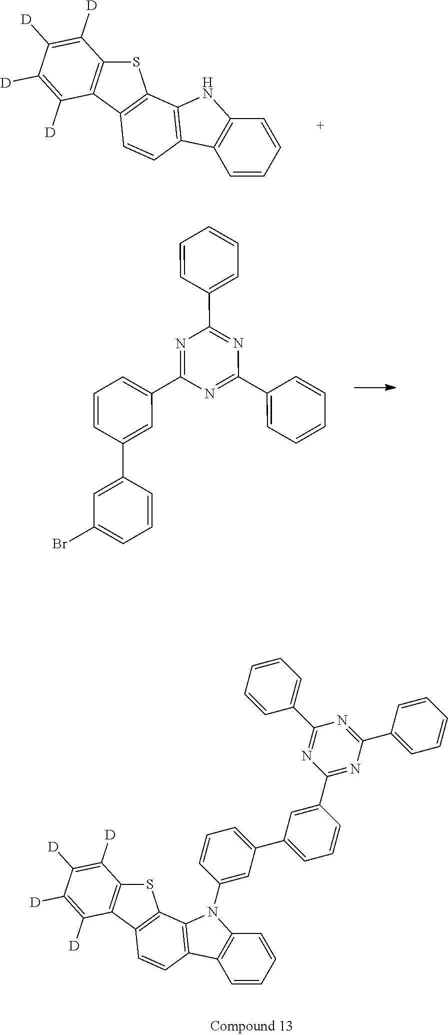 [SMILES: [2H]c1c([2H])c([2H])c2c(sc3c2ccc2c4ccccc4[nH]c23)c1[2H] ; Brc1cccc(-c2cccc(-c3nc(-c4ccccc4)nc(-c4ccccc4)n3)c2)c1 ; [2H]c1c([2H])c([2H])c2c(sc3c2ccc2c4ccccc4n(-c4cccc(-c5cccc(-c6nc(-c7ccccc7)nc(-c7ccccc7)n6)c5)c4)c23)c1[2H]]

The procedure of the synthesis of Compound 5 was repeated except for using 12H-benzo[4,5]thieno[2,3-a]carbazole-7, 8, 9,10-d4 in place of 12H-benzo[4,5]thieno[2,3-a]carbazole. The obtained Compound 13 (79% yield, white solid) was characterized by FD-MS (field desorption mass spectrometry). The results are shown below.

FD-MS: calcd. for C45H24D4N4S=660, found m/z=660 (M+)

### Preparation Example 14: Compound 14

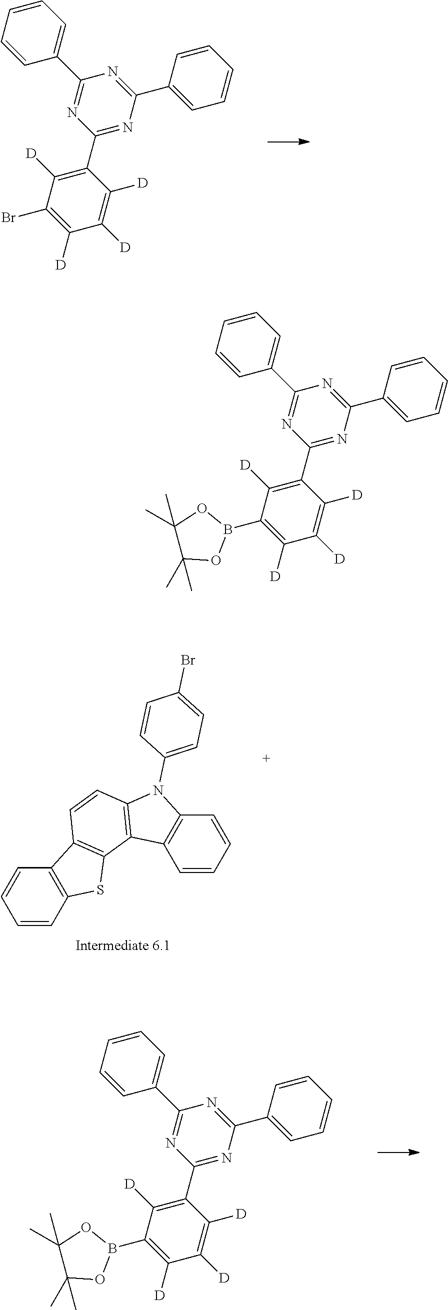 [SMILES: [2H]c1c([2H])c(Br)c([2H])c(-c2nc(-c3ccccc3)nc(-c3ccccc3)n2)c1[2H] ; [2H]c1c([2H])c(B2OC(C)(C)C(C)(C)O2)c([2H])c(-c2nc(-c3ccccc3)nc(-c3ccccc3)n2)c1[2H] ; Brc1ccc(-n2c3ccccc3c3c4sc5ccccc5c4ccc32)cc1 ; [2H]c1c([2H])c(B2OC(C)(C)C(C)(C)O2)c([2H])c(-c2nc(-c3ccccc3)nc(-c3ccccc3)n2)c1[2H]]

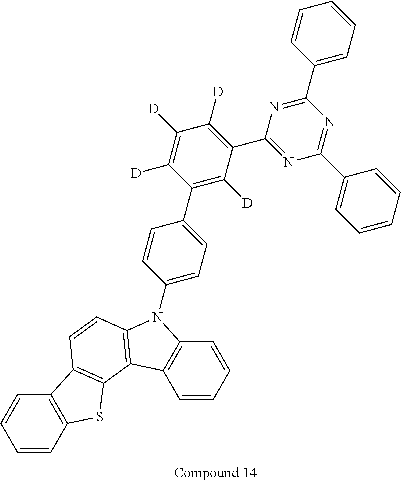 [SMILES: [2H]c1c([2H])c(-c2ccc(-n3c4ccccc4c4c5sc6ccccc6c5ccc43)cc2)c([2H])c(-c2nc(-c3ccccc3)nc(-c3ccccc3)n2)c1[2H]]

The procedure of the synthesis of Compound 6 was repeated except for using 2-(5-bromophenyl-2,3,4,6-d4)-4,6-diphenyl-1,3,5-triazine in place of 2-(3-bromophenyl)-4,6-diphenyl-1,3,5-triazine. The obtained Compound 14 (white solid) was characterized by FD-MS (field desorption mass spectrometry). The results are shown below.

FD-MS: calcd. for C45H24D4N4S=660, found m/z=660 (M+

### Preparation Example 15: Compound 15

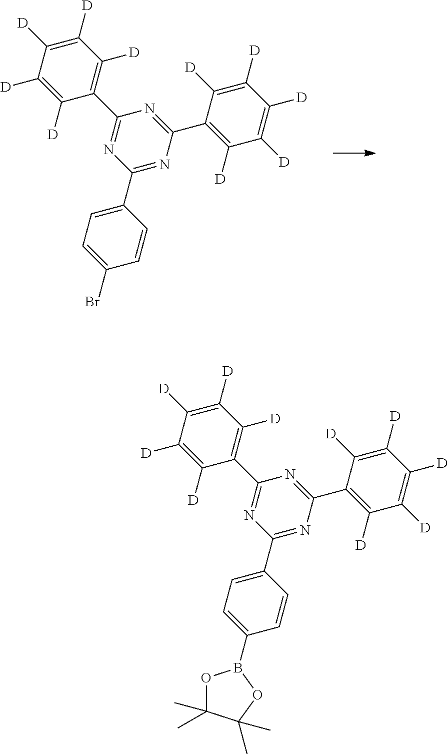 [SMILES: [2H]c1c([2H])c([2H])c(-c2nc(-c3ccc(Br)cc3)nc(-c3c([2H])c([2H])c([2H])c([2H])c3[2H])n2)c([2H])c1[2H] ; [2H]c1c([2H])c([2H])c(-c2nc(-c3ccc(B4OC(C)(C)C(C)(C)O4)cc3)nc(-c3c([2H])c([2H])c([2H])c([2H])c3[2H])n2)c([2H])c1[2H]]

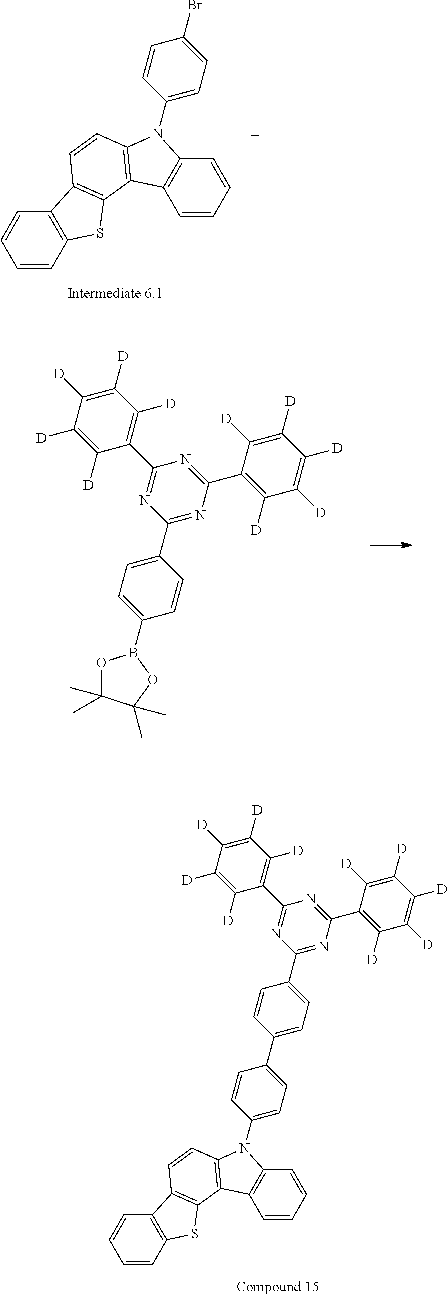 [SMILES: Brc1ccc(-n2c3ccccc3c3c4sc5ccccc5c4ccc32)cc1 ; [2H]c1c([2H])c([2H])c(-c2nc(-c3ccc(B4OC(C)(C)C(C)(C)O4)cc3)nc(-c3c([2H])c([2H])c([2H])c([2H])c3[2H])n2)c([2H])c1[2H] ; [2H]c1c([2H])c([2H])c(-c2nc(-c3ccc(-c4ccc(-n5c6ccccc6c6c7sc8ccccc8c7ccc65)cc4)cc3)nc(-c3c([2H])c([2H])c([2H])c([2H])c3[2H])n2)c([2H])c1[2H]]

The procedure of the synthesis of Compound 7 was repeated except for using 2-(4-bromophenyl)-4,6-di(phenyl-2,3,4,5,6-d5)-1,3,5-triazine in place of 2-(4-bromophenyl)-4,6-diphenyl-1,3,5-triazine. The obtained Compound 15 (white solid) was characterized by FD-MS (field desorption mass spectrometry). The results are shown below.

FD-MS: calcd. for C45H18D10N4S=666, found m/z=666 (M+)

### Preparation Example 16: Comparative Compound 1

### Step 1

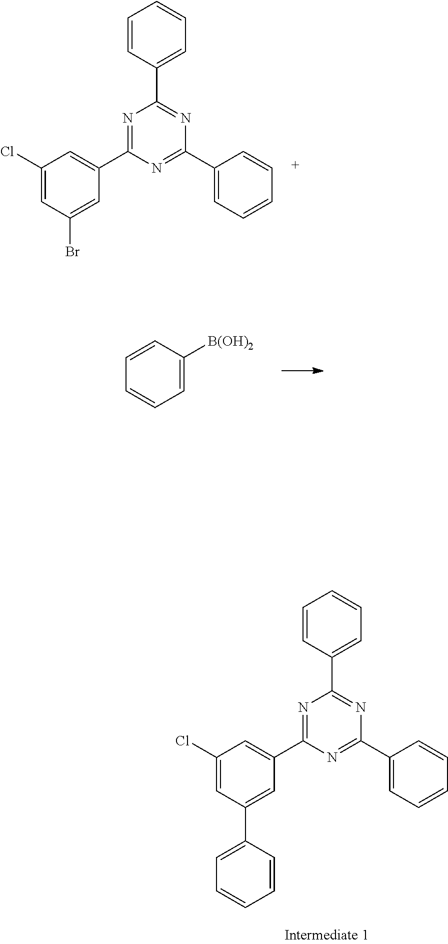 [SMILES: Clc1cc(Br)cc(-c2nc(-c3ccccc3)nc(-c3ccccc3)n2)c1 ; OB(O)c1ccccc1 ; Clc1cc(-c2ccccc2)cc(-c2nc(-c3ccccc3)nc(-c3ccccc3)n2)c1]

In a nitrogen flushed 2000 ml three-necked round-bottomed flask, 2-(3-bromo-5-chlorophenyl)-4,6-diphenyl-1,3,5-triazine (84.5 g, 0.20 mol) and phenylboronic acid (26.8 g, 0.22 mol) were dissolved in toluene (600 ml) under nitrogen. 2M sodium carbonate solution (300 ml, 0.60 mol) and tetrakis(triphenylphosphine)palladium(0) (6.93 g, 0.005 mol) were added to the reaction mixture. The reaction mixture was heated in an oil bath at 100° C. for 24 hours. After cooling down to room temperature, water was added to the reaction mixture followed by extraction with toluene. The combined organic layers were concentrated. The crude product was added to a silica gel column and was eluted with toluene and hexane to give 55 g of the Intermediate 1 as white solid (65% yield). The identification of the Intermediate 1 was made by FD-MS (field desorption mass spectrometry) analysis.

FDMS: calcd. for C27H18CIN3=420, found m/z=420 (M+)

### Step 2

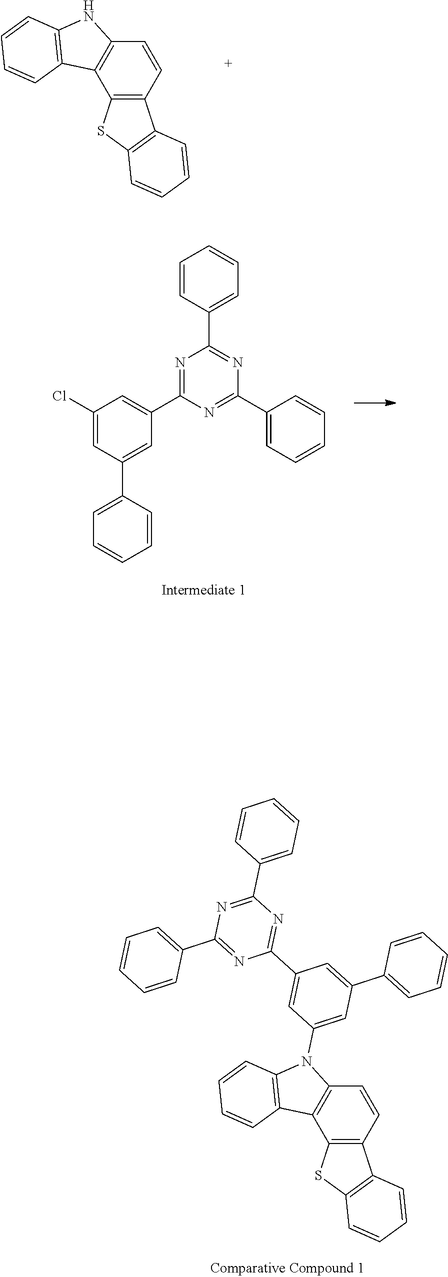 [SMILES: c1ccc2c(c1)[nH]c1ccc3c4ccccc4sc3c12 ; Clc1cc(-c2ccccc2)cc(-c2nc(-c3ccccc3)nc(-c3ccccc3)n2)c1 ; c1ccc(-c2cc(-c3nc(-c4ccccc4)nc(-c4ccccc4)n3)cc(-n3c4ccccc4c4c5sc6ccccc6c5ccc43)c2)cc1]

In a nitrogen flushed 1000 ml three-necked round-bottomed flask 5H-[1]benzothieno[3,2-c]carbazole (9.4 g, 34 mmol), Intermediate 1 (14.4 g, 34 mmol), 2-dicyclohexylphosphino-2′,4′, 6′-triisopropylbiphenyl (0.65 g, 1.36 mmol), tris(dibenzylideneacetone)dipalladium(0) (0.63 g, 0.688 mmol) and sodium tert-butoxide (4.9 g, 50 mmol) were dissolved in 240 ml xylene under nitrogen. The reaction mixture was heated at 135° C. with an oil bath for 3 hours. After cooling down to room temperature, 150 ml methanol were added to the reaction mixture. The reaction mixture was filtered and the precipitate was washed with methanol. The precipitate was added to a silica gel column and was eluted with hot toluene, and then washed with hot xylene twice to give 20 g of Comparative Compound 1 as yellow solid (90% yield). The identification of Comparative Compound 1 was made by FD-MS (field desorption mass spectrometry), maximum ultraviolet absorption wavelength (UV(PhMe) λmax) in toluene, and maximum fluorescence wavelength (FL(PhMe, λex=315 nm) λmax) in toluene. The results are shown below.

FDMS: calcd. for C45H28N4S=656, found m/z=656 (M+)

### UV(PhMe) λmax: 356 nm

FL(PhMe, λex=315 nm) λmax: 446 nm

### Preparation Example 17: Comparative Compound 2

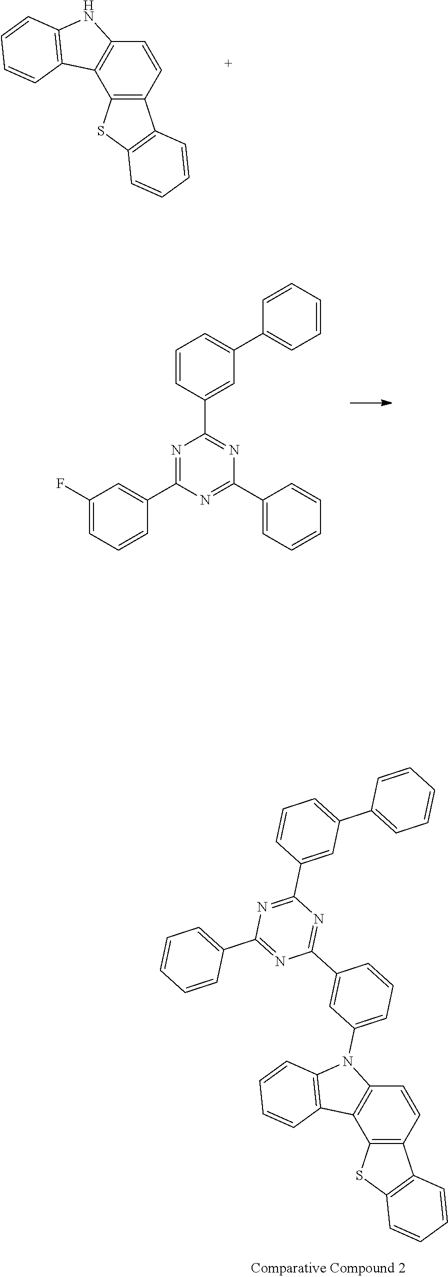 [SMILES: c1ccc2c(c1)[nH]c1ccc3c4ccccc4sc3c12 ; Fc1cccc(-c2nc(-c3ccccc3)nc(-c3cccc(-c4ccccc4)c3)n2)c1 ; c1ccc(-c2cccc(-c3nc(-c4ccccc4)nc(-c4cccc(-n5c6ccccc6c6c7sc8ccccc8c7ccc65)c4)n3)c2)cc1]

5H-[1]benzothieno[3,2-c]carbazole (2 g, 7.4 mmol) was combined with 2-([1,1′-biphenyl]-3-yl)-4-(3-fluorophenyl)-6-phenyl-1,3,5-triazine (2 g, 4.96 mmol), Cs₂CO₃ (6.5 g, 19.8 mmol) in N-methyl-pyrrolidinone (100 ml) and the resulting mixture was heated at an oil bath temperature of 190° C. overnight. The reaction was cooled to room temperature and diluted with ethyl acetate and washed with water. The solution was concentrated and the desired product crystallised out of solution. The product was finally washed with toluene and Comparative Compound 2 isolated as a pale yellow solid (2.9 g, 91% yield). The identification of Comparative Compound 2 was made by FD-MS (field desorption mass spectrometry), maximum ultraviolet absorption wavelength (UV(PhMe) λmax) in toluene, and maximum fluorescence wavelength (FL(PhMe, λex=322 nm) λmax) in toluene. The results are shown below.

FDMS: calcd. for C45H28N4S=656, found m/z=656 (M+)

### UV(PhMe) λmax: 356 nm

FL(PhMe, λex=322 nm) λmax: 459 nm

### Preparation Example 18: Comparative Compound 3

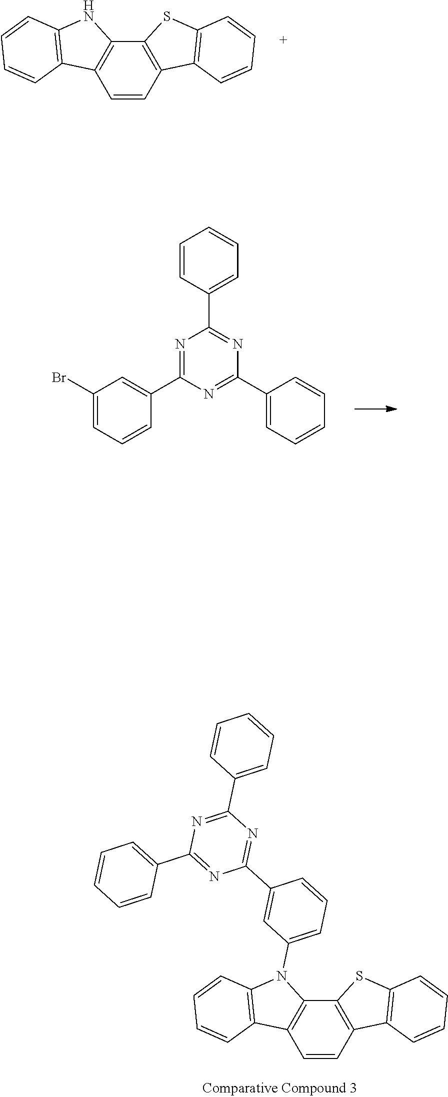 [SMILES: c1ccc2c(c1)[nH]c1c2ccc2c3ccccc3sc21 ; Brc1cccc(-c2nc(-c3ccccc3)nc(-c3ccccc3)n2)c1 ; c1ccc(-c2nc(-c3ccccc3)nc(-c3cccc(-n4c5ccccc5c5ccc6c7ccccc7sc6c54)c3)n2)cc1]

The procedure of the synthesis of Comparative Compound 1 was repeated except for using 12H-benzo[4,5]thieno[2,3-a]carbazole in place of 5H-[1]benzothieno[3,2-c]carbazole, for using 2-(3-bromophenyl)-4,6-diphenyl-1,3,5-triazine in place of Intermediate 1 and 4,5-bis(diphenylphosphino)-9,9-dimethylxanthene in place of 2-dicyclohexylphosphino-2′,4′,6′-triisopropylbiphenyl. The obtained Comparative Compound 3 (57% yield, white solid) was identified by FD-MS (field desorption mass spectrometry), maximum ultraviolet absorption wavelength (UV(PhMe) λmax) in toluene, and maximum fluorescence wavelength (FL(PhMe, λex=322 nm) λmax) in toluene. The results are shown below.

FDMS: calcd. for C39H24N4S=581, found m/z=581 (M+)

### UV(PhMe) λmax: 362 nm

FL(PhMe, λex=322 nm) λmax: 463 nm

### Preparation Example 19: Comparative Compound 4

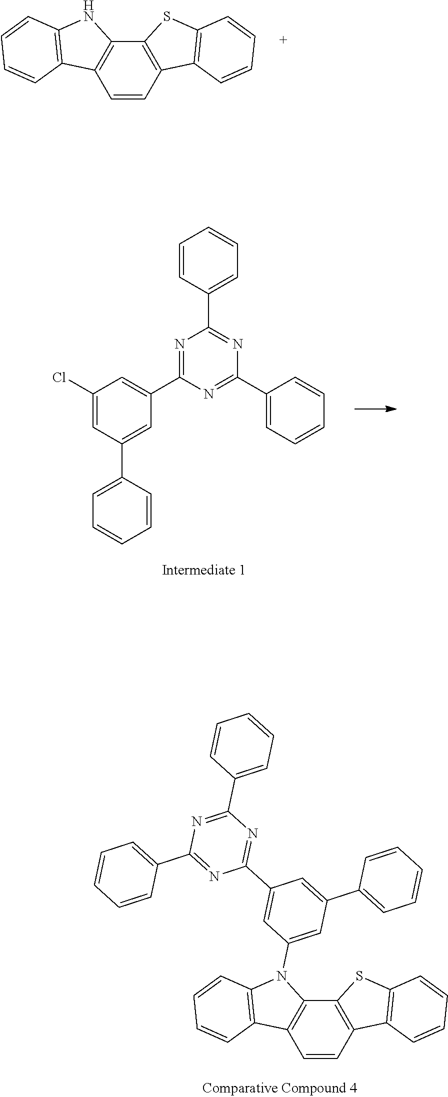 [SMILES: c1ccc2c(c1)[nH]c1c2ccc2c3ccccc3sc21 ; Clc1cc(-c2ccccc2)cc(-c2nc(-c3ccccc3)nc(-c3ccccc3)n2)c1 ; c1ccc(-c2cc(-c3nc(-c4ccccc4)nc(-c4ccccc4)n3)cc(-n3c4ccccc4c4ccc5c6ccccc6sc5c43)c2)cc1]

The procedure of the synthesis of Comparative Compound 1 was repeated except for using 12H-benzo[4,5]thieno[2,3-a]carbazole in place of 5H-k[1]benzothieno[3,2-c]carbazole. The obtained Comparative Compound 4 (69% yield, yellow solid) was identified by FD-MS (field desorption mass spectrometry), maximum ultraviolet absorption wavelength (UV(PhMe) λmax) in toluene, and maximum fluorescence wavelength (FL(PhMe, λex=322 nm) λmax) in toluene. The results are shown below.

FDMS: calcd. for C45H28N4S=656, found m/z=656 (M+)

### UV(PhMe) λmax: 365 nm

FL(PhMe, λex=322 nm) λmax: 450 nm

### Preparation Example 20: Comparative Compound 5

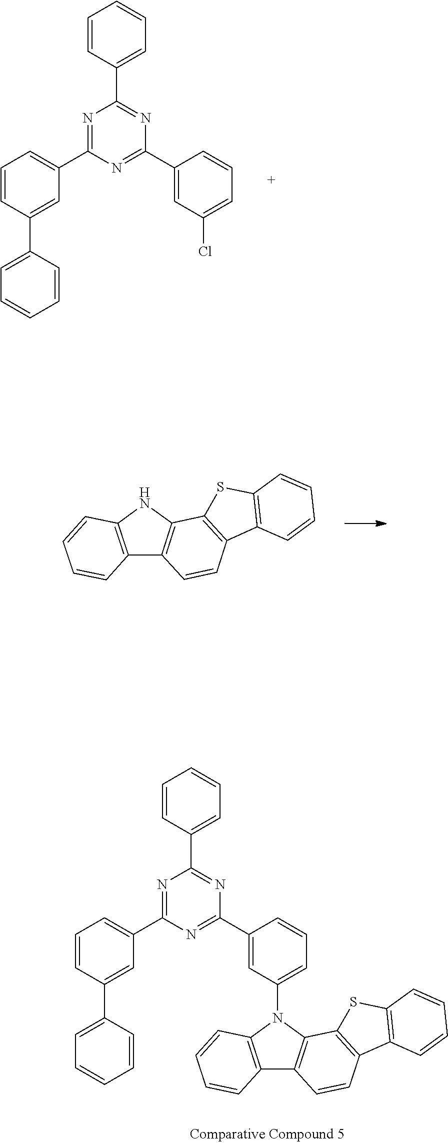 [SMILES: Clc1cccc(-c2nc(-c3ccccc3)nc(-c3cccc(-c4ccccc4)c3)n2)c1 ; c1ccc2c(c1)[nH]c1c2ccc2c3ccccc3sc21 ; c1ccc(-c2cccc(-c3nc(-c4ccccc4)nc(-c4cccc(-n5c6ccccc6c6ccc7c8ccccc8sc7c65)c4)n3)c2)cc1]

The procedure of the synthesis of Comparative Compound 4 was repeated except for using 12-([1,1′-biphenyl]-3-yl)-4-(3-chlorophenyl)-6-phenyl-1,3,5-triazine in place of 2-(5-chloro-[1,1′-biphenyl]-3-yl)-4,6-diphenyl-1,3,5-triazine. The obtained Comparative Compound 5 (53% yield, pale yellow solid) was identified by FD-MS (field desorption mass spectrometry), maximum ultraviolet absorption wavelength (UV(PhMe) λmax) in toluene, and maximum fluorescence wavelength (FL(PhMe, λex=322 nm) λmax) in toluene. The results are shown below.

FDMS: calcd. for C451-128N4S=656, found m/z=656 (M+)

### UV(PhMe) λmax: 365 nm

FL(PhMe, λex=322 nm) λmax: 450 nm

1. A polycyclic compound represented by formula (Ia) or (Ib): 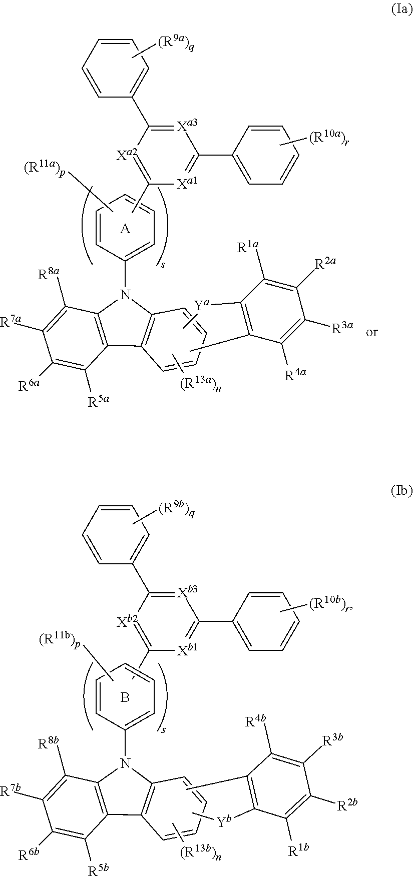
wherein
R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a}, and R^{13b} each independently represents hydrogen, a substituted or unsubstituted alkyl group having 1 to 25 carbon atoms, a substituted or un substituted carbocyclic group having 5 to 18 ring carbon atoms, a substituted or unsubstituted heterocyclic group having 5 to 18 ring atoms, or CN;
R^{9a}, R^{10a}, R^{9b} and R^{11b} each independently represents hydrogen, a substituted or unsubstituted alkyl group having 1 to 25 carbon atoms, a substituted or unsubstituted carbocyclic group having 5 to 18 ring carbon atoms, a substituted or unsubstituted heterocyclic group having 5 to 18 ring atoms, or CN, or
two adjacent groups R^{9a} and/or two adjacent groups R^{10a}, or two adjacent groups R^{9b} and/or two adjacent groups R^{10b} can form together a substituted or unsubstituted carbocyclic or heterocyclic ring;
R^{11a} and R^{11b} each independently represents hydrogen, a substituted or unsubstituted alkyl group having 1 to 25 carbon atoms, a substituted or unsubstituted carbocyclic group having 5 to 18 ring carbon atoms, or CN,
or
two groups R^{11a} on adjacent rings A or two groups R^{11b} on adjacent rings B can form together a carbocyclic ring represented by any one of the following formulae: 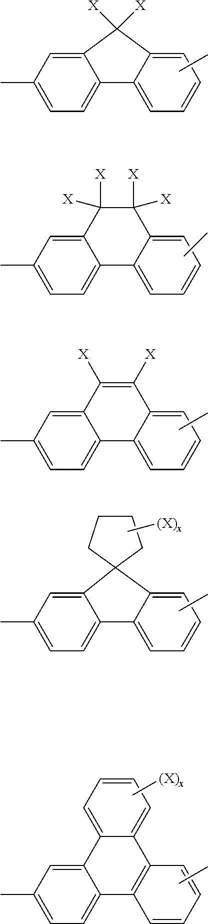
wherein each X represents hydrogen or a heteroatom free substituent and x is 0 to 8, provided that two adjacent groups X do not form together a ring, and/or
two adjacent groups R^{11a} on one of the rings A or two adjacent groups R^{11b} on one of the rings B can form together a substituted or unsubstituted carbocyclic ring;
X^{a1}, X^{a2}, X^{a3}, X^{b1}, X^{b2} and X^{b3} each represents N;
Y^{a} and Y^{b} each independently represents S or O;
n is 1 or 2;
q and r are each independently 1, 2, 3, 4 or 5;
p is 1, 2, 3 or 4; and
s is 2.

2. The compound according to claim 1, wherein R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted carbocyclic group having 5 to 18 ring carbon atoms, a substituted or unsubstituted heterocyclic group having 5 to 18 ring atoms, or CN.

3. The compound according to claim 1, wherein R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a}, and R^{13b} each independently represents hydrogen, a substituted or unsubstituted phenyl group, CN, a substituted or unsubstituted biphenyl group, a substituted or unsubstituted terphenyl group, a substituted or unsubstituted triphenylenyl group, a substituted or unsubstituted naphthyl group, a substituted or unsubstituted phenanthrenyl group, a substituted or unsubstituted dibenzofuranyl group, or a substituted or unsubstituted dibenzothiophenyl group.

4. The compound according to claim 1, wherein R^{1a}, R^{2a}, R^{3a}, R^{4a}, R^{5a}, R^{6a}, R^{7a}, R^{8a}, R^{1b}, R^{2b}, R^{3b}, R^{4b}, R^{5b}, R^{6b}, R^{7b}, R^{8b}, R^{13a} and R^{13b} each independently represents hydrogen, a substituted or unsubstituted phenyl group, a substituted or unsubstituted biphenyl group, or CN.

5. The compound according to claim 1, wherein R^{9a}, R^{10a}, R^{9b} and R^{10b} each independently represents hydrogen, a substituted or unsubstituted carbocyclic group having 5 to 18 ring carbon atoms, a substituted or unsubstituted heterocyclic group having 5 to 18 ring atoms, or CN, or
two adjacent groups R^{9a}, and/or two adjacent groups R^{10a}, or two adjacent groups R^{9b}, and/or two adjacent groups R^{10b} can form together a substituted or unsubstituted carbocyclic or heterocyclic ring.

6. The compound according to claim 1, wherein R^{9a}, R^{10a}, R^{9b} and R^{10b} each independently represents hydrogen, a substituted or unsubstituted phenyl group, a substituted or unsubstituted biphenyl group, a substituted or unsubstituted terphenyl group, a substituted or unsubstituted triphenylenyl group, a substituted or unsubstituted naphthyl group, a substituted or unsubstituted phenanthrenyl group, a substituted or unsubstituted dibenzofuranyl group, or a substituted or unsubstituted dibenzothiophenyl group, or
two adjacent groups R^{9a} and/or two adjacent groups R^{10a}, or two adjacent groups R^{9b} and/or two adjacent groups R^{10b} can form, together with the phenyl group to which R^{9a}, R^{10a}, R^{9b} and R^{10b} are attached, a substituted or unsubstituted dibenzofuranyl group, a substituted or unsubstituted dibenzothiophenyl group, or a substituted or unsubstituted fluorenyl group.

7. The compound according to claim 5, wherein R^{9a}, R^{10a}, R^{9b} and R^{10b} each independently represents hydrogen, an unsubstituted phenyl group, an unsubstituted biphenyl group, an unsubstituted dibenzofuranyl group, or an unsubstituted dibenzothiophenyl group, or
two adjacent groups R^{9a} and/or two adjacent groups R^{10a}, or two adjacent groups R^{9b} and/or two adjacent groups R^{10b} can form, together with the phenyl group to which R^{9a}, R^{10a}, R^{9b} and R^{10b} are attached, a substituted or unsubstituted dibenzofuranyl group, a substituted or unsubstituted dibenzothiophenyl group, or a substituted or unsubstituted fluorenyl group.

8. The compound according to claim 1, wherein R^{11a} and R^{11b} each independently represents hydrogen, a substituted or unsubstituted carbocyclic group having 5 to 18 ring carbon atoms, or CN,
or
two groups R^{11a} on adjacent rings A or two groups R^{11b} on adjacent rings B can form together the carbocyclic ring, or
two adjacent groups R^{11a} on one of the rings A or two adjacent groups R^{11b} on one of the rings B can form together a substituted or unsubstituted carbocyclic ring.

9. The compound according to claim 1, wherein R^{11a} and R^{11b} each independently represents hydrogen, an unsubstituted phenyl group, or an unsubstituted biphenyl group, or
two groups R^{11a} on adjacent rings A or two groups R^{11b} on adjacent rings B can form together the carbocyclic ring, or
two adjacent groups R^{11a} on one of the rings A or two adjacent groups R^{11b} on one of the rings B can form together a substituted or unsubstituted phenyl ring.

10. The compound according to claim 1, wherein R^{11a} and R^{11b} each independently represents hydrogen or an unsubstituted phenyl group, or
two groups R^{11a} on adjacent rings A or two groups R^{11b} on adjacent rings B can form together the carbocyclic ring, or
two adjacent groups R^{11a} on one of the rings A or two adjacent groups R^{11b} on one of the rings B can form together a substituted or unsubstituted phenyl ring.

11. The compound according to claim 3, wherein R^{11a} and R^{11b} each independently represents hydrogen or an unsubstituted phenyl group, or
two groups R^{11a} on adjacent rings A or two groups R^{11b} on adjacent rings B can form together the carbocyclic ring, or
two adjacent groups R^{11a} on one of the rings A or two adjacent groups R^{11b} on one of the rings B can form together an unsubstituted phenyl ring.

12. A material for an organic electroluminescence device, comprising at least one compound according to claim 4.

13. An organic electroluminescence device comprising at least one compound according to claim 1.

14. The organic electroluminescence device according to claim 13, comprising a cathode, an anode and one or more organic thin film layers comprising an emitting layer disposed between the cathode and the anode, wherein at least one layer of the one or more organic thin film layers comprises the at least one compound.

15. The organic electroluminescence device according to claim 14, wherein the one or more organic thin film layers comprise an electron-transporting zone provided between the emitting layer and the cathode, wherein the electron-transporting zone comprises the at least one compound.

16. The organic electroluminescence device according to claim 15, wherein the electron-transporting zone comprises an electron-transporting layer provided between the emitting layer and the cathode, wherein the electron-transporting layer comprises the at least one compound.

17. The organic electroluminescence device according to claim 15, wherein the electron-transporting zone comprises an electron-transporting layer and a hole-blocking layer provided between the emitting layer and the electron-transporting layer, wherein the hole-blocking layer comprises the at least one compound.

18. The organic electroluminescence device according to claim 15, wherein the electron-transporting zone further comprises at least one of an electron-donating dopant and an organic metal complex, wherein the at least one of an electron-donating dopant and an organic metal complex is at least one selected from the group consisting of an alkali metal, an alkali metal compound, an alkali metal complex, an alkaline earth metal, an alkaline earth metal compound, an alkaline earth metal complex, a rare earth metal, a rare earth metal compound, and a rare earth metal complex.

19. An electronic equipment comprising the organic electroluminescence device according to claim 13.

20. (canceled)
